(19) United States
(12) Patent Application Publication
(10) Pub. No.: US 2015/0290233 A1
(43) Pub. Date: Oct. 15, 2015

(54) METHODS OF PREVENTING TUMOR METASTASIS, TREATING AND PROGNOSING CANCER AND IDENTIFYING AGENTS WHICH ARE PUTATIVE METASTASIS INHIBITORS

(71) Applicant: Yeda Research and Development Co. Ltd., Rehovot (IL)

(72) Inventors: Yosef Yarden, Rehovot (IL); Nir Ben-Chetrit, Rehovot (IL)

(73) Assignee: Yeda Research and Development Co.Ltd. at the Weizmann Institute of Science, Rehovot (IL)

(21) Appl. No.: 14/432,520

(22) PCT Filed: Nov. 28, 2013
(86) PCT No.: PCT/IL2013/050986

§ 371 Date: Mar. 31, 2015

Related U.S. Application Data
(60) Provisional application No. 61/731,003, filed on Nov. 29, 2012.

(51) Int. Cl. A61K 31/7048; A61K 31/353; A61K 31/7034; A61K 31/704; A61K 31/12; G01N 33/574; A61K 31/155; A61K 31/4709; A61K 31/235; A61K 31/05; A61K 39/00; A61K 45/06; A61K 31/7032

(52) U.S. Cl. CPC A61K 31/7048; A61K 45/06; A61K 31/353; A61K 31/7034; A61K 31/704; A61K 31/12; A61K 31/7032; A61K 31/155; A61K 31/4709; A61K 31/235; A61K 31/05; A61K 39/0011; G01N 33/57415

(57) ABSTRACT

A method of preventing tumor metastasis with the proviso that the tumor is not glioma is provided. The method comprising administering to a subject in need thereof a therapeutically effective amount of an inhibitor of synaptojanin 2 (SYNJ2), thereby preventing tumor metastasis. Also, provided is a method of treating cancer. The method comprising, administering to a subject in need thereof a therapeutically effective amount of an inhibitor of synaptojanin 2 (SYNJ2) and an inhibitor of a cell surface receptor associated with an onset or progression of cancer, thereby treating cancer.

### FIELD AND BACKGROUND OF THE INVENTION

The present invention, in some embodiments thereof, relates to methods of preventing tumor metastasis, treating and prognosing cancer and identifying agents which are putative metastasis inhibitors.

Cell motility supports a variety of physiological and pathological processes, including tumor metastasis (Ridley, 2011). The onset of migration is driven by actin polymerization and Rho-family GTPases, which instigate formation of lamellipodia and filopodia. Burgeoning evidence implicates another type of actin-driven protrusions, called invadopodia, in matrix degradation (Murphy and Courtneidge, 2011). To seed metastases, migratory breast cancer cells form invadopodia and infiltrate into nearby vessels. Studies aiming to characterize gene expression signatures associated with metastasis of breast cancer cells to lungs (Minn et al., 2005) and brain (Bos et al., 2009) identified sets of genes that underlie site-specific metastasis. Interestingly, both sets include members of the epidermal growth factor (EGF) family, suggesting that signaling by the shared receptor, EGFR, supports metastatic dissemination.

Intracellular trafficking emerges as a key feature of cell migration and tumor progression (Mosesson et al., 2008). For example, it has been shown that mutant p53 promotes metastasis through enhanced integrin and EGFR trafficking, which depend on the Rab-coupling protein (RCP) (Muller et al., 2010). Along with Rab proteins, phosphoinositides play pivotal roles in cellular compartmentalization by determining vesicles identity (Yuan and Cantley, 2008). For example, phosphorylation at the D3 position of PI(4,5)P₂ (phosphatidyl-inositol 4,5-bisphosphate) by phosphatidylinositol 3-kinase (PI3K) generates PI(3,4,5)P₃, which is necessary for invadopodia formation (Yamaguchi et al., 2011). Similarly, PI(4,5)P₂ regulates multiple proteins controlling endocytosis and actin dynamics (Saarikangas et al., 2010), but its levels are stringently controlled by two additional types of enzymes: phospholipase C (PLCγ) promotes PI(4,5)P₂ hydrolysis, which activates Cofilin (an actin-severing protein) and drives mammary cell migration (van Rheenen et al., 2007). Likewise, inositol polyphosphate 5-phosphatases, such as Synaptojanin 2 (SYNJ2), dephosphorylate the D5 position of the inositol ring and control glioma cell migration (Chuang et al., 2004; Malecz et al., 2000). In addition homozygous mutations were identified in certain prostate cancer samples Rossi et al. Cancer Genet Cytogenet. 2005 September; 161(2):97-103.

### SUMMARY OF THE INVENTION

According to an aspect of some embodiments of the present invention there is provided a method of preventing tumor metastasis with the proviso that the tumor is not glioma, the method comprising administering to a subject in need thereof a therapeutically effective amount of an inhibitor of synaptojanin 2 (SYNJ2), thereby preventing tumor metastasis.

According to an aspect of some embodiments of the present invention there is provided a method of treating cancer the method comprising, administering to a subject in need thereof a therapeutically effective amount of an inhibitor of synaptojanin 2 (SYNJ2) and an inhibitor of a cell surface receptor associated with an onset or progression of cancer, thereby treating cancer.

According to an aspect of some embodiments of the present invention there is provided an inhibitor of synaptojanin 2 (SYNJ2) for preventing tumor metastasis with the proviso that said tumor is not glioma.

According to an aspect of some embodiments of the present invention there is provided an inhibitor of synaptojanin 2 (SYNJ2) and an inhibitor of a cell surface receptor associated with an onset or progression of cancer for treating cancer.

According to some embodiments of the invention, the cell surface receptor associated with the onset or progression of cancer is a receptor tyrosine kinase.

According to some embodiments of the invention, the receptor tyrosine kinase is an ErbB receptor.

According to some embodiments of the invention, the ErbB receptor is Epidermal Growth Factor Receptor (EGFR).

According to an aspect of some embodiments of the present invention there is provided a method of identifying a putative inhibitor of tumor metastasis, the method comprising analyzing SYNJ2-mediated processing of PI(3,4,5)P₃ to PI(3,4)P₂ in the presence of a test agent, wherein a decreased processing of PI(3,4,5)P₃ to PI(3,4)P₂ in the presence of the test agent as compared to same in an absence thereof is indicative of a putative inhibitor of tumor metastasis.

According to some embodiments of the invention, the analyzing SYNJ2-mediated processing of PI(3,4,5)P₃ to PI(3,4)P₂ is performed by a competition assay.

According to some embodiments of the invention, the competition assay assays displacement of a PI(3,4)P₂ binding domain from a complex comprising the PI(3,4)P₂ binding domain bound to PI(3,4)P₂.

According to some embodiments of the invention, the competition assay is a fluorescence polarization competitive assay.

According to an aspect of some embodiments of the present invention there is provided a method of prognosing cancer in a subject in need thereof, the method comprising determining a level or activity of SYNJ2 in a cancer cell of the subject, wherein an upregulation in the level of activity of the SYNJ2 in the cancer cell of the subject compared to same in a cell of an unaffected control sample, is indicative of a poor prognosis.

According to some embodiments of the invention, the method further comprises augmenting the prognosis using a Gold standard method.

According to some embodiments of the invention, the Gold standard method comprises detection of a marker.

According to some embodiments of the invention, the marker is selected from the group consisting of HER-2 and estrogen receptor (ER).

According to some embodiments of the invention, the metastasis is EGF dependent.

According to some embodiments of the invention, the cancer is breast cancer.

According to some embodiments of the invention, the inhibitor of SYNJ2 is selected from the group consisting of a small molecule, an antibody, a peptide and a nucleic acid silencing agent.

According to some embodiments of the invention, the small molecule is selected from the molecules listed in Table 2.

According to an aspect of some embodiments of the present invention there is provided an article of manufacture for the treatment of cancer or prevention of cancer metastasis, comprising a packaging material packaging an inhibitor of SYNJ2 and an inhibitor of a cell surface receptor associated with an onset or progression of cancer.

According to some embodiments of the invention, the inhibitor of the cell surface receptor associated with the onset or progression of cancer is an antibody.

According to some embodiments of the invention, the inhibitor of the cell surface receptor associated with the onset or progression of cancer is a small molecule inhibitor.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-I show that EGF promotes invasive growth of mammary cells and induces a specific set of genes. FIG. 1A—MCF10A cells were plated in the absence of growth factors and allowed to form clusters. Seventy-two hours later, cells were treated with the indicated growth factors (each at 10 ng/mL) and phase contrast images were taken 24 hours later (scale bar, 50 μm). FIG. 1B—MCF10A cells were plated in migration or invasion chambers, as indicated, in the presence of the indicated ligands (10 ng/mL), and 18 hours later cells that migrated to the lower compartment were stained with crystal violet (left panel). Shown are quantifications of migration and invasion signals, normalized to the effect of EGF treatment. Data represent mean±S.D. of biological triplicates from a representative experiment that was repeated twice (right panel). FIG. 1C—MCF10A cells were plated in transwell inserts in EGF-containing medium, without or with the inhibitors AG-1478 (1 μM), U0126 (5 μM), or Wortmannin (200 nM), and allowed to migrate for 18 hours. Data represent mean±S.D. of triplicates. The experiment was repeated twice. FIG. 1D—A list of 425 genes specifically induced in human mammary MCF10A cells by EGF (and not by serum (Amit et al., 2007)), was intersected with genes that were up-regulated in the context of metastasis of MDA-MB-231 cells (1,597 genes) (Minn et al., 2005). One of the 23 overlapping genes encodes the 5′-phosphatidylinositol lipid phosphatase Synaptojanin-2 (SYNJ2). FIG. 1E—MCF10A cells were infected with lentiviral particles encoding LacZ (Ctrl) or SYNJ2-GFP (SYNJ2-OX). Expression levels of the endogenous SYNJ2 and the SYNJ2-GFP fusion protein were determined by immunoblotting, and equal protein loading was confirmed by probing for tubulin. FIG. 1F—The Ctrl and SYNJ2-OX clones of MCF10A cells were plated in migration chambers (5×10⁴ cells/well) in the absence (NT) or presence of EGF (10 ng/mL) and allowed to migrate for 22 hours. Migrating cells that reached the other side of the filter were stained with crystal violet and images were taken. FIG. 1G—MCF10A cells were transfected with siRNA control (siCtrl) or siRNA directed to SYNJ2 (siSYNJ2), and protein levels of SYNJ2 were determined 36 hours later by immunoblotting. Equal protein loading was confirmed by immunoblotting for Ras-GAP. FIG. 1H—The cells presented in G were plated in migration chambers (5×10⁴ cells/well) in the absence (NT) or presence of EGF (10 ng/mL) and allowed to migrate for 22 hours. Migrating cells that reached the lower face of the filter were stained with crystal violet and images were captured. FIG. 1I—Confluent cultures of MCF10A cells were treated with the indicated siRNAs. Once monolayers formed, they were subjected to an automated scratching system that monitors the rate of scratch closure.

FIGS. 2A-E show that transcriptional induction of SYNJ2 by EGF promotes invasive growth. FIG. 2A—Serum-starved MCF10A cells were stimulated with EGF (20 ng/mL) or serum (5%), and SYNJ2 mRNA expression was assayed by using microarrays or RT-qPCR. FIG. 2B—MCF10A cells were stimulated with EGF, extracted and immunoblotted as indicated. FIG. 2C—MCF10A cells, infected with viruses encoding GFP-SYNJ2 (SYNJ2-OX) or LacZ as control (Ctrl), were cultured for 4 days in the absence or presence of EGF. Phase contrast (top, bar: 100 μm) and confocal images (bottom, bar: 20 μm) using phalloidin and DAPI were obtained. FIGS. 2D-E—MCF10A cells were cultured for 22 hours in migration or invasion chambers (5-6×10⁴ cells/well) in the absence (NT) or presence of EGF (10 ng/mL). Cells that reached the filter's bottom were stained and filter's coverage quantified (mean±S.D.).

FIGS. 3A-G show inducible translocation of SYNJ2 to the leading edge accompanies mammary cell migration and invasion. FIG. 3A—MDA-MB-231 cells were infected with lentiviral particles encoding LacZ (Ctrl) or a V5-tagged SYNJ2 (SYNJ2-V5), along with control shRNA (shCtrl) or an shRNA directed against SYNJ2 (shSYNJ2). Protein levels of V5-SYNJ2 and endogenous SYNJ2 were determined by immunoblotting. Equal protein loading was confirmed by immunoblotting for AKT. FIG. 3B—Phase images (left panels) and invasion images (right panels) of MDA-MB-231 cells stably over-expressing SYNJ2, or LacZ as control. The invasive capacities were determined in triplicates using an invasion assay, and invading cells were quantified and normalized to control (Ctrl). Scale bar, 50 μm. FIG. 3C—MDA-MB-231 cells were transfected with siRNA oligonucleotides directed to SYNJ2 (or siCtrl). Following 36 hours, protein levels of SYNJ2 were determined by immunoblotting. Equal protein loading was confirmed by immunoblotting for Ras-GAP. FIG. 3D—Cells from C were plated in migration or invasion chambers and incubated for 18 hours. The migration and invasion signals were quantified and normalized to EGF-treated siCtrl cells. Data shown are means±S.D. of triplicates. FIG. 3E—MDA-MB-231 cells transiently expressing GFP-SYNJ2 were plated on glass coverslips and stimulated with TGFα (10 ng/mL). Time-lapse microscopy photos were taken (every 10 seconds). The images shown are inverted, with black spots representing SYNJ2 and its assembly at the base of lamellipodia. Scale bar, 10 μm. FIG. 3F—MDA-MB-231 cells were immunostained for endogenous SYNJ2 and F-actin using TRITC-phalloidin. The squared area is magnified. Scale bar, 10 μm. FIG. 3G—MCF10A cells were stimulated with EGF for 18 hours, and then immunostained for endogenous SYNJ2 and counter-stained for F-actin using TRITC-phalloidin. Scale bar, 10 μm.

FIGS. 4A-F show that the catalytic activity of SYNJ2 is essential for invasive growth. FIGS. 4A-B—MDA-MB-231 cells expressing SYNJ2 (SYNJ2-OX) or shRNA to SYNJ2 (shSYNJ2), as well as control cells, were seeded in 5% Matrigel. Images were captured after six days, and invasive spheroids quantified (mean±S.D.). Scale bars, 50 μm. FIGS. 4C-D—shSYNJ2-expressing MDA-MB-231 cells were infected with WT SYNJ2 (shSYNJ2+SYNJ2^{WT}) or with a catalytically disabled mutant (shSYNJ2+SYNJ2^{CD}). Cells were either extracted and immunoblotted as indicated, or they were allowed to invade for 18 hours in invasion chambers. Images of the invaded cells and their normalized quantification are shown (mean±S.D). FIG. 4E—show scanning electron micrographs of shCtrl and shSYNJ2 cells grown on fibronectin. Scale bar, 2 μm. FIG. 4F—Images of F-actin in the indicated MDA-MB-231 cells stained with phalloidin and DAPI. Z-axis sections (lines) and magnified areas are shown. Arrowheads mark swollen structures. Scale bar, 10 μm.

FIGS. 5A-H show the subcellular localization of SYNJ2. FIG. 5A—MDA-MB-231 cells expressing GFP-SYNJ2 were transfected with an RFP-Clathrin and plated on fibronectin-coated plates. Using spinning-disc microscopy, cells were imaged every five seconds. Arrowheads mark a newly formed leading edge. Scale bar, 5 μm. FIG. 5B—Representative time frames depicting assembly and disassembly of SYNJ2 at the leading edge (upper two rows) and underneath the cell body. For the lower rows, cells were transfected with a mCherry-lifeACT plasmid and plated on collagen. Thereafter, cells were imaged at 1 minute intervals. Arrowheads were inserted for reference. Note the difference in time scales. Scale bar, 1 μm. FIG. 5C—Cells were simultaneously imaged by TIRF and epifluorescence microscopy and signals converted into kymographs (X-axis). Arrowheads mark signal initiation. Scale bar, 5 μm. FIG. 5D—Cells were imaged using spinning disc confocal microscopy 5 minutes before and 5 minutes after treatment with Dyngo-4a (30 μM; a Dynamin-2 inhibitor). Scale bar, 5 μm. FIG. 5E—MDA-MB-231 cells stably expressing GFP-SYNJ2 were pre-incubated with Dyngo-4a (30 μM; 30 min), or with solvent (DMSO). Cell lysates were subjected to immunoprecipitation with anti-GFP antibodies (or with no antibody; -Ab), and then immunoblotted, along with a sample (5%) of the cell lysate, with the indicated antibodies. FIG. 5F—Cells were plated on fibronectin, fixed and immunostained for endogenous Rac1. Scale bar, 10 μm. FIG. 5G—Cells were imaged using confocal microscopy 5 minutes prior to and 5 minutes after a 30 min-long treatment with NSC-23766 (504). Scale bar, 5 μm. FIG. 5H—MDA-MB-231 cells were treated with the indicated siRNA oligonucleotides. Cell extracts were blotted for SYNJ2 and Ras-GAP. GTP-Rac1 levels were determined using an ELISA-based assay (Cytoskeleton).

FIGS. 6A-D show SYNJ2 localization to the leading edge is distinct from caveolins distribution and depends on F-actin, cholesterol and PI3K. FIG. 6A—MDA-MB-231 cells expressing GFP-SYNJ2 and co-expressing RFP-Cav1 were simultaneously imaged over time, and signals converted into kymographs (X and Y axis). Note the transient nature of SYNJ2 assemblies and stable appearance of Caveolin 1. Scale bar, 5 μm. FIG. 6B—The left panel depicts the distribution (% of pits versus lifetime) of 150 randomly selected SYNJ2 assemblies, imaged as in FIG. 5A (5 second intervals, single plane, spinning disk confocal). The right panel depicts the average (±SEM) relative intensity of assemblies that showed a 55 seconds lifetime. FIG. 6C—MDA-MB-231 cells stably-expressing GFP-SYNJ2 were treated with MβCD (10 mM, 15 minutes) or with Wortmannin (500 nM, 15 minutes). Images of the same selected cells were captured every 6 seconds, either prior to or following treatment, and signals were converted into kymographs (representing the squared insets in the left panels). Scale bar, 20 μm. FIG. 6D—MDA-MB-231 cells stably co-expressing GFP-SYNJ2 and lifeACT-mCherry were treated with LatrunculinB (1 μM, 15 minutes). Images were acquired either prior to or following treatment. Scale bar, 5 μm.

FIGS. 7A-E show SYNJ2 depletion arrests EGFR in intracellular vesicles FIG. 7A—MCF10A cells stably expressing shRNA control (shCtrl) or shRNA specific to SYNJ2 (shSYNJ2) were extracted three days after plating in EGF-containing medium Immunoblots were probed for SYNJ2, EGFR, phosphorylated tyrosine 1068 of EGFR (pEGFR), phosphorylated ERK (pERK), and Ras-GAP, as a loading control. FIG. 7B—MCF10A cells were transfected with siRNA control, or siRNA directed against SYNJ2, in the presence of EGF. Confocal immunofluorescence analysis was performed using EGFR and SYNJ2 antibodies. Note that only the SYNJ2-depleted cell (asterisk) displays EGFR trafficking defects. Scale bar, 10 μm. FIG. 7C—Three derivatives of MDA-MB-231 cells were immunostained for EGFR and counterstained for DAPI and F-actin: (i) cells in which SYNJ2 was knocked-down (shSYNJ2; left column), (ii) the same cells infected by lentiviral gene transfer corresponding to the catalytically-dead form (shSYNJ2+SYNJ2^{CD}; middle column), and (iii) cells in which SYNJ2 was knocked-down and the wild type form was introduced by infection (shSYNJ2+SYNJ2^{WT}; right column). Scale bar, 20 μm. FIG. 7D—Ubiquitinated EGFR levels (densitometry). FIG. 7E—MDA-MB-231 derivatives were stimulated with 488-Tfn (5 minutes, 10 μg/mL). Cells were fixed on ice, acid-washed and analysed for signal intensity.

FIGS. 8A-I show that SYNJ2 regulates EGFR trafficking and chemotaxis.

FIG. 8A—Whole extracts of MDA-MB-231 cells transfected with the indicated siRNAs were immunoblotted as indicated. FIG. 8B—FACS (left) and ¹²⁵I-EGF binding (right; in triplicates) analyses of surface EGFR in the indicated MDA-MB-231 subclones. FIG. 8C—shCtrl and shSYNJ2 cells were grown on fibronectin and immunostained for EGFR and F-actin. Bar, 20 μm. FIG. 8D—Rose plots of tracks of shCtrl and shSYNJ2 MDA-MB-231 cells, which migrated in chemotaxis chambers upon exposure to an EGF gradient. The red tracks indicate cells migrating toward EGF. FIG. 8E—Starved MDA-MB-231 derivatives were treated with EGF (10 ng/mL) and cell lysates were subjected to immunoprecipitation and immunoblotting as indicated. FIG. 8F—Cells were cultured as in C and immunostained for active EGFR (pY1045) and F-actin. Bar, 10 μm. FIG. 8G—The indicated MDA-MB-231 derivatives were treated with EGF (10 ng/ml) for 5 hours and extracts immunoblotted as indicated. FIG. 8H—The indicated MDA-MB-231 derivatives were exposed to Alexa Fluor 488-Tfn (25 μg/ml; 5 min), acid-washed to remove surface-bound ligands, and images taken at the indicated intervals. Normalized fluorescence signals are shown. Bar, 10 μm. FIG. 8I—MDA-MB-231 cells, pre-treated with siCtrl or siSYNJ2, were stimulated with Alexa Fluor 488-EGF (20 μg/ml; 10 min), acid-washed, incubated at 37° C. for the indicated intervals and analysed by FACS.

FIGS. 9A-D show that SYNJ2 is necessary for both vesicular trafficking and focal adhesion formation. FIG. 9A—MDA-MB-231 derivatives (shCtrl and shSYNJ2) were fixed and stained for EEA1, F-actin and nuclei (DAPI). Scale bar, 10 μm. FIG. 9B—MDA-MB-231 derivatives, namely shCtrl and shSYNJ2 cells, were probed for integrin beta-1, F-actin and DAPI (scale bar, 20 μm). FIG. 9C—MDA-MB-231 cells were treated with siCtrl and siSYNJ2 for 48 hours and then immunostained for integrin beta-1 and phosphorylated EGFR. FIG. 9D—Immunofluorescence analysis of MDA-MB-231 derivatives for paxillin, nuclei (DAPI), and F-actin (using TRITC-phalloidin). The paxillin signal was quantified in cytoplasmic regions relative to focal adhesions, and the numbers of focal adhesions per cell were also quantified. In addition, the shapes of focal adhesions were quantified by determining deviations from a perfect circle (eccentricity). Scale bar, 10 μm.

FIGS. 10A-F show that SYNJ2 depletion perturbs phosphoinositide homeostasis, inflates early endosomes and disassembles focal adhesions. FIG. 10A—MDA-MB-231 cells, expressing shCtrl or shSYNJ2, were transfected with a GFP-Rab4 plasmid and 48 hours later cells were fixed and counterstained for F-actin using TRITC-phalloidin. FIG. 10B—MDA-MB-231 derivatives were immunostained for Rab5, F-actin and nuclei (DAPI). Images were quantified for the size and number of Rab5-positive vesicle, as well as for the average cell area. Scale bars, 10 μm. FIG. 10C—Phosphoinositides extracted from ³H-phosphoinositol labeled derivatives of MDA-MB-231 cells, were separated by chromatography and their levels determined in three different experiments (signals normalized to shCtrl cells). FIG. 10D—shCtrl and shSYNJ2 MDA-MB-231 cells were probed for pY1068-EGFR, Paxillin and F-actin (co-localization signal are white). Scale bar, 10 μm). FIG. 10E—shCtrl and shSYNJ2 MDA-MB-231 cells were seeded. Unattached cells were removed 20 min later and attached cells were imaged and quantified for surface area. FIG. 10F—MDA-MB-231 cells, stably expressing shCtrl or shSYNJ2, were plated on RTCA E-plates and real-time impedance measurements were recorded in 5 sec intervals for 80 min, and then in 10 min intervals for additional 80 min. Means of 2 replicates (±S.D.) are shown.

FIGS. 11A-G show that SYNJ2 regulates protease secretion and invadopodium assembly. FIG. 11A—shCtrl and shSYNJ2 MDA-MB-231 cells were cultured in Matrigel for 5 days, fixed and immunostained for MMP-9. Signal intensities were converted into heat-maps and plotted against distance from colony cores. Arrowheads mark spheroid boundaries. Bar, 50 μm. FIG. 11B—Supernatants from control MDA-MB-231 cells and cells stably overexpressing SYNJ2 were analyzed in triplicates for MMP-2 and MMP-9 activity using gelatin zymography. FIG. 11C—MDA-MB-231 cells stably expressing GFP-SYNJ2 were plated onto coverslips pre-coated with cross-linked fluorescent gelatin. Three hours later, cells were probed for GFP and F-actin, and invadopodial structures detected (arrowheads). Bar, 10 μm. FIG. 11D—MDA-MB-231 cells overexpressing SYNJ2 (SYNJ2-OX), as well as cells pre-treated with siCtrl or siSYNJ2 oligonucleotides, were plated on coverslips pre-coated with cross-linked fluorescent gelatin and invadopodial structures were quantified in three independent experiments. FIG. 11E—Invadopodial structures of MDA-MB-231 cells treated with the indicated siRNAs were detected by gelatin degradation, as well as by staining for F-actin or TKS5. Arrowheads (Z-axis images) mark invadopodia. Bar, 10 μm. FIG. 11F—MDA-MB-231 cells expressing siCtrl or siSYNJ2 were plated on gelatin-coated coverslips and processed as in C using phalloidin and antibodies to the phosphorylated form of EGFR (tyrosine 1068). Scale bar, 10 μm. FIG. 11G—Media conditioned over 3 days by the indicated MDA-MB-231 derivatives were examined using an ELISA-based assay for EGF-like ligands.

FIGS. 12A-G show that SYJN2 regulates matrix degradation and invadopodia assembly. FIG. 12A—The indicated siRNA-treated MDA-MB-231 cells were plated in triplicates, cultured for 3 days and their conditioned media were separated electrophoretically using a gelatin (0.1%) embedded gel, followed by protein staining to quantify MMP-2 and MMP-9 proteolytic activity. FIG. 12B—Co-immunoprecipitation analysis using GFP-conjugated beads and cleared extracts of MDA-MB-231 cells stably expressing GFP-SYNJ2. FIG. 12C—MDA-MB-231 cells stably expressing GFP-SYNJ2 were transfected with a RFP-Cortactin plasmid and plated on collagen plates. Live-cell image analysis was performed forty-eight hours later, and representative snapshot images of both peripheral and central cell areas were captured. Scale bar 5 μm. FIG. 12D—The indicated derivatives of MDA-MB-231 cells were transfected with a plasmid encoding a Myc-tagged PH domain of Tapp1 (a PI(3,4)P₂ binder) and 48 hours later they were plated on gelatin-coated surfaces. The co-distribution of F-actin, aggregated TKS5 and PI(3,4)P₂ (Tapp1) was visualized and quantified using confocal microscopy. Scale bar, 10 μm. FIG. 12E—MDA-MB-231 cells expressing siCtrl or siSYNJ2 were plated onto FITC-gelatin coated glass coverslips and incubated for 3 hours. Cells were then fixed and immunostained for CD44, and counter stained for F-actin with TRITC-phalloidin. Cells were visualized using fluorescence microscopy, and invadopodia were detected by observing holes in the FITC-gelatin matrix. The framed areas are enlarged. Scale bar, 10 μm. FIG. 12F—An antibody to CD44 was used for FACS analysis of surface expression by shCtrl and shSYNJ2 cells. Indicated are the fractions of cells corresponding to the framed regions. FIG. 12G—MDA-MB-231 cells pre-treated with siCtrl or siSYNJ2 were plated onto FITC-gelatin coated glass coverslips and incubated for 3 hours. Cells were then fixed and immunostained for MT1-MMP, and counter stained for F-actin with TRITC-phalloidin. Scale bar, 10 μm.

FIGS. 13A-H show that the enzymatic activity of SYNJ2 propels metastatic spread of mammary tumor cells. FIG. 13A—The indicated derivatives of RFP-expressing MDA-MB-231 cells (2×10⁶/mouse) were implanted in the fat pad of female SCID mice (10-11 per group). Tumor size (mean±S.D.) was measured 2 and 6 weeks post implantation. FIGS. 13B-C—Metastases that appeared six weeks post-implantation in axillary and distant lymph nodes (FIG. 13B), or lungs (FIG. 13C), are shown. Asterisks mark p values: *<0.05, **<0.01 and ***<0.001. FIGS. 13D-F—Control (LacZ) and SYNJ2-overexpresseing (SYNJ2-OX) RFP-labelled MDA-MB-231 cells were implanted in animals as in A and tumor size (FIG. 13D), as well as metastases to lymph nodes (FIG. 13E) and lungs (FIG. 13F) were quantified 6 and 8 weeks post implantation. FIGS. 13G-H—The indicated MDA-MB-231-RFP derivatives were injected either intravenously (1.5×10⁵ per mouse; tail vein), or in the mammary fat pad (2.5×10⁶ per mouse) of 5-week old female SCID mice. Four weeks later, lungs from mice injected into the vein were examined for RFP signals (left and middle panels). Peripheral blood was collected from the fat pad-treated group four weeks later. Samples were purified on a gradient of ficol and the numbers of RFP-positive circulating tumor cells (CTC) were scored per 1×10⁶ FACS readings and normalized to tumor weight.

FIG. 14 is an in-vivo imaging of local and distant lymph node metastases. Representative images of local (ipsilateral) and distant (contralateral) lymph node metastases in mice that were inoculated with MDA-MB-231-RFP cells and analysed 6 weeks later (see FIG. 13B). Prior to imaging, mice were anaesthetized and their fur was removed for visualization and quantification of metastases in lymph nodes.

FIG. 15 is a working model depicting the integrated action of SYNJ2 in cell migration and invasion. EGFR-loaded recycling endosomes position active receptors at the ventral membrane, and this is followed by local activation of PI3K. Phosphorylation of membranal PI(4,5)P₂ by PI3K generates PI(3,4,5)P₃, which is dephosphorylated by SYNJ2 to PI(3,4)P₂. The latter recruits TKS5, which anchors Cortactin and nucleates actin polymerization. In parallel, SYNJ2 controls delivery of adhesion molecules like CD44, and proteases like MT1-MMP, to degrade the extracellular matrix (ECM) and establish new invasive structures, the invadopodia. In a similar way, EGFR delivery to the cell periphery leads to breakdown of PI(4,5)P₂ by SYNJ2 (and phospholipase C), which locally activates Dynamin and actin severing enzymes like Cofilin to dissolve cortical actin fibres and initiate actin-filled, integrin-rich protrusions called lamellipodia. The horizontal arrow marks the direction of cell migration. Color-coded segments of the plasma membrane denote specific PI phospholipids.

FIGS. 16A-C show that SYNJ2 is highly expressed in aggressive breast tumors. FIG. 16A—Immunohistochemistry and tissue microarrays were used to stratify 331 invasive breast carcinomas according to SYNJ2 abundance (high, medium and low). The relative fraction of tumors is presented according to clinical subtypes. FIG. 16B—Representative images of SYNJ2 staining demonstrating intensities and patterns (magnified in the right column) observed in a luminal case (an asterisk marks expression by endothelial cells as control), and both basal-like and HER2-overexpressing breast tumors. FIG. 16C—Kaplan-Meier curves stratified according to SYNJ2 mRNA expression in cohorts of 286 (left; GSE2034) or 99 (right; GSE19783) breast cancer patients.

FIGS. 17A-B show the principles of the fluorescence polarization assay utilized for measuring the 5′-phosphatase activity of SYNJ2. FIG. 17A is a scheme demonstrating the general principle that an unbound PI(3,4)P2 fluorescent probe gives rise to low polarization readings, while the bound PI(3,4)P2 fluorescent probe increases the polarization readings. FIG. 17B is a representative bar graph showing SYNJ2 5′-phosphatase activity detection as measured by polarization values (mP).

FIG. 18 depicts the amino acid and nucleic acid sequences (SEQ ID NO: 13 and 14, respectively) of the Flag-TAPP1 PH domain-His that was cloned into pET28 plasmid and expressed in E. coli. The TAPP1-PH domain is marked in yellow.

### DESCRIPTION OF SPECIFIC EMBODIMENTS OF THE INVENTION

The present invention, in some embodiments thereof, relates to methods of preventing tumor metastasis, treating and prognosing cancer and identifying agents which are putative metastasis inhibitors.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Growth factors propel cell migration and metastasis, but underlying mechanisms are incompletely understood.

The present inventors have now identified synaptojanin-2 (SYNJ2) as a master module in regulating invadopodia and lamellipodia in vitro and cancer metastasis in vivo.

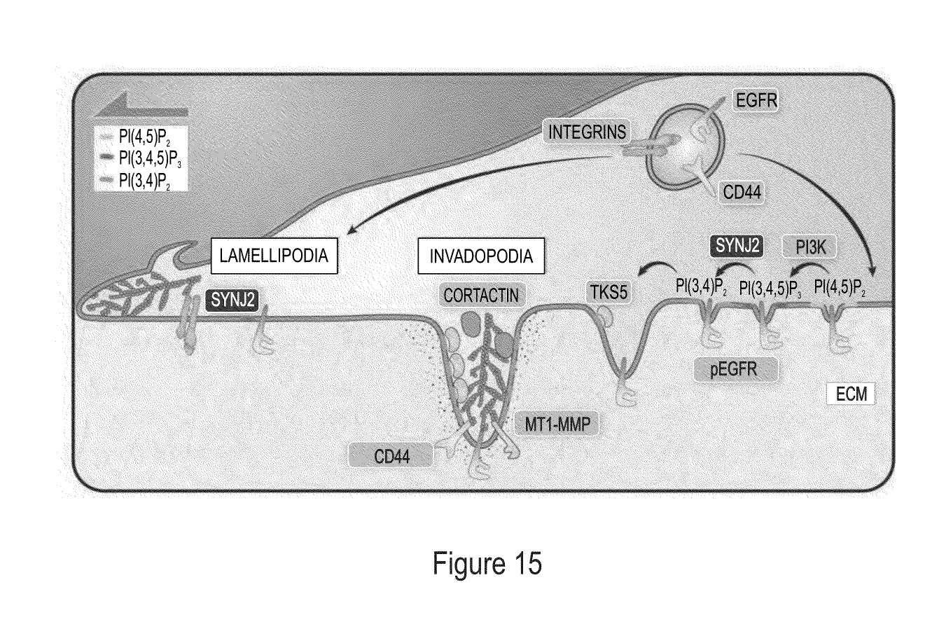

As is illustrated hereinbelow and in the Examples section which follows, the present inventors substantiated their finding in vitro, in animals and in patient specimens. Specifically, employing EGF-stimulated mammary cells the present inventors link the lipid phosphatase synaptojanin 2 (SYNJ2) to an invasive phenotype, and relate high SYNJ2, to short survival rates of cancer patients. Knockdown of SYNJ2 robustly impaired metastasis of mammary tumor cells in an animal model. In vitro, SYNJ2-depleted cells exhibited derailed trafficking of EGFR and integrins, resulting in deformed focal adhesions, arrested lamellipodia and disappearance of invadopodia. Without being bound to theory it is suggested that recycling of active EGFRs focally promotes SYNJ2-mediated dephosphorylation of specific phosphoinositol lipids, thereby instigating formation of both invadopodia and lamellipodia and facilitates tumor progression (see FIG. 15).

Thus, according to an aspect of the invention there is provided a method of preventing tumor metastasis with the proviso that said tumor is not glioma, the method comprising administering to a subject in need thereof a therapeutically effective amount of an inhibitor of synaptojanin 2 (SYNJ2), thereby preventing tumor metastasis.

As used herein the phrase “tumor metastasis” refers to a malignant tumor which spreads out of its primary location to other parts of the body e.g., breast cancer which metastasizes to the lungs.

As used herein the terms “cancer” and “tumor” are interchangeably used. The term refers to a malignant growth or tumor caused by abnormal and uncontrolled cell division.

As used herein the term “preventing” refers to arresting, halting, inhibiting the metastatic process or progression and subsequent metastasis.

According to yet another aspect, there is provided a method of treating cancer the method comprising, administering to a subject in need thereof a therapeutically effective amount of an inhibitor of synaptojanin 2 (SYNJ2) and an inhibitor of a cell surface receptor associated with an onset or progression of cancer, thereby treating cancer.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

Non-limiting examples of cancers which can be treated (or prognosed) according to some embodiments of the invention include any solid or non-solid cancer and/or cancer metastasis, including, but is not limiting to, tumors of the gastrointestinal tract (colon carcinoma, rectal carcinoma, colorectal carcinoma, colorectal cancer, colorectal adenoma, hereditary nonpolyposis type 1, hereditary nonpolyposis type 2, hereditary nonpolyposis type 3, hereditary nonpolyposis type 6; colorectal cancer, hereditary nonpolyposis type 7, small and/or large bowel carcinoma, esophageal carcinoma, tylosis with esophageal cancer, stomach carcinoma, pancreatic carcinoma, pancreatic endocrine tumors), endometrial carcinoma, dermatofibrosarcoma protuberans, gallbladder carcinoma, Biliary tract tumors, prostate cancer, prostate adenocarcinoma, renal cancer (e.g., Wilms' tumor type 2 or type 1), liver cancer (e.g., hepatoblastoma, hepatocellular carcinoma, hepatocellular cancer), bladder cancer, embryonal rhabdomyosarcoma, germ cell tumor, trophoblastic tumor, testicular germ cells tumor, immature teratoma of ovary, uterine, epithelial ovarian, sacrococcygeal tumor, choriocarcinoma, placental site trophoblastic tumor, epithelial adult tumor, ovarian carcinoma, serous ovarian cancer, ovarian sex cord tumors, cervical carcinoma, uterine cervix carcinoma, small-cell and non-small cell lung carcinoma, nasopharyngeal, breast carcinoma (e.g., ductal breast cancer, invasive intraductal breast cancer, sporadic; breast cancer, susceptibility to breast cancer, type 4 breast cancer, breast cancer-1, breast cancer-3; breast-ovarian cancer), squamous cell carcinoma (e.g., in head and neck), neurogenic tumor, astrocytoma, ganglioblastoma, neuroblastoma, lymphomas (e.g., Hodgkin's disease, non-Hodgkin's lymphoma, B cell, Burkitt, cutaneous T cell, histiocytic, lymphoblastic, T cell, thymic), gliomas, adenocarcinoma, adrenal tumor, hereditary adrenocortical carcinoma, brain malignancy (tumor), various other carcinomas (e.g., bronchogenic large cell, ductal, Ehrlich-Lettre ascites, epidermoid, large cell, Lewis lung, medullary, mucoepidermoid, oat cell, small cell, spindle cell, spinocellular, transitional cell, undifferentiated, carcinosarcoma, choriocarcinoma, cystadenocarcinoma), ependimoblastoma, epithelioma, erythroleukemia (e.g., Friend, lymphoblast), fibrosarcoma, giant cell tumor, glial tumor, glioblastoma (e.g., multiforme, astrocytoma), glioma hepatoma, heterohybridoma, heteromyeloma, histiocytoma, hybridoma (e.g., B cell), hypernephroma, insulinoma, islet tumor, keratoma, leiomyoblastoma, leiomyosarcoma, leukemia (e.g., acute lymphatic, acute lymphoblastic, acute lymphoblastic pre-B cell, acute lymphoblastic T cell leukemia, acute-megakaryoblastic, monocytic, acute myelogenous, acute myeloid, acute myeloid with eosinophilia, B cell, basophilic, chronic myeloid, chronic, B cell, eosinophilic, Friend, granulocytic or myelocytic, hairy cell, lymphocytic, megakaryoblastic, monocytic, monocytic-macrophage, myeloblastic, myeloid, myelomonocytic, plasma cell, pre-B cell, promyelocytic, subacute, T cell, lymphoid neoplasm, predisposition to myeloid malignancy, acute nonlymphocytic leukemia), lymphosarcoma, melanoma, mammary tumor, mastocytoma, medulloblastoma, mesothelioma, metastatic tumor, monocyte tumor, multiple myeloma, myelodysplastic syndrome, myeloma, nephroblastoma, nervous tissue glial tumor, nervous tissue neuronal tumor, neurinoma, neuroblastoma, oligodendroglioma, osteochondroma, osteomyeloma, osteosarcoma (e.g., Ewing's), papilloma, transitional cell, pheochromocytoma, pituitary tumor (invasive), plasmacytoma, retinoblastoma, rhabdomyosarcoma, sarcoma (e.g., Ewing's, histiocytic cell, Jensen, osteogenic, reticulum cell), schwannoma, subcutaneous tumor, teratocarcinoma (e.g., pluripotent), teratoma, testicular tumor, thymoma and trichoepithelioma, gastric cancer, fibrosarcoma, glioblastoma multiforme; multiple glomus tumors, Li-Fraumeni syndrome, liposarcoma, lynch cancer family syndrome II, male germ cell tumor, mast cell leukemia, medullary thyroid, multiple meningioma, endocrine neoplasia myxosarcoma, paraganglioma, familial nonchromaffin, pilomatricoma, papillary, familial and sporadic, rhabdoid predisposition syndrome, familial, rhabdoid tumors, soft tissue sarcoma, and Turcot syndrome with glioblastoma.

According to a specific embodiment, the cancer is breast cancer.

According to a specific embodiment, the cancer (or the cancer metastasis) is EGF-regulated.

According to another preferred embodiment, the cancer is characterized by over-expression or up-regulation of an ErbB receptor molecule such as EGFR or HER2.

Mutations that lead to EGFR overexpression (known as upregulation) or overactivity have been associated with a number of cancers, including lung cancer, anal cancers and glioblastoma multiforme. In this latter case a more or less specific mutation of EGFR, called EGFRvIII is often observed. Mutations, amplifications or misregulations of EGFR or family members are implicated in about 30% of all epithelial cancers.

Mutations involving EGFR could lead to its constant activation, which could result in uncontrolled cell division—a predisposition for cancer. Consequently, mutations of EGFR have been identified in several types of cancer, and it is the target of an expanding class of anticancer therapies [Mang 2007 J. Clin. Invest. 117 (8): 2051-8].

Amplification or over-expression of the ERBB2 gene occurs in approximately 30% of breast cancers. It is strongly associated with increased disease recurrence and a worse prognosis. Over-expression is also known to occur in ovarian, stomach, and aggressive forms of uterine cancer, such as uterine serous endometrial carcinoma.

Following is a list of cancers in which members of the ErbB family of receptor tyrosine kinases are implicated.

ErbB-1—adrenocortical cancer, biliary cancer, cervical cancer, colorectal cancer, esophageal cancer, gallbladder cancer, gastric cancer, glioblastoma, head and neck cancer, lung cancer (non-small cell, squamous cell carcinoma, adenocarcinoma, and large cell lung cancer), pancreatic cancer, salivary gland cancer, diarrhea benign neoplasm, invasive carcinoma, skin disease, ductal carcinoma in situ, paronychia.

ErbB-2—biliary cancer, bladder cancer, breast cancer, cholangiocarcinoma, esophageal cancer, gallbladder cancer, gastric cancer, glioblastoma, ovarian cancer, pancreatic cancer, salivary gland cancer. According to a specific embodiment the cancer is breast or gastric cancer.

ErbB-3—breast cancer, lung cancer and viral leukemia.

ErbB-4—breast cancer, viral leukemia, medulloblastoma, lung cancer and mammary tumor.

As used herein the term “subject” refers to a mammal (e.g., human) who has been diagnosed with cancer.

As used herein synaptojanin-2 or SYNJ2 refers to Synaptic inositol-1,4,5-trisphosphate 5-phosphatase 2, EC 3.1.3.36. Synaptojanin-2 is a ubiquitously expressed inositol polyphosphate 5-phosphatase (SEQ ID NO: 1 and 2, referring to polynucleotide and encoded polypeptide, respectively).

As used herein the phrase “inhibitor of synaptojanin 2 (SYNJ2)” refers to a molecule which decreases or downregulates the expression or activity of SYNJ2.

Downregulating can be by more than 10%, 20%, 30%, 40%, 50%, 60%, 70%, 80%, 90% or a complete inhibition (100% loss of activity or expression as determined by a given assay such as described hereinbelow).

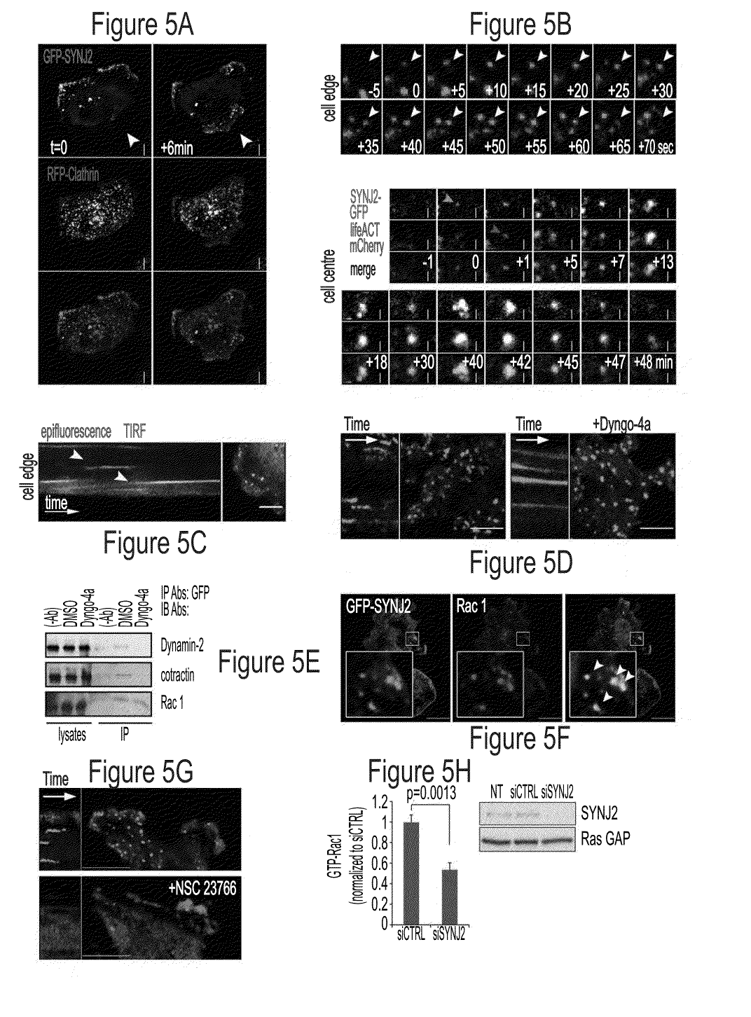

Downregulating the expression of SYNJ2 can be effected at the DNA, RNA or protein level as described hereinbelow. A SYNJ2 activity refers to its catalytic activity [as a phosphatase, converting PI(3,4,5)P₃ into PI(3,4)P₂], its signaling activity (interacts with dynamin, cotractin, see FIGS. 5E-H) or cellular localization. In the latter case the inhibitor of SYNJ2 will alter the protein's cellular localization.

Thus, downregulation of SYNJ2 can be effected on the genomic and/or the transcript level using a variety of molecules which knock-in the gene or interfere with its transcription and/or translation [e.g., nucleic acid silencing agents e.g., nucleic acid (RNA) silencing agents e.g., antisense, siRNA, shRNA, micro-RNA, Ribozyme and DNAzyme], or on the protein level using e.g., antagonists, enzymes that cleave the polypeptide and the like.

Following is a list of agents capable of downregulating expression level and/or activity of SYNJ2.

One example, of an agent capable of downregulating SYNJ2 is an antibody or antibody fragment capable of specifically binding SYNJ2. Preferably, the antibody specifically binds at least one epitope of SYNJ2. As SYNJ2 is a cellular protein, measures are taken to introduce the antibody into the cells. As used herein, the term “epitope” refers to any antigenic determinant on an antigen to which the paratope of an antibody binds.

Epitopic determinants usually consist of chemically active surface groupings of molecules such as amino acids or carbohydrate side chains and usually have specific three dimensional structural characteristics, as well as specific charge characteristics.

The term “antibody” as used in this invention includes intact molecules as well as functional fragments thereof, such as Fab, F(ab′)2, and Fv that are capable of binding to macrophages. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)2, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Antibody fragments according to some embodiments of the invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

Anti SYNJ2 are commercially available. Examples of suppliers of anti human SYNJ2 monoclonal antibodies include, but are not limited to, Amsbio, Atlas Antibodies, AbD Serotec, United States Biological, antibodies-online com, Genway, Proteintech Group and more. Antibodies of the invention are rendered non-immunogenic for therapeutic applications.

Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

Downregulation of SYNJ2 can be also achieved by RNA silencing. As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene. RNA silencing has been observed in many types of organisms, including plants, animals, and fungi.

As used herein, the term “RNA silencing agent” refers to an RNA which is capable of specifically inhibiting or “silencing” the expression of a target gene. In certain embodiments, the RNA silencing agent is capable of preventing complete processing (e.g, the full translation and/or expression) of an mRNA molecule through a post-transcriptional silencing mechanism. RNA silencing agents include noncoding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs. In one embodiment, the RNA silencing agent is capable of inducing RNA interference. In another embodiment, the RNA silencing agent is capable of mediating translational repression.

According to an embodiment of the invention, the RNA silencing agent is specific to the target RNA (e.g., SYNJ2) and does not cross inhibit or silence a gene or a splice variant which exhibits 99% or less global homology to the target gene, e.g., less than 98%, 97%, 96%, 95%, 94%, 93%, 92%, 91%, 90%, 89%, 88%, 87%, 86%, 85%, 84%, 83%, 82%, 81% global homology to the target gene.

RNA interference refers to the process of sequence-specific post-transcriptional gene silencing in animals mediated by short interfering RNAs (siRNAs). The corresponding process in plants is commonly referred to as post-transcriptional gene silencing or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla. Such protection from foreign gene expression may have evolved in response to the production of double-stranded RNAs (dsRNAs) derived from viral infection or from the random integration of transposon elements into a host genome via a cellular response that specifically destroys homologous single-stranded RNA or viral genomic RNA.

The presence of long dsRNAs in cells stimulates the activity of a ribonuclease III enzyme referred to as dicer. Dicer is involved in the processing of the dsRNA into short pieces of dsRNA known as short interfering RNAs (siRNAs). Short interfering RNAs derived from dicer activity are typically about 21 to about 23 nucleotides in length and comprise about 19 base pair duplexes. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex.

Accordingly, some embodiments of the invention contemplate use of dsRNA to downregulate protein expression from mRNA.

According to one embodiment, the dsRNA is greater than 30 bp. The use of long dsRNAs (i.e. dsRNA greater than 30 bp) has been very limited owing to the belief that these longer regions of double stranded RNA will result in the induction of the interferon and PKR response. However, the use of long dsRNAs can provide numerous advantages in that the cell can select the optimal silencing sequence alleviating the need to test numerous siRNAs; long dsRNAs will allow for silencing libraries to have less complexity than would be necessary for siRNAs; and, perhaps most importantly, long dsRNA could prevent viral escape mutations when used as therapeutics.

Various studies demonstrate that long dsRNAs can be used to silence gene expression without inducing the stress response or causing significant off-target effects—see for example [Strat et al., Nucleic Acids Research, 2006, Vol. 34, No. 13 3803-3810; Bhargava A et al. Brain Res. Protoc. 2004; 13:115-125; Diallo M., et al., Oligonucleotides. 2003; 13:381-392; Paddison P. J., et al., Proc. Natl Acad. Sci. USA. 2002; 99:1443-1448; Tran N., et al., FEBS Lett. 2004; 573:127-134].

In particular, the invention according to some embodiments thereof contemplates introduction of long dsRNA (over 30 base transcripts) for gene silencing in cells where the interferon pathway is not activated (e.g. embryonic cells and oocytes) see for example Billy et al., PNAS 2001, Vol 98, pages 14428-14433 and Diallo et al, Oligonucleotides, Oct. 1, 2003, 13(5): 381-392. doi:10.1089/154545703322617069.

The invention according to some embodiments thereof also contemplates introduction of long dsRNA specifically designed not to induce the interferon and PKR pathways for down-regulating gene expression. For example, Shinagwa and Ishii [Genes & Dev. 17 (11): 1340-1345, 2003] have developed a vector, named pDECAP, to express long double-strand RNA from an RNA polymerase II (Pol II) promoter. Because the transcripts from pDECAP lack both the 5′-cap structure and the 3′-poly(A) tail that facilitate ds-RNA export to the cytoplasm, long ds-RNA from pDECAP does not induce the interferon response.

Another method of evading the interferon and PKR pathways in mammalian systems is by introduction of small inhibitory RNAs (siRNAs) either via transfection or endogenous expression.

The term “siRNA” refers to small inhibitory RNA duplexes (generally between 18-30 basepairs) that induce the RNA interference (RNAi) pathway. Typically, siRNAs are chemically synthesized as 21mers with a central 19 by duplex region and symmetric 2-base 3′-overhangs on the termini, although it has been recently described that chemically synthesized RNA duplexes of 25-30 base length can have as much as a 100-fold increase in potency compared with 21mers at the same location. The observed increased potency obtained using longer RNAs in triggering RNAi is theorized to result from providing Dicer with a substrate (27mer) instead of a product (21mer) and that this improves the rate or efficiency of entry of the siRNA duplex into RISC.

It has been found that position of the 3′-overhang influences potency of a siRNA and asymmetric duplexes having a 3′-overhang on the antisense strand are generally more potent than those with the 3′-overhang on the sense strand (Rose et al., 2005). This can be attributed to asymmetrical strand loading into RISC, as the opposite efficacy patterns are observed when targeting the antisense transcript.

The strands of a double-stranded interfering RNA (e.g., an siRNA) may be connected to form a hairpin or stem-loop structure (e.g., an shRNA). Thus, as mentioned the RNA silencing agent of some embodiments of the invention may also be a short hairpin RNA (shRNA).

Examples of small interfering RNA molecules can be found in Chuang et al. (supra) SJ2-1 (coding region 1612-1633; 5′AACGTGAACGGAGGAAAGCAG, SEQ ID NO: 3), SJ2-2 (region 5419-5440 in the 3′ untranslated region; 5′CTCTTGCTGATACGCGATATT, SEQ ID NO: 4); or Rusk et al. [Curr Biol. 2003 Apr. 15; 13(8):659-63. Erratum in: Curr Biol. 2003 Sep. 30; 13(19):1746], teaching siRNA to the coding regions 1612-1633 or 4925-4946 of SYNJ2.

Other examples of siRNA sequences that successfully downregulate SYNJ2 mRNA levels include, but are not limited to GAAGAAACAUCCCUUUGAU (SEQ ID NO: 5) and GGACAGCACUGCAGGUGUU (SEQ ID NO: 6).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. Examples of oligonucleotide sequences that can be used to form the loop include 5′-UUCAAGAGA-3′ (Brummelkamp, T. R. et al. (2002) Science 296: 550) and 5′-UUUGUGUAG-3′ (Castanotto, D. et al. (2002) RNA 8:1454). It will be recognized by one of skill in the art that the resulting single chain oligonucleotide forms a stem-loop or hairpin structure comprising a double-stranded region capable of interacting with the RNAi machinery.

Examples of shRNA sequences that successfully downregulate SYNJ2 mRNA levels include, but are not limited to, CCGGCCTACGATACAAGCGACAAATCTCGAAGATTTGTCGCTTGTATCGTAG GTTTTTG (SEQ ID NO: 7); CCGGCGAGAGGAGATCATTCGGAAACTCGAGTTTCCGAATGATCTCCTCTC GTTTTTG (SEQ ID NO: 8); CCGGCCGGAAGAACAGTTTGAGCAACTCGAGTTGCTCAAACTGTTCTTCCG GTTTTTG (SEQ ID NO: 9).

Synthesis of RNA silencing agents suitable for use with some embodiments of the invention can be effected as follows. First, the SYNJ2 mRNA sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (wwwdotambiondotcom/techlib/tn/91/912dothtml).

Second, potential target sites are compared to an appropriate genomic database (e.g., human, mouse, rat etc.) using any sequence alignment software, such as the BLAST software available from the NCBI server (wwwdotncbidotnlmdotnihdotgov/BL AST/). Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

It will be appreciated that the RNA silencing agent of some embodiments of the invention need not be limited to those molecules containing only RNA, but further encompasses chemically-modified nucleotides and non-nucleotides.

In some embodiments, the RNA silencing agent provided herein can be functionally associated with a cell-penetrating peptide.” As used herein, a “cell-penetrating peptide” is a peptide that comprises a short (about 12-30 residues) amino acid sequence or functional motif that confers the energy-independent (i.e., non-endocytotic) translocation properties associated with transport of the membrane-permeable complex across the plasma and/or nuclear membranes of a cell. The cell-penetrating peptide used in the membrane-permeable complex of some embodiments of the invention preferably comprises at least one non-functional cysteine residue, which is either free or derivatized to form a disulfide link with a double-stranded ribonucleic acid that has been modified for such linkage. Representative amino acid motifs conferring such properties are listed in U.S. Pat. No. 6,348,185, the contents of which are expressly incorporated herein by reference. The cell-penetrating peptides of some embodiments of the invention preferably include, but are not limited to, penetratin, transportan, pIs1, TAT(48-60), pVEC, MTS, and MAP.

mRNAs to be targeted using RNA silencing agents include, but are not limited to, those whose expression is correlated with an undesired phenotypic trait. Exemplary mRNAs that may be targeted are those that encode truncated proteins i.e. comprise deletions. Accordingly the RNA silencing agent of some embodiments of the invention may be targeted to a bridging region on either side of the deletion. Introduction of such RNA silencing agents into a cell would cause a down-regulation of the mutated protein while leaving the non-mutated protein unaffected.

According to another embodiment the RNA silencing agent may be a miRNA.

The term “microRNA”, “miRNA”, and “miR” are synonymous and refer to a collection of non-coding single-stranded RNA molecules of about 19-28 nucleotides in length, which regulate gene expression. miRNAs are found in a wide range of organisms (virusesdotfwdarwdothumans) and have been shown to play a role in development, homeostasis, and disease etiology.

Below is a brief description of the mechanism of miRNA activity.

Genes coding for miRNAs are transcribed leading to production of an miRNA precursor known as the pri-miRNA. The pri-miRNA is typically part of a polycistronic RNA comprising multiple pri-miRNAs. The pri-miRNA may form a hairpin with a stem and loop. The stem may comprise mismatched bases.

The hairpin structure of the pri-miRNA is recognized by Drosha, which is an RNase III endonuclease. Drosha typically recognizes terminal loops in the pri-miRNA and cleaves approximately two helical turns into the stem to produce a 60-70 nucleotide precursor known as the pre-miRNA. Drosha cleaves the pri-miRNA with a staggered cut typical of RNase III endonucleases yielding a pre-miRNA stem loop with a 5′ phosphate and ˜2 nucleotide 3′ overhang. It is estimated that approximately one helical turn of stem (˜10 nucleotides) extending beyond the Drosha cleavage site is essential for efficient processing. The pre-miRNA is then actively transported from the nucleus to the cytoplasm by Ran-GTP and the export receptor Ex-portin-5.

The double-stranded stem of the pre-miRNA is then recognized by Dicer, which is also an RNase III endonuclease. Dicer may also recognize the 5′ phosphate and 3′ overhang at the base of the stem loop. Dicer then cleaves off the terminal loop two helical turns away from the base of the stem loop leaving an additional 5′ phosphate and ˜2 nucleotide 3′ overhang. The resulting siRNA-like duplex, which may comprise mismatches, comprises the mature miRNA and a similar-sized fragment known as the miRNA*. The miRNA and miRNA* may be derived from opposing arms of the pri-miRNA and pre-miRNA. MiRNA* sequences may be found in libraries of cloned miRNAs but typically at lower frequency than the miRNAs.

Although initially present as a double-stranded species with miRNA*, the miRNA eventually become incorporated as a single-stranded RNA into a ribonucleoprotein complex known as the RNA-induced silencing complex (RISC). Various proteins can form the RISC, which can lead to variability in specificity for miRNA/miRNA* duplexes, binding site of the target gene, activity of miRNA (repress or activate), and which strand of the miRNA/miRNA* duplex is loaded in to the RISC.

When the miRNA strand of the miRNA:miRNA* duplex is loaded into the RISC, the miRNA* is removed and degraded. The strand of the miRNA:miRNA* duplex that is loaded into the RISC is the strand whose 5′ end is less tightly paired. In cases where both ends of the miRNA:miRNA* have roughly equivalent 5′ pairing, both miRNA and miRNA* may have gene silencing activity.

The RISC identifies target nucleic acids based on high levels of complementarity between the miRNA and the mRNA, especially by nucleotides 2-7 of the miRNA.

A number of studies have looked at the base-pairing requirement between miRNA and its mRNA target for achieving efficient inhibition of translation (reviewed by Bartel 2004, Cell 116-281). In mammalian cells, the first 8 nucleotides of the miRNA may be important (Doench & Sharp 2004 GenesDev 2004-504). However, other parts of the microRNA may also participate in mRNA binding. Moreover, sufficient base pairing at the 3′ can compensate for insufficient pairing at the 5′ (Brennecke et al, 2005 PLoS 3-e85). Computation studies, analyzing miRNA binding on whole genomes have suggested a specific role for bases 2-7 at the 5′ of the miRNA in target binding but the role of the first nucleotide, found usually to be “A” was also recognized (Lewis et at 2005 Cell 120-15). Similarly, nucleotides 1-7 or 2-8 were used to identify and validate targets by Krek et al (2005, Nat Genet 37-495).

The target sites in the mRNA may be in the 5′ UTR, the 3′ UTR or in the coding region. Interestingly, multiple miRNAs may regulate the same mRNA target by recognizing the same or multiple sites. The presence of multiple miRNA binding sites in most genetically identified targets may indicate that the cooperative action of multiple RISCs provides the most efficient translational inhibition.

MiRNAs may direct the RISC to downregulate gene expression by either of two mechanisms: mRNA cleavage or translational repression. The miRNA may specify cleavage of the mRNA if the mRNA has a certain degree of complementarity to the miRNA. When a miRNA guides cleavage, the cut is typically between the nucleotides pairing to residues 10 and 11 of the miRNA. Alternatively, the miRNA may repress translation if the miRNA does not have the requisite degree of complementarity to the miRNA. Translational repression may be more prevalent in animals since animals may have a lower degree of complementarity between the miRNA and binding site.

It should be noted that there may be variability in the 5′ and 3′ ends of any pair of miRNA and miRNA*. This variability may be due to variability in the enzymatic processing of Drosha and Dicer with respect to the site of cleavage. Variability at the 5′ and 3′ ends of miRNA and miRNA* may also be due to mismatches in the stem structures of the pri-miRNA and pre-miRNA. The mismatches of the stem strands may lead to a population of different hairpin structures. Variability in the stem structures may also lead to variability in the products of cleavage by Drosha and Dicer.

The term “microRNA mimic” refers to synthetic non-coding RNAs that are capable of entering the RNAi pathway and regulating gene expression. miRNA mimics imitate the function of endogenous microRNAs (miRNAs) and can be designed as mature, double stranded molecules or mimic precursors (e.g., or pre-miRNAs). miRNA mimics can be comprised of modified or unmodified RNA, DNA, RNA-DNA hybrids, or alternative nucleic acid chemistries (e.g., LNAs or 2′-O,4′-C-ethylene-bridged nucleic acids (ENA)). For mature, double stranded miRNA mimics, the length of the duplex region can vary between 13-33, 18-24 or 21-23 nucleotides. The miRNA may also comprise a total of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides. The sequence of the miRNA may be the first 13-33 nucleotides of the pre-miRNA. The sequence of the miRNA may also be the last 13-33 nucleotides of the pre-miRNA.

It will be appreciated from the description provided herein above, that contacting cancer cells with a miRNA may be affected in a number of ways:

1. Transiently transfecting the cancer cells with the mature double stranded miRNA.
2. Stably, or transiently transfecting the cancer cells with an expression vector which encodes the mature miRNA.
3. Stably, or transiently transfecting the cancer cells with an expression vector which encodes the pre-miRNA. The pre-miRNA sequence may comprise from 45-90, 60-80 or 60-70 nucleotides. The sequence of the pre-miRNA may comprise a miRNA and a miRNA* as set forth herein. The sequence of the pre-miRNA may also be that of a pri-miRNA excluding from 0-160 nucleotides from the 5′ and 3′ ends of the pri-miRNA.
4. Stably, or transiently transfecting the cancer cells with an expression vector which encodes the pri-miRNA. The pri-miRNA sequence may comprise from 45-30,000, 50-25,000, 100-20,000, 1,000-1,500 or 80-100 nucleotides. The sequence of the pri-miRNA may comprise a pre-miRNA, miRNA and miRNA*, as set forth herein, and variants thereof.

Another agent capable of downregulating a SYNJ2 is a DNAzyme molecule capable of specifically cleaving an mRNA transcript or DNA sequence of the SYNJ2. DNAzymes are single-stranded polynucleotides which are capable of cleaving both single and double stranded target sequences (Breaker, R.R. and Joyce, G. Chemistry and Biology 1995; 2:655; Santoro, S. W. & Joyce, G. F. Proc. Natl, Acad. Sci. USA 1997; 943:4262) A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro, S. W. & Joyce, G. F. Proc. Natl, Acad. Sci. USA 199; for rev of DNAzymes see Khachigian, L M [Curr Opin Mol Ther 4:119-21 (2002)].

Examples of construction and amplification of synthetic, engineered DNAzymes recognizing single and double-stranded target cleavage sites have been disclosed in U.S. Pat. No. 6,326,174 to Joyce et al. DNAzymes of similar design directed against the human Urokinase receptor were recently observed to inhibit Urokinase receptor expression, and successfully inhibit colon cancer cell metastasis in vivo (Itoh et al, 20002, Abstract 409, Ann Meeting Am Soc Gen Ther wwwdotasgtdotorg). In another application, DNAzymes complementary to bcr-ab1 oncogenes were successful in inhibiting the oncogenes expression in leukemia cells, and lessening relapse rates in autologous bone marrow transplant in cases of CML and ALL.

Downregulation of a SYNJ2 can also be effected by using an antisense polynucleotide capable of specifically hybridizing with an mRNA transcript encoding the SYNJ2.

Design of antisense molecules which can be used to efficiently downregulate a SYNJ2 must be effected while considering two aspects important to the antisense approach. The first aspect is delivery of the oligonucleotide into the cytoplasm of the appropriate cells, while the second aspect is design of an oligonucleotide which specifically binds the designated mRNA within cells in a way which inhibits translation thereof.

The prior art teaches of a number of delivery strategies which can be used to efficiently deliver oligonucleotides into a wide variety of cell types [see, for example, Luft J Mol Med 76: 75-6 (1998); Kronenwett et al. Blood 91: 852-62 (1998); Rajur et al. Bioconjug Chem 8: 935-40 (1997); Lavigne et al. Biochem Biophys Res Commun 237: 566-71 (1997) and Aoki et al. (1997) Biochem Biophys Res Commun 231: 540-5 (1997)].

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al. Biotechnol Bioeng 65: 1-9 (1999)].

Such algorithms have been successfully used to implement an antisense approach in cells. For example, the algorithm developed by Walton et al. enabled scientists to successfully design antisense oligonucleotides for rabbit beta-globin (RBG) and mouse tumor necrosis factor-alpha (TNF alpha) transcripts. The same research group has more recently reported that the antisense activity of rationally selected oligonucleotides against three model target mRNAs (human lactate dehydrogenase A and B and rat gp130) in cell culture as evaluated by a kinetic PCR technique proved effective in almost all cases, including tests against three different targets in two cell types with phosphodiester and phosphorothioate oligonucleotide chemistries.

In addition, several approaches for designing and predicting efficiency of specific oligonucleotides using an in vitro system were also published (Matveeva et al., Nature Biotechnology 16: 1374-1375 (1998)].

For example, a suitable antisense oligonucleotides targeted against the SYNJ2 mRNA (which is coding for the SYNJ2 protein) would be of the following sequences: CCCTTTGTCTGCCACCTCCT (SEQ ID NO: 10), ACCCATCTTGCTCTCTCCC (SEQ ID NO: 11) and TCTTCCTCCACCACAGCACC (SEQ ID NO: 12).

Several clinical trials have demonstrated safety, feasibility and activity of antisense oligonucleotides. For example, antisense oligonucleotides suitable for the treatment of cancer have been successfully used [Holmund et al., Curr Opin Mol Ther 1:372-85 (1999)], while treatment of hematological malignancies via antisense oligonucleotides targeting c-myb gene, p53 and Bcl-2 had entered clinical trials and had been shown to be tolerated by patients [Gerwitz Curr Opin Mol Ther 1:297-306 (1999)].

More recently, antisense-mediated suppression of human heparanase gene expression has been reported to inhibit pleural dissemination of human cancer cells in a mouse model [Uno et al., Cancer Res 61:7855-60 (2001)].

Thus, the current consensus is that recent developments in the field of antisense technology which, as described above, have led to the generation of highly accurate antisense design algorithms and a wide variety of oligonucleotide delivery systems, enable an ordinarily skilled artisan to design and implement antisense approaches suitable for downregulating expression of known sequences without having to resort to undue trial and error experimentation.

Another agent capable of downregulating a SYNJ2 is a ribozyme molecule capable of specifically cleaving an mRNA transcript encoding a SYNJ2. Ribozymes are being increasingly used for the sequence-specific inhibition of gene expression by the cleavage of mRNAs encoding proteins of interest [Welch et al., Curr Opin Biotechnol. 9:486-96 (1998)]. The possibility of designing ribozymes to cleave any specific target RNA has rendered them valuable tools in both basic research and therapeutic applications. In the therapeutics area, ribozymes have been exploited to target viral RNAs in infectious diseases, dominant oncogenes in cancers and specific somatic mutations in genetic disorders [Welch et al., Clin Diagn Virol. 10:163-71 (1998)]. Most notably, several ribozyme gene therapy protocols for HIV patients are already in Phase 1 trials. More recently, ribozymes have been used for transgenic animal research, gene target validation and pathway elucidation. Several ribozymes are in various stages of clinical trials. ANGIOZYME was the first chemically synthesized ribozyme to be studied in human clinical trials. ANGIOZYME specifically inhibits formation of the VEGF-r (Vascular Endothelial Growth Factor receptor), a key component in the angiogenesis pathway. Ribozyme Pharmaceuticals, Inc., as well as other firms have demonstrated the importance of anti-angiogenesis therapeutics in animal models. HEPTAZYME, a ribozyme designed to selectively destroy Hepatitis C Virus (HCV) RNA, was found effective in decreasing Hepatitis C viral RNA in cell culture assays (Ribozyme Pharmaceuticals, Incorporated—WEB home page). Another agent capable of downregulating SYNJ2 would be any molecule which binds to and/or cleaves SYNJ2.

The present teachings unveiled a general mechanism underlying the roles of SYNJ2 in cellular motility.

The principles are outlined in FIG. 15. Accordingly, a key event entails EGF-induced up-regulation of SYNJ2, and consequent depletion of three phosphoinositides: PI(4,5)P₂, PI(3,4,5)P₃ and PI(3,5)P₂. SYNJ2-mediated PI(4,5)P₂ dephosphorylation is paralleled by degradation of PI(4,5)P₂ by phospholipase C-gamma, and phosphorylation by PI3K, which generates PI(3,4,5)P₃. Collectively, stimulation of the three enzymes by EGF dissociates a group of PI(4,5)P₂ binders from the plasma membrane, and also generates PI(4,5)P₂-devoid endocytic vesicles. Concurrently, SYNJ2 converts PI(3,4,5)P₃ into PI(3,4)P₂, which is essential for invadopodia formation. Once in place, PI(3,4)P₂ binds TKS5 and nucleates a Dynamin and Cortactin-centered complex that enables Cofilin to generate actin barbed ends within invadopodia. According to the present results, SYNJ2 is involved also in the next invadopodia maturation steps, namely secretion of MMPs and delivery of MT1-MMP and other surface molecules, such as CD44. In a similar way, SYNJ2 controls delivery of EGFRs and integrin to the leading edge, and likely activates Cofillin, a pivotal event dictating formation of lamellipodial protrusions.

These findings can be harnessed towards identifying SYNJ2 inhibitors, which are putative inhibitors of tumor metastasis.

Thus, according to an aspect of the invention there is provided a method of identifying a putative inhibitor of tumor metastasis, the method comprising analyzing SYNJ2-mediated processing of PI(3,4,5)P₃ to PI(3,4)P₂ in the presence of a test agent, wherein a decreased processing of PI(3,4,5)P₃ to PI(3,4)P₂ in said presence of said test agent as compared to same in an absence thereof is indicative of a putative inhibitor of tumor metastasis.

The test agent may be a biomolecule (protein e.g., a peptide or an antibody, nucleic acid molecule e.g., silencing agent, a carbohydrate, a lipid or a combination of same) or a small molecule (e.g., chemical).

The method can be effected in vivo or in vitro. The latter may be implemented in a cellular system or using a cell-free system.

An exemplary assay, involves analyzing SYNJ2-mediated processing of PI(3,4,5)P₃ to PI(3,4)P₂ by a competition assay.

Accordingly, the competition assay tests displacement of a PI(3,4)P₂ binding domain from a complex comprising the PI(3,4)P₂ binding domain bound to PI(3,4)P₂.

According to an exemplary embodiment, a fluorescence polarization competitive assay is employed. The assay relies on the principle that once molecules bind a sequestering bigger element (e.g., a protein) their movement in space is significantly decreased. This phenomenon can be detected and measured using a fluorescent probe, which allows assaying fluorescent polarization following measurements from the parallel and perpendicular planes of the sample. Accordingly, unbound fluorescent. molecules in solution give rise to very low polarization readings, but when a detector (e.g., a binding protein) that binds (sequesters) these molecules is added to the solution, the fluorescent molecules are stabilized in a confined composition that increases the polarization readings in the solution.

For example, the assay may comprise a PI(3,4)P₂ binding domain (e.g.; PH-domain e.g.; Tapp1 PH domain, SEQ ID NOs: 15-16) and a fluorescent PI(3,4)P₂, along with a recombinant SYNJ2 and its non-fluorescent substrate, PI(3,4,5)P₃. The product of SYNJ2'S catalytic activity displaces the fluorescent PI(3,4)P₂, thus decreasing fluorescence polarization.

According to specific embodiments a commercial 5′ PI(3,4,5)P3 Phosphatase Activity Fluorescence Polarization Assay is used (e.g.; Echelon Bioscience, cat. no. K-1400).

According to specific embodiments, a reaction mix comprising SYNJ2 and PI(3,4,5)P3, as a substrate, is incubated under conditions which allow catalytic activity (dephosphorylation) of SYNJ2, with or without a test agent is prepared. The test agent may be, for example, a small molecule, a nucleic acid molecule, a peptide, an antibody, a carbohydrate or a combination of same. Following incubation, the solution containing the PI(3,4)P2 products is mixed with a mixture of PI(3,4)P2 binding protein (e.g.; PH-domain of Tapp1, SEQ ID NO: 15) and a fluorescent PI(3,4)P2 and fluorescence polarization is measured. The polarization values measured in this assay decreases as the bound fluorescent PI(3,4)P2 molecules are being displaced by un-labeled PI(3,4)P2 produced by the enzymatic activity of SYNJ2 and the amount of unbound fluorescent PI(3,4)P2 molecules in the solution increases. In the case where the value of fluorescence polarization in the presence of the test agent increases in comparison to the value in the absence of the test agent, the test agent is a putative SYNJ2 inhibitor.

Once identified, the functionality of the test agent as an anti metastatic drug is further substantiated using relevant assays, such as the gelatin-zymography assay, transwell assay and test animals as exemplified further below.

Using this methodology the present inventors have identified a number of small molecules which can be used as SYNJ2 inhibitors according to some embodiments of the present invention. These molecules are depicted in FIG. 19 and shown in Table 2 of Example 10, hereinbelow.

As mentioned, the inhibitor of SYNJ2 is administered in addition to an inhibitor of a cell surface receptor associated with an onset or progression of cancer. According to an embodiment of the invention, the receptor is an oncogene.

Examples of receptors which may be targeted according to the present teachings are receptor tyrosine kinases such as those EGFR, PDGFR, VEGFR, FGFR and ErbB-2.

Other surface molecules which can be targeted include integrins matrix metallo proteinases (MMPs), dynamin, TKS5 and CD44.

Inhibitors of cell surface molecules are well known in the art. A non-limiting list of such inhibitors is provided infra.

Thus for example, the identification of EGFR as an oncogene has led to the development of anticancer therapeutics directed against EGFR.

Cetuximab and panitumumab are examples of monoclonal antibody inhibitors. Other monoclonals in clinical development are zalutumumab, nimotuzumab, and matuzumab. The monoclonal antibodies block the extracellular ligand binding domain. With the binding site blocked, signal molecules can no longer attach there and activate the tyrosine kinase.

Another method is using small molecules to inhibit the EGFR tyrosine kinase, which is on the cytoplasmic side of the receptor. Without kinase activity, EGFR is unable to activate itself, which is a prerequisite for binding of downstream adaptor proteins. Ostensibly by halting the signaling cascade in cells that rely on this pathway for growth, tumor proliferation and migration is diminished. Gefitinib, erlotinib, and lapatinib (mixed EGFR and ERBB2 inhibitor) are examples of small molecule kinase inhibitors. Other examples include, Iressa and Tarceva directly target the EGFR.

HER2 is the target of the monoclonal antibody trastuzumab (marketed as Herceptin). Trastuzumab is effective only in cancers where HER2 is over-expressed. Another monoclonal antibody, Pertuzumab, which inhibits dimerization of HER2 and HER3 receptors, was approved by the FDA for use in combination with trastuzumab in June 2012.

Additionally, NeuVax (Galena Biopharma) is a peptide-based immunotherapy that directs “killer” T cells to target and destroy cancer cells that express HER2.

The expression of HER2 is regulated by signaling through estrogen receptors. Estradiol and tamoxifen acting through the estrogen receptor down-regulate the expression of HER2.

Examples of antibodies which can be used according to the present teachings are listed below and are by no way meant to be limiting.

TABLE 1

| | Brand | Approval | | | |
|---|---|---|---|---|---|
| Antibody | name | date | Type | Target | Approved treatment(s) |
| Alemtuzumab | Campath | 2001 | humanized | CD52 | Chronic lymphocytic |
| | | | | | leukemia |
| Bevacizumab | Avastin | 2004 | humanized | vascular | colorectal cancer |
| | | | | endothelial | |
| | | | | growth factor | |
| Brentuximab | Adcetris | 2011 | chimeric | CD30 | Hodgkin lymphoma, |
| vedotin | | | | | Anaplastic large-cell |
| | | | | | lymphoma |
| Cetuximab | Erbitux | 2004 | chimeric | epidermal | colorectal cancer |
| | | | | growth factor | |
| | | | | receptor | |
| Gemtuzumab | Mylotarg | 2000 | humanized | CD33 | acute myelogenous |
| ozogamicin | | | | | leukemia (with |
| | | | | | calicheamicin) |
| Ibritumomab | Zevalin | 2002 | murine | CD20 | non-Hodgkin |
| tiuxetan | | | | | lymphoma (with |
| | | | | | yttrium-90 or indium- |
| | | | | | 111) |
| Panitumumab | Vectibix | 2006 | human | epidermal | colorectal cancer |
| | | | | growth factor | |
| | | | | receptor | |
| Rituximab | Rituxan, | 1997 | chimeric | CD20 | non-Hodgkin |
| | Mabthera | | | | lymphoma |
| Trastuzumab | Herceptin | 1998 | humanized | ErbB2 | breast cancer |

The inhibitors of the SYNJ2 and optionally the inhibitor of the cell surface receptor as described herein can be administered to the subject per se or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the inhibitor of the SYNJ2 (and optionally the inhibitor of the cell surface receptor) accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

The term “tissue” refers to part of an organism consisting of cells designed to perform a function or functions. Examples include, but are not limited to, brain tissue, retina, skin tissue, hepatic tissue, pancreatic tissue, bone, cartilage, connective tissue, blood tissue, muscle tissue, cardiac tissue brain tissue, vascular tissue, renal tissue, pulmonary tissue, gonadal tissue, hematopoietic tissue.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (SYNJ2 inhibitor) effective to prevent, alleviate or ameliorate symptoms of a disorder (e.g., cancer or metastatic cancer) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fing1, et al., 1975, in “The Pharmacological Basis of Therapeutics”, Ch. 1 p.1).

Dosage amount and interval may be adjusted individually to provide SYNJ2 inhibitor levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

In line with the contribution of SYNJ2 to cell migration, the present inventors observed a significant up-regulation of SYNJ2 mRNA and protein levels in aggressive subtypes of cancer, suggesting that SYNJ2 may be used as a prognostic marker.

Thus, according to an aspect of the invention there is provided a method of prognosing cancer in a subject in need thereof, the method comprising determining a level or activity of SYNJ2 in a cancer cell of the subject, wherein an upregulation in said level of activity of said SYNJ2 in said cancer cell of the subject compared to same in a cell of an unaffected control sample, is indicative of a poor prognosis.

As used herein the term “prognosing” refers to determining the outcome of the disease (cancer).

As used herein “poor prognosis” refers to increased risk of recurrence of the disease and/or increased risk of death due to the disease.

As used herein the term “level” refers to expression level at the DNA (gene amplification), RNA or protein.

As used herein “SYNJ2 activity” refers to primarily to its phosphatase activity i.e., converting PI(3,4,5)P₃ to PI(3,4)P₂

According to a specific embodiment, the activity is assayed using an in vitro activity assay.

In Vitro Activity Assays:

In these methods the activity of a particular enzyme (in this case phosphatase) is measured in a protein mixture extracted from the cells. The activity can be measured in a spectrophotometer well using colorimetric methods or can be measured in a non-denaturing acrylamide gel (i.e., activity gel). Following electrophoresis the gel is soaked in a solution containing a substrate and colorimetric reagents. The resulting stained band corresponds to the enzymatic activity of the protein of interest. If well calibrated and within the linear range of response, the amount of enzyme present in the sample is proportional to the amount of color produced. An enzyme standard is generally employed to improve quantitative accuracy.

A specific assay for SYNJ2 is described above, where the PI(3,4,5)P₃ to PI(3,4)P₂ conversion activity is tested.

Methods of Detecting Expression and/or Activity of Proteins

Protein expression of SYNJ2 can be determined using methods known in the arts.

Enzyme Linked Immunosorbent Assay (ELISA):

This method involves fixation of a sample (e.g., fixed cells or a proteinaceous solution) containing a protein substrate to a surface such as a well of a microtiter plate. A substrate specific antibody coupled to an enzyme is applied and allowed to bind to the substrate. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Western Blot:

This method involves separation of a substrate from other protein by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radiolabeled or enzyme linked as described hereinabove. Detection may be by autoradiography, colorimetric reaction or chemiluminescence. This method allows both quantitation of an amount of substrate and determination of its identity by a relative position on the membrane which is indicative of a migration distance in the acrylamide gel during electrophoresis.

Radio-Immunoassay (RIA):

In one version, this method involves precipitation of the desired protein (i.e., the substrate) with a specific antibody and radiolabeled antibody binding protein (e.g., protein A labeled with I¹²⁵) immobilized on a precipitable carrier such as agarose beads. The number of counts in the precipitated pellet is proportional to the amount of substrate.

In an alternate version of the RIA, a labeled substrate and an unlabelled antibody binding protein are employed. A sample containing an unknown amount of substrate is added in varying amounts. The decrease in precipitated counts from the labeled substrate is proportional to the amount of substrate in the added sample.

Fluorescence Activated Cell Sorting (FACS):

This method involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

Immunohistochemical Analysis:

This method involves detection of a substrate in situ in fixed cells by substrate specific antibodies. The substrate specific antibodies may be enzyme linked or linked to fluorophores. Detection is by microscopy and subjective or automatic evaluation. If enzyme linked antibodies are employed, a colorimetric reaction may be required. It will be appreciated that immunohistochemistry is often followed by counterstaining of the cell nuclei using for example Hematoxyline or Giemsa stain.

In Situ Activity Assay:

According to this method, a chromogenic substrate is applied on the cells containing an active enzyme and the enzyme catalyzes a reaction in which the substrate is decomposed to produce a chromogenic product visible by a light or a fluorescent microscope.

Alternatively or additionally, the level of SYNJ2 is detected at the RNA level using methods which are well known in the arts and some are described infra.

Methods of Detecting the Expression Level of RNA

The expression level of the RNA in the cells of some embodiments of the invention can be determined using methods known in the arts.

Northern Blot Analysis:

This method involves the detection of a particular RNA in a mixture of RNAs. An RNA sample is denatured by treatment with an agent (e.g., formaldehyde) that prevents hydrogen bonding between base pairs, ensuring that all the RNA molecules have an unfolded, linear conformation. The individual RNA molecules are then separated according to size by gel electrophoresis and transferred to a nitrocellulose or a nylon-based membrane to which the denatured RNAs adhere. The membrane is then exposed to labeled DNA probes. Probes may be labeled using radio-isotopes or enzyme linked nucleotides. Detection may be using autoradiography, colorimetric reaction or chemiluminescence. This method allows both quantitation of an amount of particular RNA molecules and determination of its identity by a relative position on the membrane which is indicative of a migration distance in the gel during electrophoresis.

RT-PCR Analysis:

This method uses PCR amplification of relatively rare RNAs molecules. First, RNA molecules are purified from the cells and converted into complementary DNA (cDNA) using a reverse transcriptase enzyme (such as an MMLV-RT) and primers such as, oligo dT, random hexamers or gene specific primers. Then by applying gene specific primers and Taq DNA polymerase, a PCR amplification reaction is carried out in a PCR machine. Those of skills in the art are capable of selecting the length and sequence of the gene specific primers and the PCR conditions (i.e., annealing temperatures, number of cycles and the like) which are suitable for detecting specific RNA molecules. It will be appreciated that a semi-quantitative RT-PCR reaction can be employed by adjusting the number of PCR cycles and comparing the amplification product to known controls.

RNA In Situ Hybridization Stain:

In this method DNA or RNA probes are attached to the RNA molecules present in the cells. Generally, the cells are first fixed to microscopic slides to preserve the cellular structure and to prevent the RNA molecules from being degraded and then are subjected to hybridization buffer containing the labeled probe. The hybridization buffer includes reagents such as formamide and salts (e.g., sodium chloride and sodium citrate) which enable specific hybridization of the DNA or RNA probes with their target mRNA molecules in situ while avoiding non-specific binding of probe. Those of skills in the art are capable of adjusting the hybridization conditions (i.e., temperature, concentration of salts and formamide and the like) to specific probes and types of cells. Following hybridization, any unbound probe is washed off and the bound probe is detected using known methods. For example, if a radio-labeled probe is used, then the slide is subjected to a photographic emulsion which reveals signals generated using radio-labeled probes; if the probe was labeled with an enzyme then the enzyme-specific substrate is added for the formation of a colorimetric reaction; if the probe is labeled using a fluorescent label, then the bound probe is revealed using a fluorescent microscope; if the probe is labeled using a tag (e.g., digoxigenin, biotin, and the like) then the bound probe can be detected following interaction with a tag-specific antibody which can be detected using known methods.

In Situ RT-PCR Stain:

This method is described in Nuovo G J, et al. [Intracellular localization of polymerase chain reaction (PCR)-amplified hepatitis C cDNA. Am J Surg Pathol. 1993, 17: 683-90] and Komminoth P, et al. [Evaluation of methods for hepatitis C virus detection in archival liver biopsies. Comparison of histology, immunohistochemistry, in situ hybridization, reverse transcriptase polymerase chain reaction (RT-PCR) and in situ RT-PCR. Pathol Res Pract. 1994, 190: 1017-25]. Briefly, the RT-PCR reaction is performed on fixed cells by incorporating labeled nucleotides to the PCR reaction. The reaction is carried on using a specific in situ RT-PCR apparatus such as the laser-capture microdissection PixCell I LCM system available from Arcturus Engineering (Mountainview, Calif.).

DNA Microarrays/DNA Chips:

The expression of thousands of genes may be analyzed simultaneously using DNA microarrays, allowing analysis of the complete transcriptional program of an organism during specific developmental processes or physiological responses. DNA microarrays consist of thousands of individual gene sequences attached to closely packed areas on the surface of a support such as a glass microscope slide. Various methods have been developed for preparing DNA microarrays. In one method, an approximately 1 kilobase segment of the coding region of each gene for analysis is individually PCR amplified. A robotic apparatus is employed to apply each amplified DNA sample to closely spaced zones on the surface of a glass microscope slide, which is subsequently processed by thermal and chemical treatment to bind the DNA sequences to the surface of the support and denature them. Typically, such arrays are about 2×2 cm and contain about individual nucleic acids 6000 spots. In a variant of the technique, multiple DNA oligonucleotides, usually 20 nucleotides in length, are synthesized from an initial nucleotide that is covalently bound to the surface of a support, such that tens of thousands of identical oligonucleotides are synthesized in a small square zone on the surface of the support. Multiple oligonucleotide sequences from a single gene are synthesized in neighboring regions of the slide for analysis of expression of that gene. Hence, thousands of genes can be represented on one glass slide. Such arrays of synthetic oligonucleotides may be referred to in the art as “DNA chips”, as opposed to “DNA microarrays”, as described above [Lodish et al. (eds.). Chapter 7.8: DNA Microarrays: Analyzing Genome-Wide Expression. In: Molecular Cell Biology, 4th ed., W. H. Freeman, New York. (2000)].

The prognosis can be substantiated by using Gold standard methods e.g., imaging methods, biopsy sampling, marker expression, immunohistochemistry and the like.

The following is a specific example for breast cancer but is by no way meant to be limiting. The prognosis of breast cancer is usually determined by the disease stage (TNM stage) after surgery that assesses the size of tumor (T), the status of metastasis to adjacent lymph nodes (N), and the presence or absence of distant metastasis to other organs (M). The prognosis of patients classified according to TNM stage is different even in the same stage. In other words, in the same stage of breast cancer, the prognosis can be determined by the expression of estrogen or progesterone receptor (ER or PR) and the over-expression of HER2 protein or the amplification of the gene.

The agents of some embodiments of the invention which are described hereinabove for detecting the SYNJ2 may be included in a diagnostic kit/article of manufacture preferably along with appropriate instructions for use and labels indicating FDA approval for use in diagnosing and/or assessing cancer stage and/or prognosis.

Such a kit can include, for example, at least one container including at least one of the above described diagnostic agents (e.g., anti SYNJ2 antibody e.g., along with anti-HER2 and/or anti ER or oligonucleotide probes/primers for these targets) and an imaging reagent packed in another container (e.g., enzymes, secondary antibodies, buffers, chromogenic substrates, fluorogenic material). The kit may also include appropriate buffers and preservatives for improving the shelf-life of the kit.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof. Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Materials and Methods

Cell Migration, Invasion and Chemotaxis Assays

Cells were plated in triplicates in the upper compartment of a Transwell tray (BD Bioscience), and allowed to migrate through the intervening membrane for 18 hours. Thereafter, cells were fixed in paraformaldehyde (3%), permeabilized in Triton X-100 (0.05%) and stained with methyl violet (0.02%). Non-migrating cells, growing on the upper side of the filter, were removed and migrated cells photographed. Invasion assays were performed using BioCoat Matrigel Chambers. For chemotaxis chambers from ibidi (München, Germany) and time-lapse imaging were used. The positions of cell nuclei were tracked using ImageJ.

Phosphoinositide Analyses

Cells were incubated for 30 minutes in inositol-free medium, which was changed to medium supplemented with both [³H]-inositol and dialyzed serum (10%). Cells were cultured for three days, rinsed and extracted in 1M HCl followed by 1M Methanol. The cells were then scraped and extracted in chloroform, and then in methanol:0.1M EDTA pH8.0, and the organic phase evaporated. Thereafter, extracts were de-acetylated, separated by anionic-exchange HPLC (Agilent 1200) using two partisphere SAX columns (Whatman) in tandem, and a four-step gradient of ammonium phosphate pH 6.0. The radiolabelled eluate was detected by an online flow scintillation analyzer and quantified using ProFSA software (Perkin-Elmer).

Gelatin Zymography

To detect MMP-2 activity, biological samples were separated electrophoretically on 10% polyacrylamide/0.1% gelatin-embedded gels. The gels were then washed in 2.5% Triton X-100, and incubated at 37° C. for 36 hours in 50 mM Tris-HCl (pH 7.5), containing 0.2 M NaCl, 5 mM CaCl₂, 1 μM ZnCl₂, 0.02% Brij 35, and 1 mM p-aminophenylmercuric acetate.

Metastasis Tests in Animals

Female CB-17 SCID mice (Harlan Laboratories, Haslett, Mich.; 15 per group) were implanted in the fat pad with MDA-MB-231 cells (1.4×10⁶ cells/mouse). Two and six weeks post implantation, mice were anesthetized, tumor sizes were measured and metastases in lymph nodes were visualized using a fluorescent binocular. For lung metastases, mice were sacrificed, lungs were removed, washed, and images were acquired using a fluorescent binocular. Two-sided Fischer's exact test was used for analysis of lymph node metastasis. Tumor growth measurements used the Exact-sig [2×1-tailed]) Mann-Whitney test.

Reagents

Unless indicated, Human recombinant growth factors and other materials were purchased from Sigma (St. Louis, Mo., USA). Radioactive materials and a chemiluminescence kit for immunoblotting were obtained from Amersham (Buckinghamshire, UK). The EGFR-kinase inhibitor AG1478, MEK inhibitor U0126 and the PI3K inhibitor Wortmannin were from Calbiochem (San Diego, Calif.). Plates for wound-healing assays were from ibidi (Munich, Germany). 35-mm glass-bottom dishes for time-lapse imaging were purchased from MaTek (Ashland, Mass.). Murine monoclonal antibody (mAb) 111.6 to the EGF-receptor was generated in our laboratory. Anti-EGFR for western blot analysis was from Alexis (Lausen, Switzerland). Anti Ras-GAP and anti-AKT antibodies were from Santa Cruz Biotechnology (Santa Cruz, Calif.). Anti-EEA1, anti-Rab5, anti-Rab4, and anti Rac1 were from BD Transduction Laboratories (Franklin Lakes, N.J.). Anti-SYNJ2 mAb was from Abnova (Taipei, Taiwan). The following secondary antibodies were used: goat anti-mouse IgG and goat anti-rabbit IgG antibodies conjugated to Horseradish peroxidase (HRP) were purchased from Jackson ImmunoResearch Laboratories (Bar Harbor, Me.). Texas-red transferrin, goat anti mouse Alexa-488, Alexa-555 and Alexa-647 secondary antibodies were from Invitrogen (Carlsbad, Calif.).

siRNA control was from “Thermo scientific Dharmacon” cat. D-001810-10-05; siRNA sequence against SYNJ2 is as set forth in SEQ ID NO: 6—GGACAGCACUGCAGGUGUU; all shRNA were from SIGMA Israel: shRNA control—cat. SHC002; shRNA sequences against SYNJ2 used is CCGGCCGGAAGAACAGTTTGAGCAACTCGAGTTGCTCAAACTGTTCTTCCG GTTTTTG (SEQ ID NO: 9).

Cell Lines and Transfections

MCF10A cells were grown in DMEM:F12 (1:1) medium supplemented with antibiotics, insulin (10 μg/mL), cholera toxin (0.1 μg/mL), hydrocortisone (0.5 μg/mL), heat-inactivated horse serum (5% vol/vol), and EGF (10 ng/mL). Human mammary MDA-MB-231 cells were grown in RPMI-1640 (Gibco BRL; Grand Island, N.Y.) supplemented with 10% heat-inactivated fetal calf serum (Gibco), 1 mM sodium pyruvate and a penicillin-streptomycin mixture (100 unit/ml; 0.1 mg/ml; Beit Haemek, Israel). The MDA-MB-231-RFP stable cell-line was a kind gift from Prof. Hadasa Degani (The Weizmann Institute of Science, Israel). Plasmid transfections were performed using Fugen-HD according to the manufacture's guidelines (Roche, Mannheim, Germany). Alternatively, for transient mRNA knockdown experiments using siRNA oligonucleotides, cells were transfected with Oligofectamine (Invitrogen).

Lentiviral Vectors and Virus Production

Non-targeted shRNA hairpins (control) and hairpins directed against human SYNJ2 were produced in HEK-293T cells following the manufacture's guidelines (Sigma). Target cells were infected with shRNA-encoding lentiviruses supplemented with polybrene (8 μg/mL), and cultured in the presence of puromycin (2 μg/mL) for 4 days. Stable gene-specific delivery of human SYNJ2 was performed using the ViraPower lentiviral expression system (Invitrogen), following the manufacture's guidelines.

Immunofluorescence and Image Processing

Cells were grown on fibronectin-coated cover slips for 48 hours. Following treatments, cells were washed, permeabilized using 0.02% Triton X-100 and 3% paraformaldehyde, and fixed for 20 minutes. Confocal microscopy was performed using either a Zeiss LSM-710 microscope, or a spinning disk microscope (Zeiss 100×, NA 1.45; Yokogawa CSU-22; Zeiss fully automated, inverted 200 M; Photometrics HQ-CCD camera) and solid state lasers (473, 561 and 660 nm, exposure times: 0.25-1 sec), under the command of Slidebook™. 3D image stacks were acquired every 70-300 ms along the Z-axis by varying the position of the piezo electrically controlled stage (step size: 0.1-0.4 μm). Alternatively, live cell fluorescence microscopy was carried out using the DeltaVision system (Applied Precision, Issaqua, Wash.) and images were processed using the prism software.

Radiolabeling of EGF

Human recombinant EGF was labeled with IODOGEN as follows: EGF (5 μg) was mixed in an Iodogen-coated tube (1 mg of reagent) with Na¹²⁵I (1 mCi). Following 15 minutes of incubation at 23° C., albumin was added to a final concentration of 0.1 mg/ml, and the mixture was separated on an Excellulose GF-5 column.

Receptor Downregulation Assay

MDA-MB-231 cells were seeded in triplicates for each time point in 24-well plates, with an additional well plated for control. 48 hours later, cells were starved for 4 hours and stimulated with EGF (2 ng/ml) at 37° C. for the indicated time intervals. Subsequently, they were placed on ice, rinsed once with binding buffer (DME medium, albumin 1%, Hepes 20 mM, pH 7.5), and subjected to mild acid/salt wash (0.2 M Na Acetate buffer pH 4.5, 0.5 M NaCl) to remove surface-bound EGF. Thereafter, cells were incubated with a radiolabelled EGF for 1.5 hours at 4° C. and rinsed with binding buffer. The control well was incubated with a radiolabelled EGF and an excess of unlabelled EGF. Finally, cells were lysed with 1M NaOH, and radioactivity was determined using a γ-counter. Data represent the percentage of receptors on the cell surface relative to time 0.

Determination of Surface EGF-Receptor

Cells (2×10⁴/well) were seeded in triplicates in 24-well plates, with an additional well plated for control. Thereafter, cells were incubated with a radiolabelled EGF for 1.5 hours at 4° C. and rinsed with binding buffer. The control well was incubated with a radiolabelled EGF and an excess of unlabelled EGF. Finally, cells were lysed in 1M NaOH solution and radioactivity was determined. Data represent the percentage of receptors on the cell surface relative to control cells.

Immunoblotting Analysis

Cells were washed briefly with ice-cold saline, and scraped in a buffered detergent solution (25 mM HEPES (pH 7.5), 150 mM NaCl, 0.5% Na-deoxycholate, 1% NP-40, 0.1% SDS, 1 mM EDTA, 1 mM EGTA, 0.2 mM Na₃VO₄ and a protease inhibitor cocktail diluted at 1:1000). For equal gel loading, protein concentrations were determined by using the BCA (Pierce) reagent. Following gel electrophoresis, proteins were transferred to a nitrocellulose membrane. Membranes were blocked in TBST buffer (0.02 M Tris-Hcl (pH 7.5), 0.15 M NaCl and 0.05% Tween 20) containing 10% low-fat milk, blotted with a primary antibody for 1 hour, washed with TBST and incubated for 30 minutes with a secondary antibody conjugated to HRP.

Wound Healing (Scratch) Assays

Wound healing assays were performed according to manufacturer's protocol (iBidi, Germany). Briefly, MCF10A cells were trypsinized, re-suspended in EGF-deprived medium (7.0×10⁵ cells/mL) and 70 μl plated into each well, resulting in a confluent layer within 24 hours. Thereafter, Culture-Inserts were removed by using sterile tweezers and cells were allowed to migrate for 2 hours.

Scanning and Transmission Electron Microscopy

Cells were fixed in saline supplemented with 4% paraformaldehyde and 2% sucrose. Samples were washed and subjected to a second fixative (3% paraformaldehyde and 2.5% glutaraldehyde in 0.1 M cacodylate buffer supplemented with 1% sucrose and 5 mM CaCl₂, pH 7.4). Cells were washed in 0.1 M cacodylate buffer and post-fixed with 1% osmium tetroxide in cacodylate buffer for 1 hour. For scanning electron microscopy (SEM), the post-fixed samples were washed twice and treated with 1% tannic acid for 5 minutes followed by another wash and treatment with 1% uranyl acetate for 30 minutes. Samples were dehydrated in graded ethanol, and made conductive by sputtering with a gold-palladium film. The samples were photographed using a scanning electron microscope (Leo Supra 55/Vp Zeiss, Thornwood, N.Y.).

Receptor Recycling Assay

MDA-MB-231 cells were pre-incubated for 30 minutes at 37° C. with Alexa Fluor 488-transferrin (25 μg/ml in serum-free medium) or for 10 minutes with Alexa Fluor 488-EGF (40 ng/mL). Surface-bound ligands were detached by incubation for 30 minute at 4° C. in an acidic buffer (150 mM NaCl, 1 mM MgCl₂, 0.125 mM CaCl₂, 0.1M glycine), prior to transfer to 37° C. for the indicated time intervals, to allow for recycling of the internalized ligands. Cells were analyzed either by imaging or by FACS.

Real-Time Cell Impedance Analysis

Measurements of cell spreading and adhesion were recorded by using the RTCA-Xcelligence System (Roche Diagnostics, Mannheim, Germany). Gold microelectrode E-plates-16 were washed once in saline. Cells (2,500 per well) were first seeded and then impedance data (cell index; derived as a relative change in measured electrical impedance) was recorded in the indicated intervals. The data was analyzed using software package 1.2 provided by the manufacturer.

TAPP1-PH Domain Expression and Purification

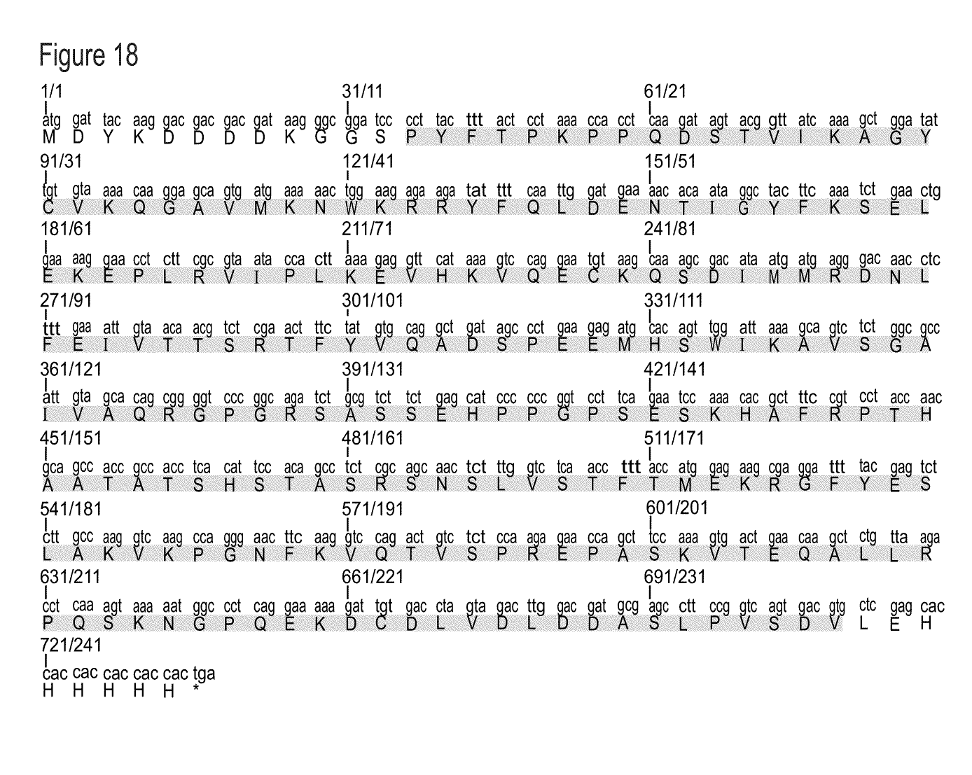

A construct encoding TAPP1-PH domain and an N-terminal Flag tag and C-terminal 6xHis tag (SEQ ID NO.: 13, FIG. 18) was cloned into pET28 plasmid and expressed in E. coli BL21(DE3) following induction with 200 μM IPTG. The bacteria were grown at 15° C. and then lysed with a cell disrupter. Cell debris was removed by centrifugation and the protein was captured on a Ni column (HisPrep FF 16/10, GE Healthcare) equilibrated with 50 mM Tris pH 8, 0.5 M NaCl and 20 mM imidazole. The protein was eluted in the same buffer containing 0.5 M Imidazole. Fractions containing the TAPP1-PH domain were injected into a size exclusion column (Hiload_{—}26/60_Superdex 75, GE Healthcare) equilibrated with buffer containing 50 mM Tris pH 8 and 100 mM NaCl. The pooled peak containing TAPP1-PH domain was diluted three fold with 20 mM sodium phosphate buffer pH 7.2 and loaded onto a cation exchange column (HiTrap_SP_FF_{—}5 ml, GE Healthcare) equilibrated with the same phosphate buffer. The pure protein was eluted from the column with a linear gradient of the phosphate buffer containing 1 M NaCl (TAPP1-PH domain elutes at 200 mM NaCl). The fractions containing the pure TAPP1-PH domain as evaluated by SDS-PAGE were pooled together and protein concentration was determined by Bradford reagent and OD₂₈₀ (extinction coefficient of 20,520) quantization. The protein was divided into aliquots, flash frozen with liquid nitrogen and stored at −80° C.

5′ Phosphatase Activity of SYNJ2

Measurements of the ability of SYNJ2 to hydrolyze the 5-phosphate from PI(3,4,5)P3 to generate PI(3,4)P2 were recorded by a competitive assay, based on fluorescence polarization as a read out. Stabilizing SOP lipid mix (×50) was prepared in a glass tube by adding 100 μl of SOPS (Avanti Inc., 50 mg/ml in chloroform) and 50 μl Cholesterol (Sigma Aldrich, 10 mg/ml in chloroform). The mix was air-dried using gentle nitrogen steam to evaporate the chloroform. The evaporated lipid mix was then re-suspended in 10 ml of 0.25 mg/ml C₁₂E₈ (Avanti Inc.) by 1 minute vortex at room temperature. A reaction mix comprising PBS, DTT, MgCl2 (all from Sigma Aldrich), SOP lipid mix (×50), full length purified SYNJ2 (OriGene, cat no. TP315160) and PI(3,4,5)P3 (Echelon Bioscience, cat no. P-3908), with or without a tested compound. Once PI(3,4,5)P3 was added, the reaction mix was incubated in 33° C. for 8 minutes to allow production of PI(3,4)P2 by SYNJ2 5′-phosphatase activity. Following incubation the of reaction was stopped by adding a detection mix comprising PBS, DTT, detector proteins (PH domain of TAPP1), SOP lipid mix (×50), fluorescently-labeled PI(3,4)P2 (Echelon Bioscience, cat no. C34M6) and EDTA (Sigma Aldrich). Fluorescence polarization was measured using an appropriate plate reader and filter set compatible with BODIPY® TMR dye (550 nm excitation/580 nm polarizing emission filters). Unlabeled PI(3,4)P2 control was purchased from Echelon Bioscience (Cat no. P-3408).

### Example 2

### EGF-Induced Elevated Expression of SYNJ2 Promotes Mammary Cell Invasion

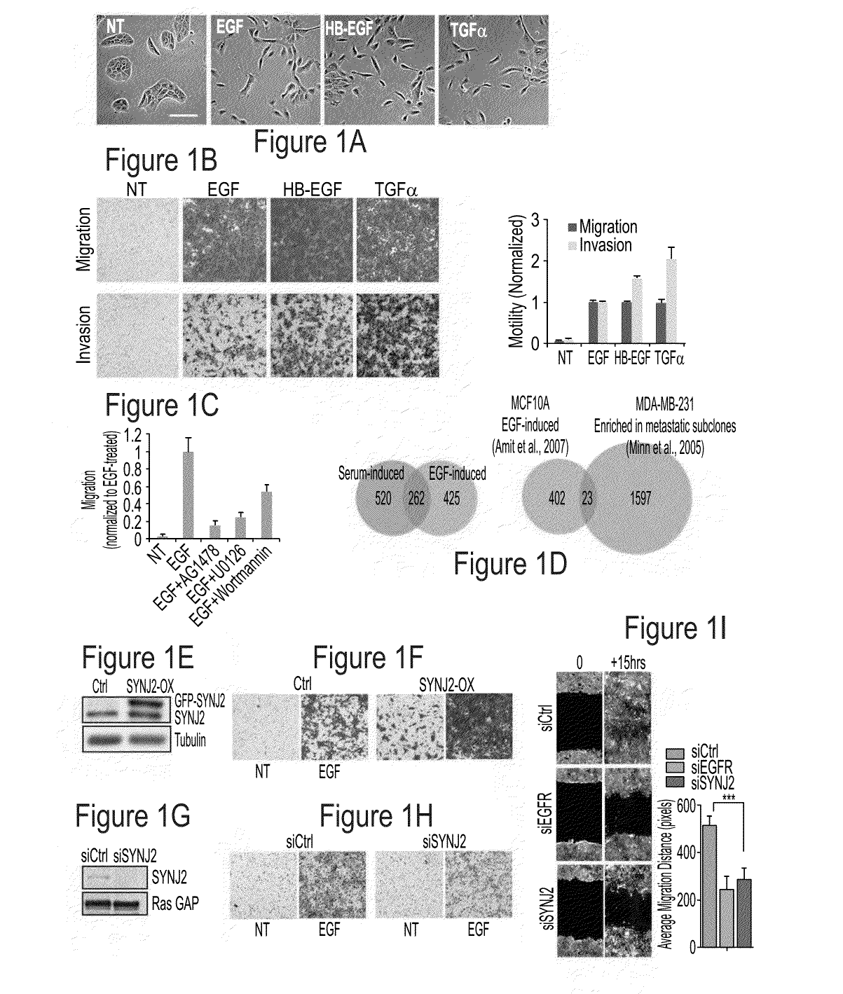

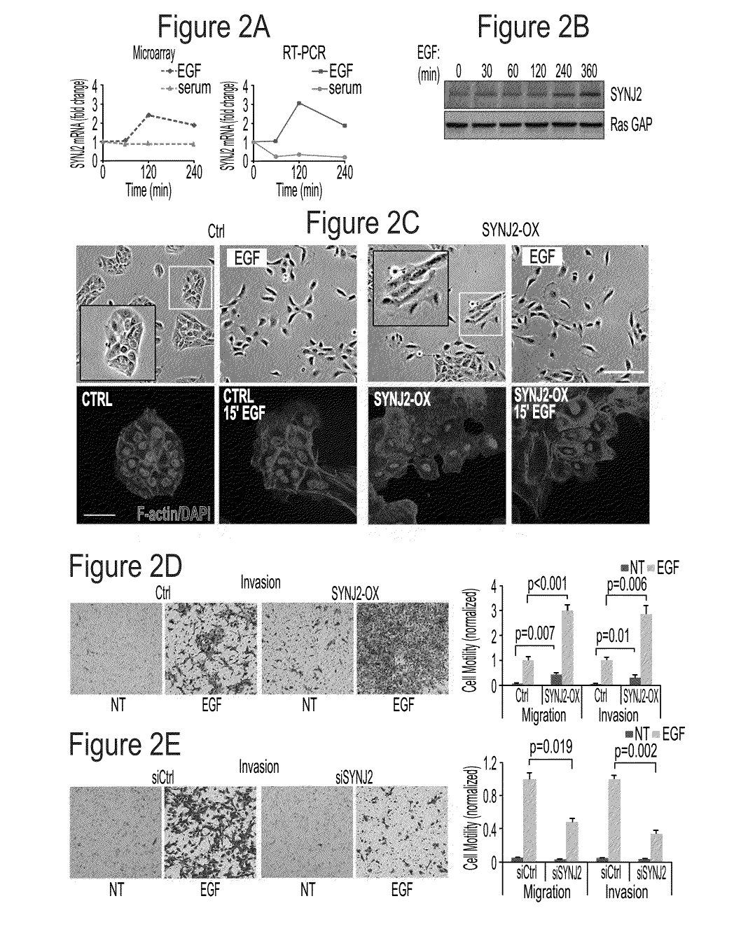

Human mammary epithelial cells (MCF10A) exhibit strong migratory and invasive phenotypes when cultured with EGF family ligands (FIGS. 1A and 1B), but treatment with serum is insufficient to propel cell motility. Co-incubation of EGF along with inhibitors of EGFR (AG1478), MEK (U0126) or PI3K (Wortmannin) reduced motility (FIG. 1C), suggesting that both MEK/ERK and PI3K activities are essential for EGF-induced migration Importantly, the EGFR-induced motile phenotype associates with transcriptional upregulation of 425 genes (Amit et al., 2007). To identify genes that propel metastasis, this gene-set was intersected with a larger set of genes that undergo up-regulation during in vivo selection of metastatic sub-clones of breast cancer cells (Minn et al., 2005). The group of 23 overlapping genes (FIG. 1D) included the gene encoding Synaptojanin-2 (SYNJ2), a lipid phosphatase implicated in glioma cell invasion (Chuang et al., 2004). EGF-induced up-regulation of SYNJ2 was validated by PCR and immunoblotting (FIGS. 2A and 2B).

Next, MCF10A cells were transformed and sub-cloned to stably overexpress SYNJ2 (as a GFP fusion; SYNJ2-OX, FIG. 1E). When plated in EGF-deprived medium, SYNJ2-OX cells displayed a pro-migratory phenotype characterized by membrane ruffling (FIG. 2C), along with enhanced basal and EGF-induced migratory and invasive capacities (FIGS. 2D and 2C). Conversely, knockdown of SYNJ2 using small interfering RNAs (siRNAs; FIG. 1G) significantly reduced cell invasion, as well as individual and collective migration (FIGS. 2E, 1H and 1J). In conclusion, EGF-induced up-regulation of SYNJ2 drives a robust invasive phenotype of mammary cells.

### Example 3

### The Phosphatase Activity of SYNJ2 is Essential for Invasiveness of Mammary Cells

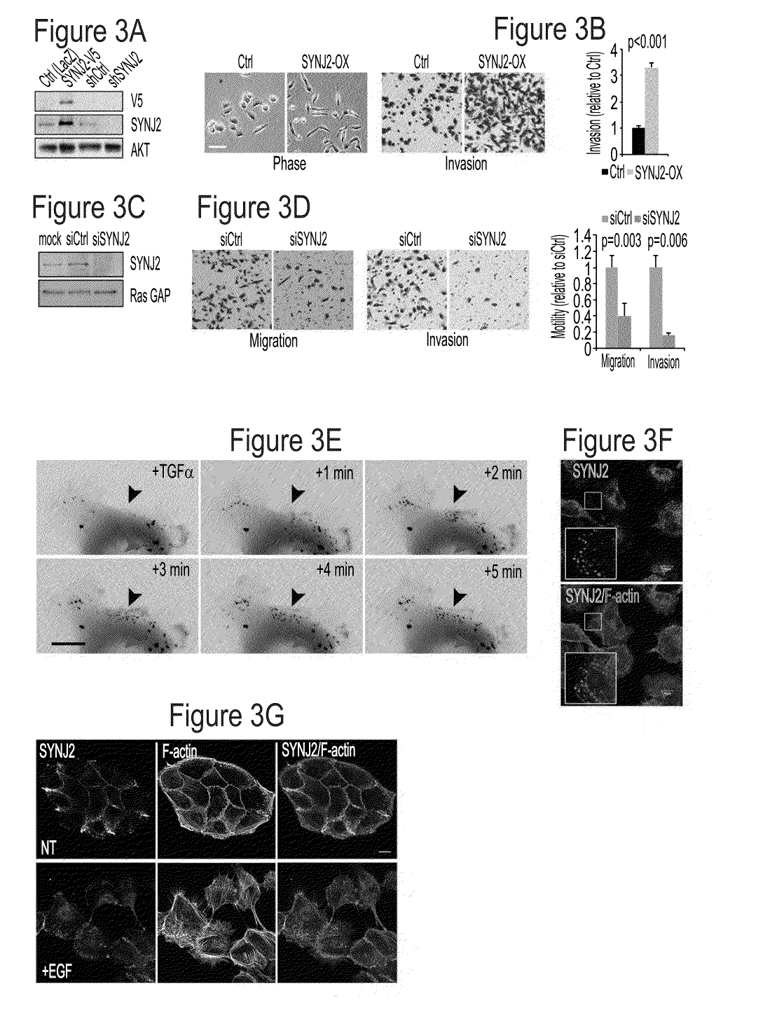

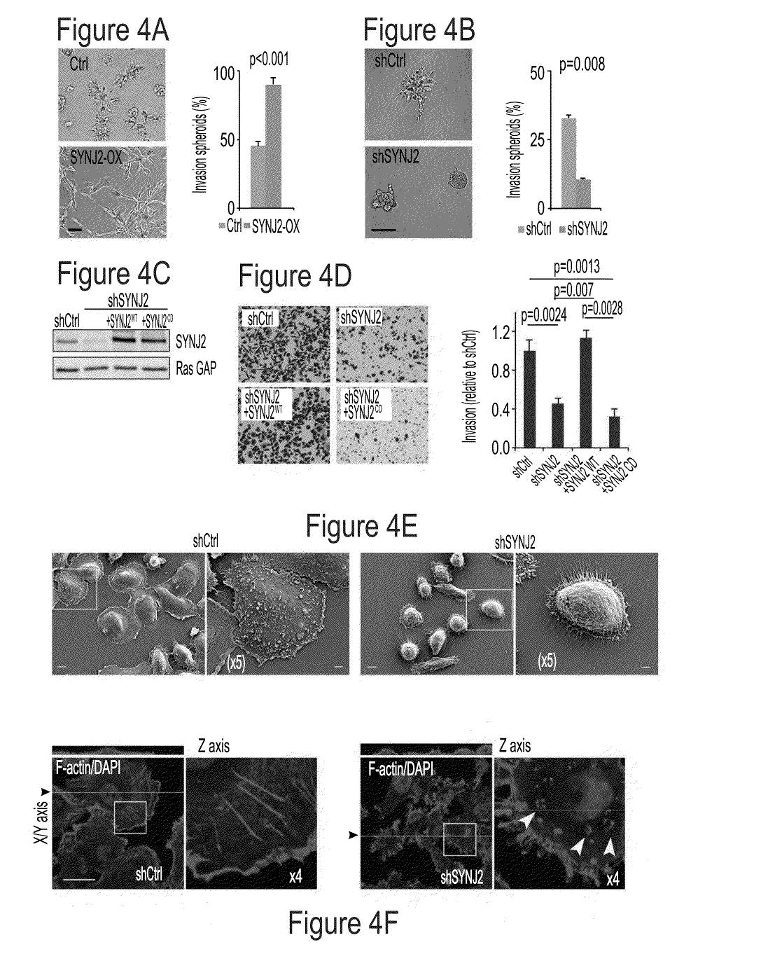

To enable in vivo experiments, the highly metastatic MDA-MB-231 breast cancer Red fluorescent protein (RFP) expressing cells were used to generate subclones overexpressing either SYNJ2 or LacZ (control), as well as sub-clones expressing shControl or SYNJ2-specific hairpins (shSYNJ2; FIG. 3A). Enhanced expression of SYNJ2 conferred an elongated morphology in 2D cultures (FIG. 3B) and extensive invasive arms, when cells were grown in 3D cultures (FIG. 4A). Conversely, SYNJ2 knockdown abrogated invasive patterns (FIG. 4B). Similarly, over-expression enhanced invasive capacities by ˜3.2 fold (FIG. 3B), and knockdown (FIG. 3C) inhibited migration and invasion (FIG. 3D). To examine roles for the catalytic phosphatase activity, shSYNJ2 cells with lentiviral particles encoding either a WT SYNJ2 or a catalytically-dead form (D388A and D726A; FIG. 4C) harboring point mutations in each of the conserved WXGDXN(F/Y)R motifs (Jefferson and Majerus, 1996) within the phosphatase/nuclease domain (Pfam: PF03372). Unlike WT SYNJ2, re-expression of the mutant failed to restore the invasive capacity (FIG. 4D), indicating that the phosphatase activity of SYNJ2 is essential for the invasive phenotype.

The failure of shSYNJ2 cells to migrate was further supported by both scanning electron microscopy (FIG. 4E) and F-actin staining, which revealed severe actin organization defects and an increase in cell height (FIG. 4F) Importantly, also noted were actin patches clustered around circular moieties (FIG. 4F; arrowheads). Accordingly, time-lapse microscopy analyses of shSYNJ2 cells confirmed the existence of abnormal intracellular vesicles, suggesting that SYNJ2 knockdown derailed vesicular trafficking. Next, the sub-cellular localization of SYNJ2 was examined Time-lapse images of MDA-MB-231 cells expressing GFP-SYNJ2 (FIG. 3E), as well as immunofluorescence using anti-SYNJ2 antibodies (FIG. 3F), reflected two major patterns of SYNJ2 distribution: small peripheral assemblies, which localized to the leading edge (black arrowheads in FIG. 3E), and a second population of larger assemblies, which were located closer to the cell centre (blue arrowheads). Notably, shortly after stimulation of MDA-MB-231 cells with an EGFR ligand (TGF-alpha), SYNJ2 rapidly assembled at the base of emerging lamellipodia, underneath the forming leading edge (FIGS. 3E, 3F). Interestingly, similar analyses performed with MCF10A cells indicated that SYNJ2 initially co-localizes with F-actin at cell-to-cell junctions, but translocates to the leading edge, typically to the base of lamellipodia, upon stimulation with EGF (FIG. 3G). In conclusion, these observations indicate that growth factors regulate not only the levels of SYNJ2 expression, but also its dynamic recruitment to the leading edge.

### Example 4

### Recruitment of SYNJ2 to the Ventral Membrane Depends on Dynamin and Rac1

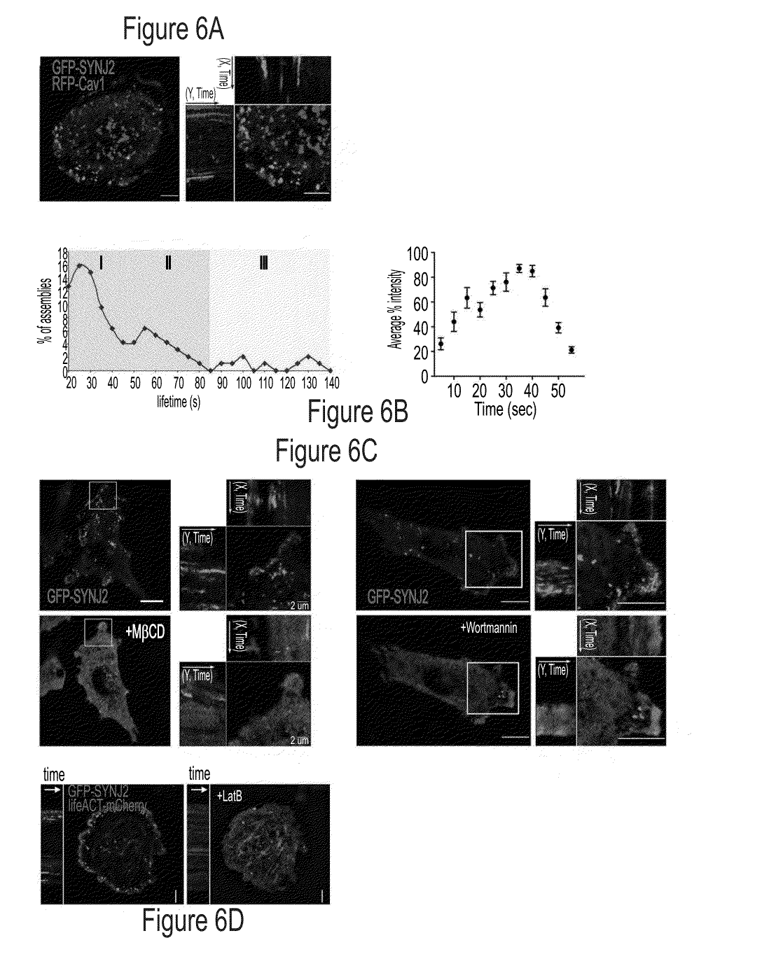

To investigate the dynamics of SYNJ2's sites of localization, a stably expressing GFP-SYNJ2 MDA-MB-231 subclone (GFP-SYNJ2 cells) was generated and analyzed for the formation and consumption of GFP-SYNJ2 puncta. These were classified into kinetically distinct sub-populations: dynamic puncta that localized to ruffling membranes and puncta localized to discrete regions proximal to the cell centre (FIG. 5A). Notably, GFP-SYNJ2 puncta showed minimal overlap with assemblies marked by RFP-Clathrin light chain A (FIG. 5A) or RFP-Caveolin 1 (FIG. 6A), suggesting minor localization to Clathrin-coated pits or to caveolae Importantly, newly formed peripheral puncta heralded nascent lamellipodia, as their appearance preceded local formation of lamellipodia. In contrast, the more central and stable clusters of puncta, which co-localized with actin, persisted for ˜30 minutes (FIG. 5B). Accordingly, tracking of individual assemblies (FIG. 6B; left) revealed remarkably wide distribution of lifetimes: short-lived (˜20-40 s, 60% of assemblies), intermediate lifetimes, and long-lived assemblies (˜10% of assemblies). Initiation of the intermediate group was followed by a continuous increase in fluorescence intensity, while the assembly remained static in terms of movement (FIG. 6B; right). This dynamic pattern resembles that of Clathrin-coated pits (Ehrlich et al., 2004) and suggests the formation and consumption of trafficking intermediates.

The mostly bimodal compartmentalization of GFP-SYNJ2 at the ventral membrane was reinforced by the synchronous appearance and disappearance of fluorescence signals in experiments employing both epifluorescence (red; relatively insensitive to changes in the Z dimension) and total internal reflection microscopy (TIRF, green; limited to ˜200 nm depth). Because puncta appeared yellow throughout their lifetime (FIG. 5C), the present inventors concluded that SYNJ2 assembles within the plane of the ventral plasma membrane. By employing a panel of inhibitors it was found that the assembly was dramatically inhibited by cholesterol depletion (FIG. 6C; left), suggesting that cholesterol-rich membrane microdomains are needed for SYNJ2 recruitment to the ventral membrane. A similar inhibitory effect was induced by Wortmannin (FIG. 6C; right), suggesting a role for PI3K. Another requirement was revealed by employing Dyngo-4a, an inhibitor of Dynamin, the large GTPase that mediates the scission step of clathrin-dependent and clathrin-independent carriers, and whose inhibition leads to accumulation of U-shape invagination intermediates (Macia et al., 2006). Because Dyngo-4a strongly arrested the dynamic assemblies of SYNJ2 at the plasma membrane (FIG. 5D), the present inventors concluded that SYNJ2 is recruited to nascent trafficking intermediates regulated by Dynamin. Because Dynamin has been implicated as a facilitator of cell migration and invasion (Kruchten and McNiven, 2006), its physical interactions with SYNJ2 was tested. This experiment confirmed complex formation between active Dynamin and SYNJ2 (FIG. 5E), in line with an extended role for Dynamin in both endocytosis and actin-based migration.

SYNJ2 can physically interact with GTP-loaded Rac1 (Malecz et al., 2000), and inducible activation of Rac1 requires internalization and subsequent recycling (Palamidessi et al., 2008). Hence, the coincidence of the peripheral puncta of SYNJ2 coincide with Rac1 was tested. Indeed, immunostaining of endogenous Rac1 revealed co-localization with peripheral puncta of GFP-SYNJ2 (FIG. 5F). Moreover, inhibition of GTP loading onto Rac1 (using NSC-23766) dramatically reduced the number of GFP-SYNJ2 puncta (FIG. 5G). Complementarily, SYNJ2 knockdown reduced the levels of GTP-loaded Rac1 in MDA-MB-231 cells (FIG. 5H). In accord with a regulatory role for Rac1 and the actin cytoskeleton in recruiting SYNJ2 to the membrane, inhibition of actin dynamics with Latrunculin abrogated GFP-SYNJ2 dynamics (FIG. 6D). Taken together, these results associate the peripheral SYNJ2 assemblies, with a dynamin-mediated endocytic pathway that depends on cholesterol, 3′-phosphoinositides, actin and active Rac1. Notably, this pathway shares several attributes with clathrin-independent carriers that enable rapid membrane and adhesion turnover at the leading edge of migrating fibroblasts (Howes et al., 2010).

### Example 5

### SYNJ2 Controls Vesicular Trafficking of Cell Surface Receptors

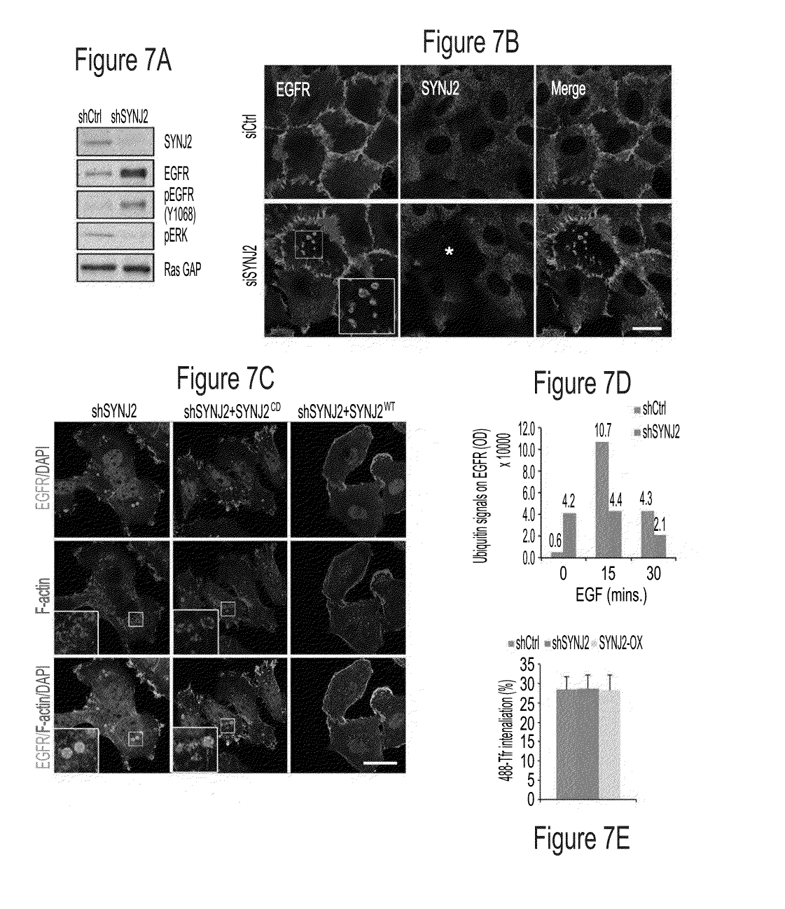

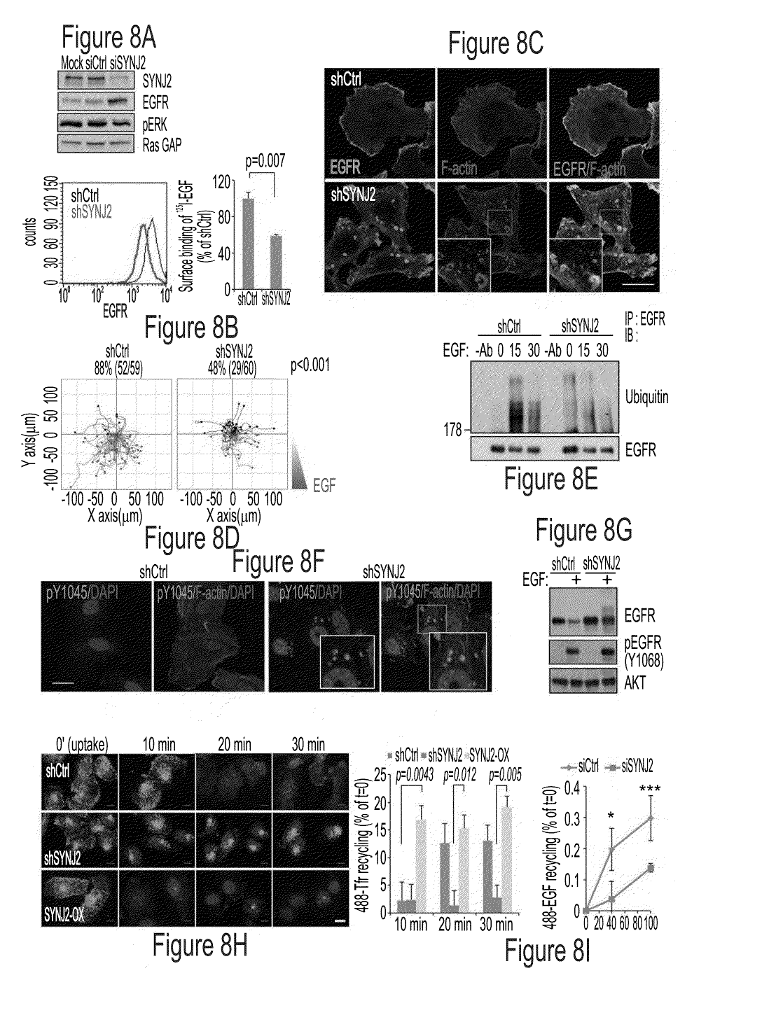

Although EGF-treated shSYNJ2-MCF10A cells displayed higher levels of total and phosphorylated EGFR relative to control cells, this translated to lower, rather than higher activation of ERK (FIG. 7A). Along this line, it was noted that SYNJ2 knockdown trapped EGFRs in enlarged intracellular vesicles (FIG. 7B). Consistent with trapping, immunoblotting of MDA-MB-231 cells similarly revealed that EGFR levels were stabilized in siSYNJ2 cells (FIG. 8A), but quantification of surface EGFR by using two methods indicated significantly lower surface levels (FIG. 8B). Intracellular trapping of EGFR bears functional consequences: in line with their well-characterized chemotactic function (Mouneimne et al., 2006; van Rheenen et al., 2007), EGFRs localized to the leading edge of mammary cells, but EGFRs of shSYNJ2 cells lost their polarized distribution and accumulated in large, actin-decorated vesicles (FIG. 8C). Notably, EGFR trafficking defects observed in shSYNJ2 cells could be rescued by WT SYNJ2, but not by a catalytically-dead form (FIG. 7C), indicating that the phosphatase activity of SYNJ2 is essential for vesicular trafficking of EGFRs to and from the leading edge, where it mediates the chemotactic response to gradients of EGF. Consistent with this model, shSYNJ2 cells severely lost the ability to migrate along a gradient of EGF (FIG. 8D).

The abnormal accumulation of EGFR in SYNJ2-depleted cells could reflect defects in EGFR delivery, arrested recycling, or impaired sorting for degradation, a process regulated by ubiquitination (Goh et al., 2010). Consistent with impaired sorting, SYNJ2-depleted cells exhibited significantly higher basal EGFR ubiquitination, which was only weakly altered in response to EGF (FIGS. 8E and 7D). Furthermore, despite being tagged for degradation by phosphorylation of tyrosine 1045 (a docking site for the ubiquitin ligase c-Cbl; FIG. 8F), an EGF stimulation experiment confirmed normal activation (tyrosine 1068 phosphorylation) but defective degradation in shSYNJ2 cells (FIG. 8G). To address a recycling defect, fluorescent ligands were employed to follow the extensive recycling of the transferrin receptor (TfR), as well as the weaker recycling of EGFR. Although TfR internalization was not affected, recycling was markedly decreased in shSYNJ2 cells and, conversely, markedly accelerated in SYNJ2-OX cells (FIGS. 8H and 7E). Likewise, flow cytometry analyses indicated defective recycling of fluorescent-EGF (FIG. 8I), and live cell imaging confirmed ligand accumulation within the large vesicles of SYNJ2-depleted cells. In conclusion, these results indicate that SYNJ2 is essential for proper recycling of both EGFR and TfR.

### Example 6

### SYNJ2 Knockdown Perturbs Homeostasis of Phosphoinositol Lipids and Alters Both Endocytosis and Adhesion

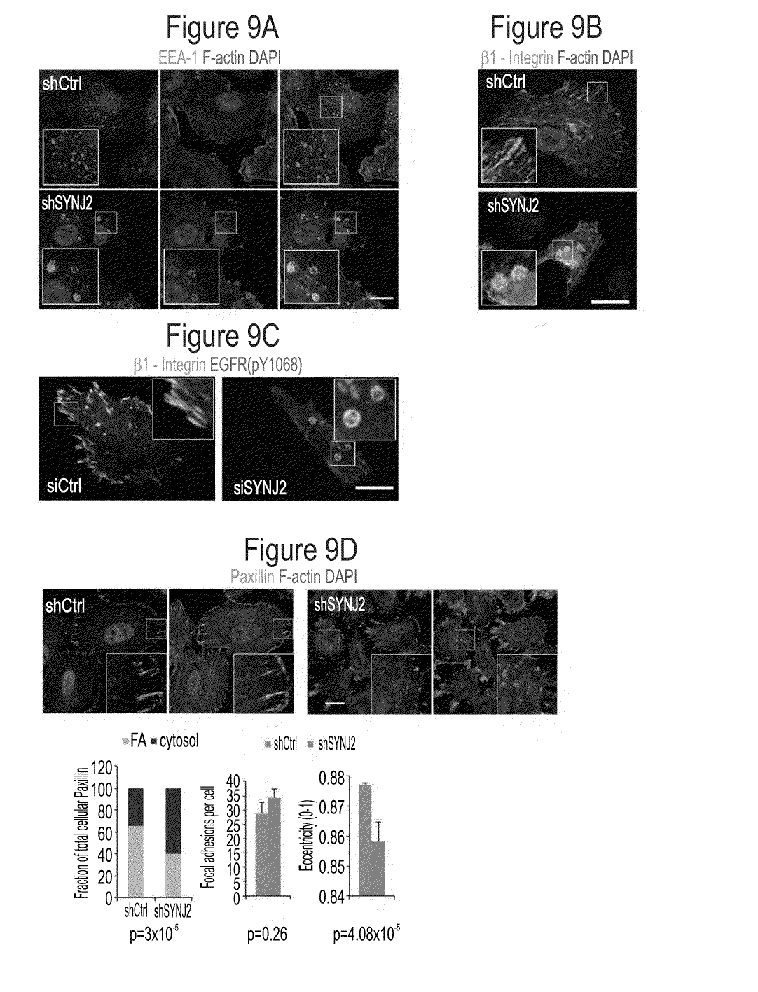

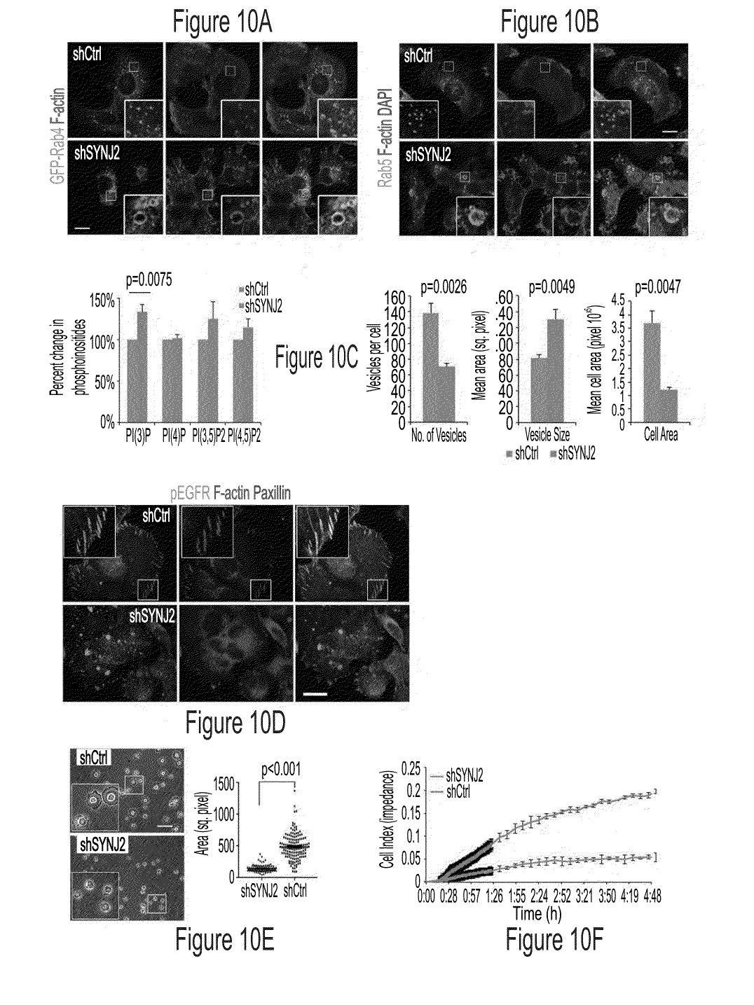

The endocytic system maintains several distinct compartments, which are defined by specific phosphoinositides (PI) (Gruenberg and Stenmark, 2004), and the present analyses uncovered strong dependency on SYNJ2. For example, by probing early endosomes for EEA1, a PI(3)P-binder, it was found that its spatial organization was markedly altered in SYNJ2-depleted cells (FIG. 9A). Similarly, probing the recycling compartment using GFP-tagged Rab4, uncovered strong associations with the circular actin patches of shSYNJ2 cells (FIG. 10A). The distribution of another marker of early endosome, Rab5, also reflected dependence on SYNJ2 (FIG. 10B). Whereas the number of Rab5-positive vesicles was significantly lower in shSYNJ2-depleted cells, their average size increased and they partly localized to circular actin patches (FIG. 9A). To uncover underlying alterations in phosphoinositides, shCtrl and shSYNJ2 MDA-MB-231 cells that were biosynthetically labeled were compared, and thereafter their phospholipids were extracted (FIG. 10C). The results showed that mainly PI(3)P, but also PI(4,5)P₂ and PI(3,5)P₂ were present at higher levels in shSYNJ2 cells, whereas PI(4)P levels remained unaltered and levels of both PI(3,4)P₂ and PI(3,4,5)P₃ were hardly detectable by this method. While these results confirm the notion that SYNJ2 targets primarily the D5 position of PIs, the present inventors assume that the rather limited global effects observed represent larger local differences. In conclusion, these observations reaffirm that SYNJ2 controls cargo sorting at the early endosome, as well as in the subsequent recycling step.

Along with recycling of RTKs like EGFR, vesicular trafficking of integrins and their interactions with downstream partners, such as Paxillin, play major roles in cell migration and focal adhesion (FA) maturation (Guo and Giancotti, 2004). Accordingly, beta-1 integrin and phosphorylated-EGFR (pEGFR) localized to FAs of MDA-MB-231 cells. By contrast, due to abnormal accumulation in large vesicles, both proteins failed to localize to the periphery of SYNJ2-depleted cells (FIG. 10D, S5B and S5C). Moreover, using Paxillin as a marker of mature FAs, it was found that FAs assumed a round and relatively short appearance in shSYNJ2 cells (FIG. 9D). Taken together, these observations imply that SYNJ2 is required for substrate adhesion, a scenario examined by measuring cell spreading using two methods (FIGS. 10E and 10F). The results demonstrated attenuated adhesion of shSYNJ2 cells, which is attributed to defective delivery of both integrins and RTKs to FAs.

### Example 7

### SYNJ2 Regulates the Assembly of Invadopodia

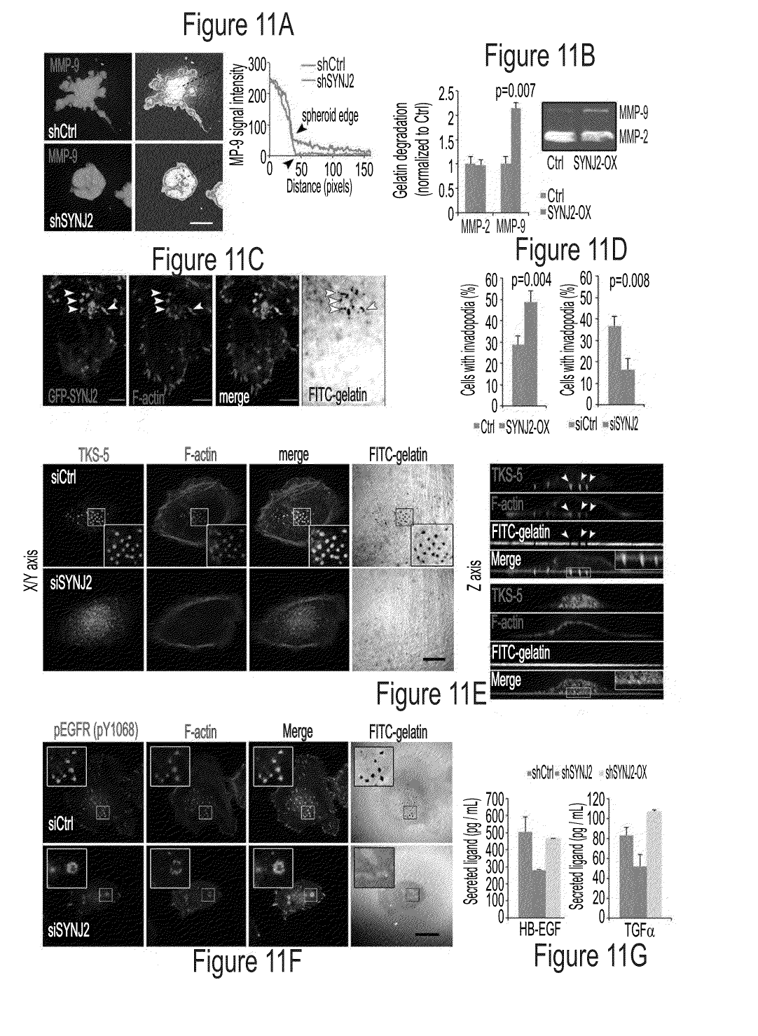

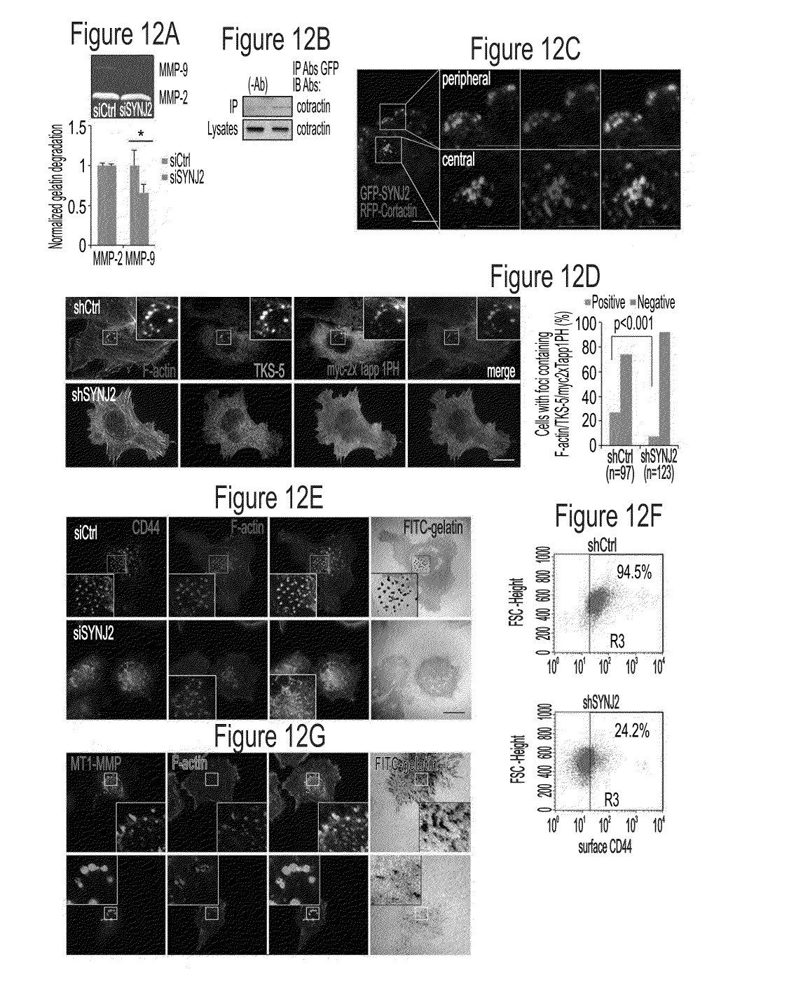

Matrix-based 3D cultures of MDA-MB-231 cells normally display wedge-shaped protrusions, but shSYNJ2 cells displayed roundish extensions (FIG. 11A), suggesting defective matrix degradation. To test this, confocal immunofluorescence images of MMP-9 were obtained, and it was noted that shSYNJ2 spheroids displayed a relatively sharp decrease of MMP-9 abundance at their borders (FIG. 11A), likely due to impaired secretion. Indeed, zymography assays performed on conditioned media confirmed defective MMP-9 secretion by cells that were treated with siSYNJ2 oligonucleotides, but MMP-2 secretion remained unaltered (FIG. 12A). Conversely, media conditioned by cells overexpressing SYNJ2 displayed a substantial increase in MMP-9 activity (FIG. 11B), in line involvement of SYNJ2 in MMP secretion.

To visualize focal proteolysis, cells were plated on cross-linked fluorescent gelatin and probed for the actin-centered, matrix-degrading organelles called invadopodia (Murphy and Courtneidge, 2011). In line with previous reports, active matrix proteolysis corresponded to actin dots localized underneath the cell body. Importantly, SYNJ2-GFP puncta co-localized with these structures (FIG. 11C, arrowheads), which resembled the actin-associated long-lived puncta presented in FIG. 5B. Expression levels of SYNJ2 are clearly correlated with invadopodia occurrence; whereas SYNJ2 overexpression almost doubled the fraction of invadopodia-containing cells, siSYNJ2 significantly reduced the incidence of invadopodia (FIG. 11D), implying causal relationships. Next, potential physical associations between SYNJ2 and Cortactin, a well-characterized marker of invadopodia, was examined and found that SYNJ2 and Cortactin co-immunoprecipitate (FIG. 12B), as well as co-localize to both invadopodia and leading edges (FIG. 12C). To firmly establish a driving role for SYNJ2, TKS5 was observed, a PI(3,4)P₂ and a binder of Cortactin that serves as a signpost of invadopodia (Courtneidge et al., 2005). As expected, endogenous TKS5 localized to multiple ventral sites of matrix degradation in control MDA-MB-231 cells, but almost no active sites were found in siSYNJ2 cells, and TKS5 lost its ventral location (FIG. 6E; X-Y and Z panels). Furthermore, because invadopodial TKS5 anchors at PI(3,4)P₂ (Oikawa et al., 2008), a PI(3,4)P₂-binding domain, namely the PH domain of Tapp1 was used as a probe. Consistent with previous reports, ectopic expression of the PH domain reduced the number of invadopodia, but nevertheless the remaining signal co-localized with TKS5 and actin cores (FIG. 12D). In conclusion, SYNJ2 appears necessary at a step preceding TKS5 engagement, consistent with sequential action of PI3K (Yamaguchi et al., 2011) and SYNJ2, which respectively generate PI(3,4,5)P₃ and then PI(3,4)P₂, to anchor TKS5 at sites of EGFR-induced activation of PI3K.

In line with an EGFR-PI3K-SYNJ2 scenario, the active form of EGFR (pEGFR) was detected in proteolytically active invadopodia, but EGFRs of SYNJ2-depleted cells localized to swollen vesicles (FIG. 11F). The mechanism responsible for local receptor activation remains unknown. According to one model, cleavage of pro-ligands, such as the heparin-binding EGF (HB-EGF), by a complex comprising MMP-7 and CD44, might locally stimulate EGFR (Yu et al., 2002). In line with this model, SYNJ2 abundance was correlated with secretion of EGFR ligands (FIG. 11G), and detected co-localization of CD44 with the actin cores of invadopodia (FIG. 12E). Likewise, using flow cytometry, it was found that surface expression of CD44 was strongly suppressed in shSYNJ2 cells relative to control cells (FIG. 12F). Yet another critical step in the maturation of invadopodia is the recruitment of the membrane type-1 matrix metalloproteinase (MT1-MMP), which activates soluble MMPs (Wang and McNiven, 2012). Accordingly, it was found that in control cells MT1-MMP corresponded to sites of invadopodial protrusions, but MT1-MMP molecules of siSYNJ2 cells formed large aggregates, which were not associated with matrix degradation (FIG. 9E). Taken together, these observations imply that SYNJ2 is essential for invadopodia priming, as well as for targeting to this organelle both proteases and two previously unrecognized residents, CD44 and an active EGFR.

### Example 8

### SYNJ2 Promotes Tumor Growth and Metastatic Spread in a Mammary Animal Model

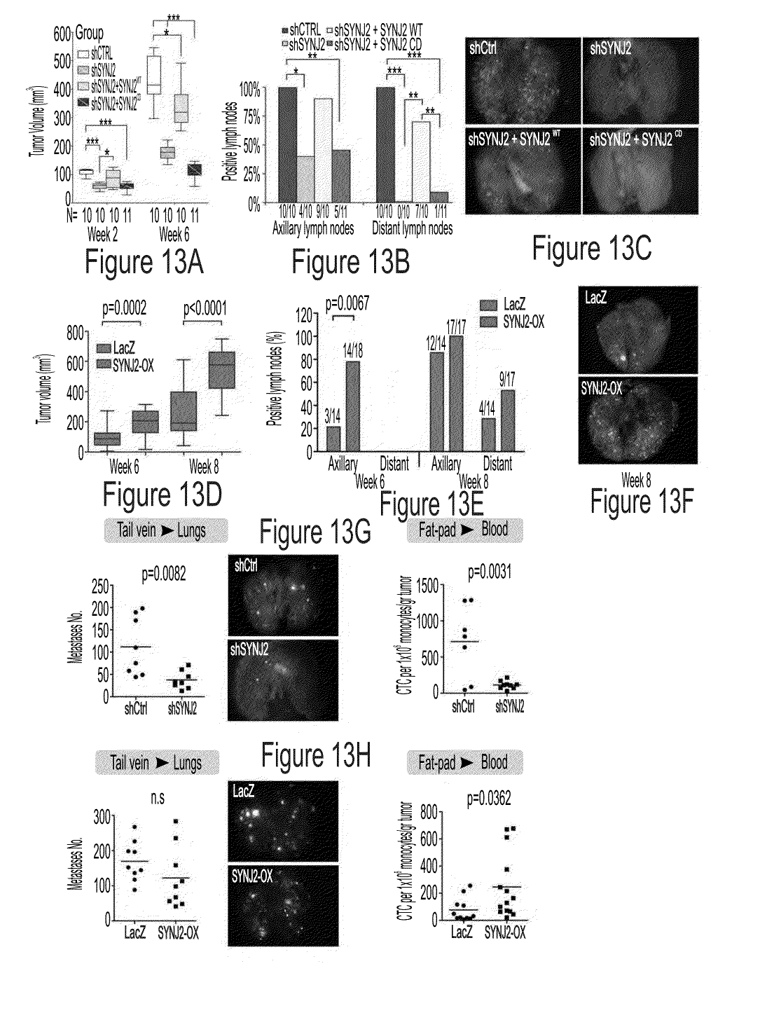

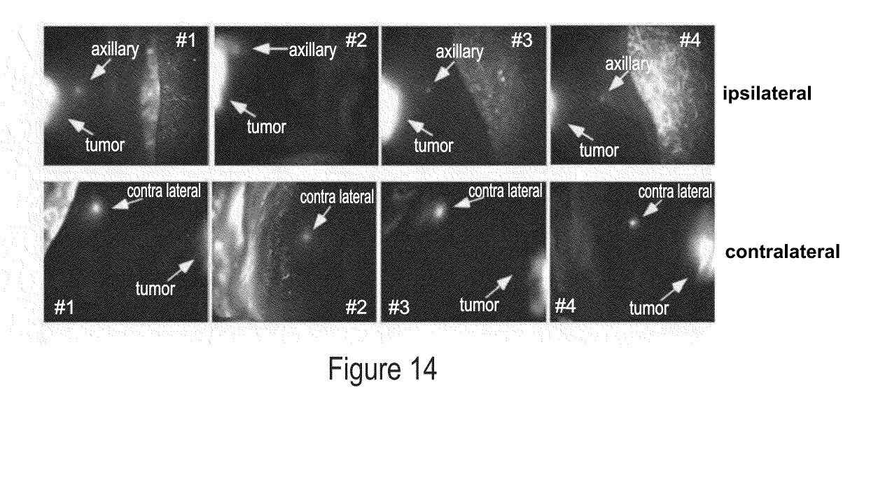

To assess the effect of SYNJ2 on metastatic dissemination in vivo MDA-MB-231-RFP cells (and derivatives) were implanted into the mammary fat pad of female mice, and two or six weeks later measured both tumor size (FIG. 13A) and metastases (FIG. 13B). Primary tumor growth was significantly faster in the shCtrl and shSYNJ2+SYNJ2^{WT} (‘active rescue’) groups, relative to the shSYNJ2 and the ‘inactive rescue’ (shSYNJ2+SYNJ2^{CD}) groups. The metastatic behavior similarly correlated with SYNJ2: the shSYNJ2 and the ‘inactive rescue’ group displayed significant reduction in metastatic spread to local and distant lymph nodes (FIGS. 13B and 14). In order to examine distant metastases, mice were sacrificed and their lungs evaluated. Lungs of animals implanted with shSYNJ2 cells, or the ‘inactive rescue’ cells, showed a dramatic reduction in the number and size of metastases, compared to animals inoculated with the shCtrl or the ‘active rescue’ cells (FIG. 13C). Taken together, these results implicate SYNJ2 in metastasis promotion.

Similarly, xenografts overexpressing SYNJ2 were monitored. As expected, SYNJ2-OX cells gave rise to faster growing tumors (FIG. 13D), and they also displayed earlier onset of nodal metastases (FIG. 13E). Consistent with robust lymphatic invasion, the lungs of animals implanted with SYNJ2-OX cells showed an increase in the number of metastases (FIG. 13F). Next, the effect of SYNJ2 on intravasation or extravasation was tested. Hence, sub-clones of MDA-MB-231-RFP cells were either directly injected into the circulation (tail vein) of female mice and scored for lung colonization (extravasation), or they were implanted in the fat pad and scored in blood as circulating tumor cells (CTCs; intravasation). Note that these experiments took into account the size differences between the respective primary tumors. The normalized results indicated that SYNJ2 is necessary for both intravasation (p=0.0031) and extravasation (p=0.0082; FIG. 13G). This conclusion was further tested by using GFP-SYNJ2 overexpressing cells (FIG. 13H). Notably, the intravasation results obtained in this experiment displayed statistical significance, but the ability of SYNJ2-OX cells to better extravasate and colonize a distant organ did not reach significance, suggesting that the observed strong effects of SYNJ2 on local and distant metastasis are primarily due to enhanced intravasation into lymph and blood vessels.

### Example 9

### SYNJ2 is Associated with Aggressive Human Breast Tumors

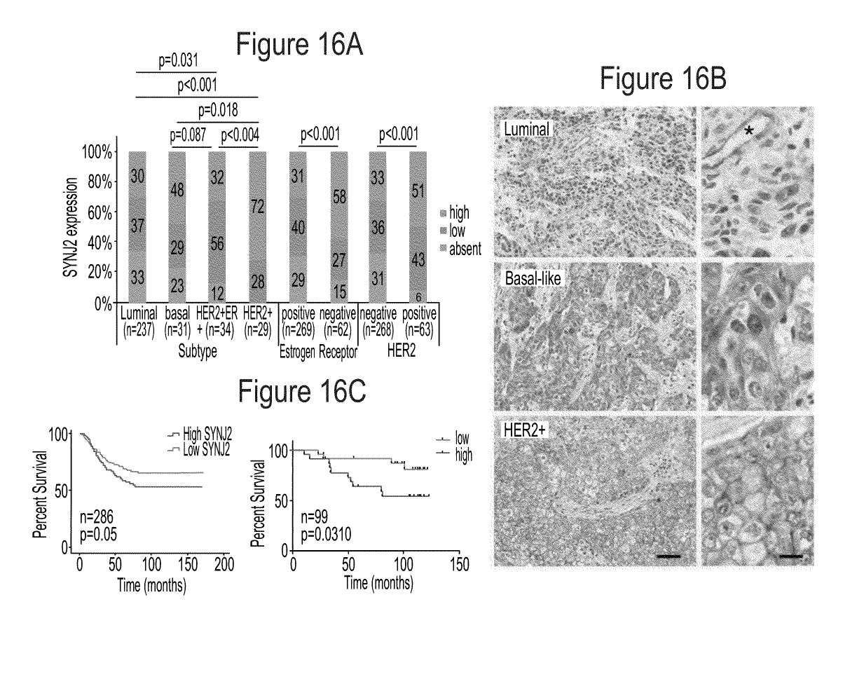

To address SYNJ2's relevance to human cancer, the transcript levels of SYNJ2 were analyzed in the NCI-60 panel of 60 human cancer lines. In line with contribution to motile phenotypes, it was found that high transcript levels of SYNJ2 associate with mesenchymal phenotypes. Next, a set of 331 paraffin-embedded samples of breast carcinomas NJ2 were immunostained (FIG. 16A) Importantly, expression intensity of SYNJ2 was positively associated with prognostically unfavorable subtypes defined by HER2 overexpression (p<0.001) and/or lack of estrogen receptor (p<0.001). However, no significant association was found between SYNJ2 abundance and age, histological subtype, axillary lymph node status, and differentiation grade. Interestingly, staining patterns for SYNJ2 also varied; whereas HER2+ tumors exhibited mostly membranal staining, luminal and triple negative tumors displayed cytoplasmic staining (FIG. 16B). To support the findings, SYNJ2 mRNA levels were analyzed in two cohorts of breast cancer specimens and an association was found with shorter patient survival rates (FIG. 16C). Altogether, these observations support involvement of SYNJ2 in progression of breast cancer, but they leave open the mechanism behind transcript up-regulation.

In summary, the observations made in animals, along with the clinical data and the in vitro experiments, clearly indicate that dephosphorylation of inositol lipids by SYNJ2 is critical for the metastatic process, primarily because of the cardinal roles played by phosphoinositides in trafficking of cell surface molecules to and from invadopodia and the leading edge. Below is presented a working model (FIG. 15) and discuss the multiple functions of SYNJ2 in the broad context of tumor progression.

### Example 10

### Selective Inhibitors of the 5′ Phosphatase Activity of SYNJ2

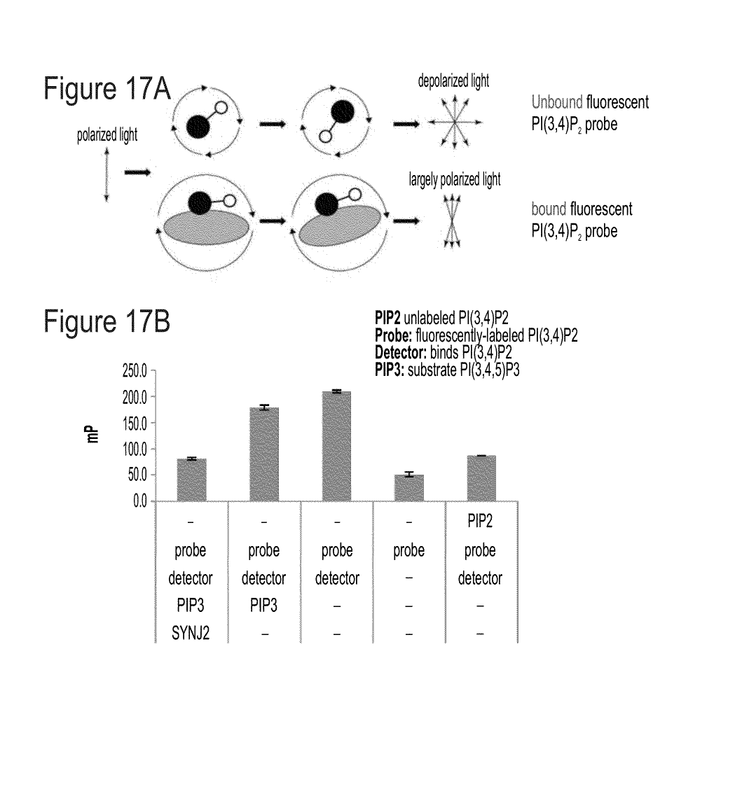

In order to identify selective inhibitors of SYNJ2 phosphatase activity, the present inventors utilized a fluorescence polarization competitive assay relying on the principle that molecules are constantly rotating and moving in space but once bound to another bigger element (e.g., a protein) their movement is dramatically limited. These changes in movement can be detected and measured using fluorescent molecules (i.e., probes) that in unbound state give rise to very low polarization readings, but when a detector (e.g., a binding protein) that binds these molecules is added to the solution, the fluorescent molecules are stabilized in a confined composition that increases the polarization readings in the solution (see FIG. 17A).

In the screen preformed, the present inventors measured the enzymatic activity of SYNJ2 to de-phosphorylate the 5′ position of PI(3,4,5,)P3 to produce PI(3,4)P2, in the presence of different compounds. Once the enzymatic reaction was completed/stopped, the solution containing the PI(3,4)P2 products was mixed with a mixture of PI(3,4)P2 binding protein (detector) and a fluorescent PI(3,4)P2 (probe). The detector protein used was the purified PH-domain of Tapp1 that selectively binds PI(3,4)P2 (SEQ ID NO.: 15). As demonstrated in FIG. 17B, the polarization values measured in this assay decreased as the bound PI(3,4)P2 fluorescent probes were being displaced by un-labeled PI(3,4)P2 produced by the enzymatic activity of SYNJ2 and the amount of unbound fluorescent probe in the solution increased.

Table 2 below depicts the various compounds identified using this method that were able to inhibit the production of PI(3,4)P2 by SYNJ2.

TABLE 2
Identified selective inhibitors of SYNJ2

| | AnalytiCon | | | |
|---|---|---|---|---|
| | Cat. No. | | IC50 | |
| No | [CAS No.] | Compound name | (μM) | Structure |
| 1 | NP-001872 | Ampelopsin | 3.17 | 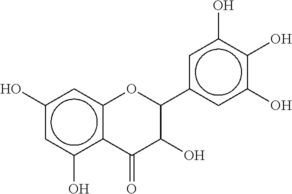 |
| 2 | NP-003195 | 3-Hydroxy-5- phenylpentanoic acid 3-D- glucopyranoside | 1.87 | 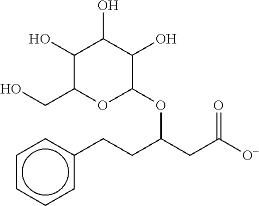 |
| 3 | NP-012228 | Cedeodarin | 2.19 | 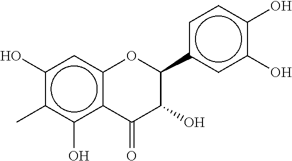 |
| 4 | NP-014109 | (−)-Catechin-7- gallate | 3.44 | 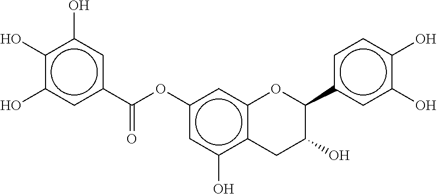 |
| 5 | NP-015304 | Paeonivayin | 6.02 | 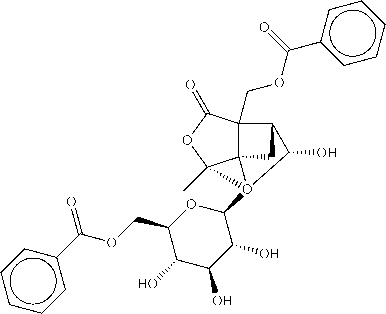 |
| 6 | NP-003491 | Sericoside | 3.52 | 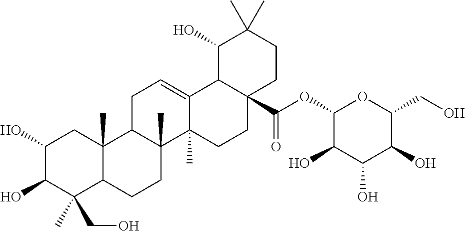 |
| 7 | NP-000303 | Keioside | 3.4 | 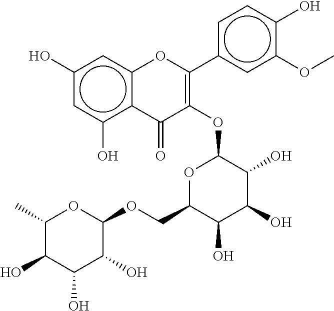 |
| 8 | NP-005201 | 2,4,3′,4′- tetrahydroxy-6- methoxy- benzophenone | 2.84 | 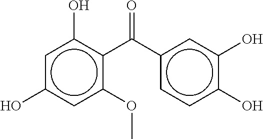 |
| 9 | NP-001585 | 2-Hydroxy-1,8- cineole- glucopyranoside | 1.98 | 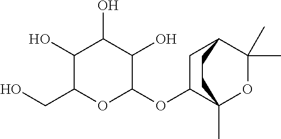 [SMILES: CC1(C)O[C@@]2(C)CC[C@@H]1CC2OC1OC(CO)C(O)C(O)C1O] |
| 10 | NP-014110 | (−)-Gallocatechin- 7-gallate | 1.075 | 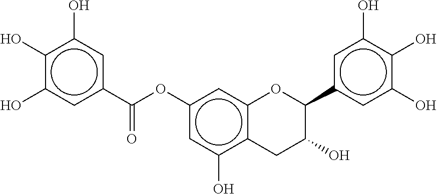 |
| 11 | NP-002326 | (−)-Epicatechin-3- gallate | 2.88 | 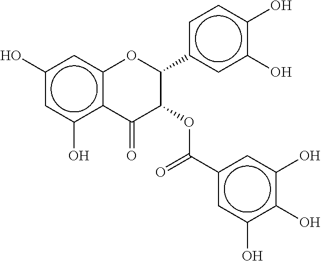 |
| 12 | | NP-000360 | 1.67 | 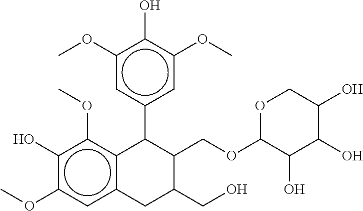 |
| 13 | NP-002973 | 1,6- Digalloylglucose | 7.53 | 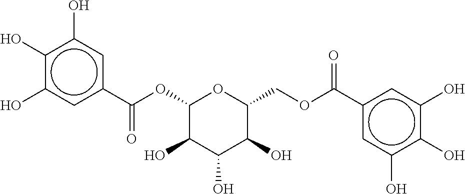 |
| 14 | NP-015222 | 3,7-dimethyloct- 2-ene-1,4,7-triol 1-β-D- glucopyranoside | 1.24 | 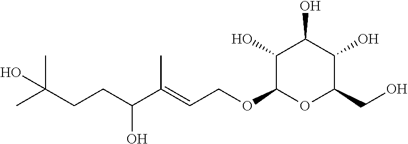 [SMILES: C/C(=C\CO[C@@H]1O[C@H](CO)[C@@H](O)[C@H](O)[C@H]1O)C(O)CCC(C)(C)O] |
| 15 | NP-000181 | Pyracanthoside | 0.874 | 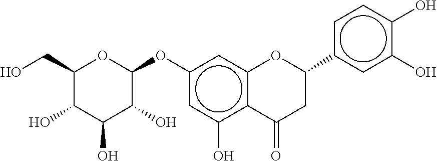 |
| 16 | NP-008708 | Isobiflorin | 2.71 | 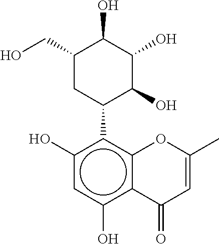 |
| 17 | [CAS: 55-56-1] | Chlorhexidine | 10 | 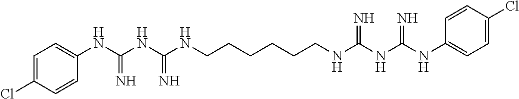 [SMILES: N=C(NCCCCCCNC(=N)NC(=N)Nc1ccc(Cl)cc1)NC(=N)Nc1ccc(Cl)cc1] |
| 18 | [CAS: 3546-41-6] | Pyrvinium pamoate | 1.25 | 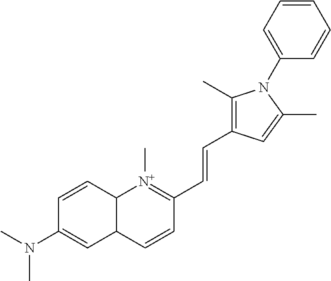 [SMILES: Cc1cc(/C=C/C2=[N+](C)C3C=CC(N(C)C)=CC3C=C2)c(C)n1-c1ccccc1] |
| 19 | NP-003299 | Quercitrin gallate | 2.94 | 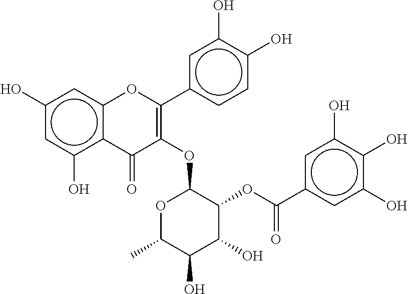 |
| 20 | NP-013254 | 4′-O-Methyl-3,5- di-O- caffeoylquinic acid | 3.17 | 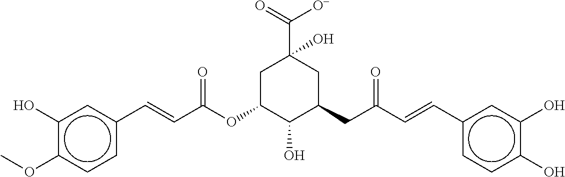 |
| 21 | NP-012429 | 3,5-Di-O- galloylshikimic acid | 3.75 | 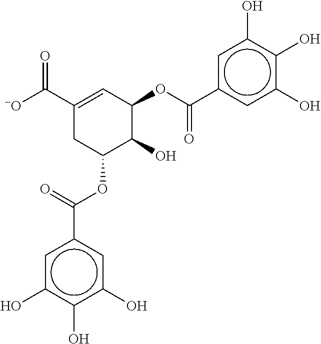 |
| 22 | NP-002325 | Epiafzelechin | 15.9 | 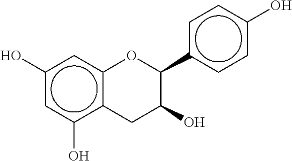 |
| 23 | NP-003143 | Oxyresveratrol | 6.65 | 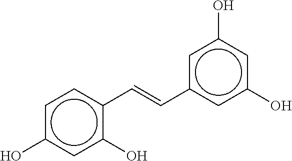 |
| 24 | NP-012649 | 6,8-di-(2,3- dihydroxybenzyl)- pinocembrin | 10.3 | 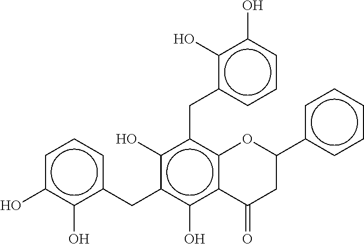 |

### Discussion

The functions of SYNJ2 as an integrative master regulator of cell migration and tumor metastasis are demonstrated, likely due to its ability to control the levels of PI phospholipids acting as both second messengers and signposts determining the identity of specific membrane sub-domains. Another reflection of the multiplicity of SYNJ2's action is the mostly bimodal ventral localization to invadopodia and lamellipodia. Accordingly, SYNJ2 forms physical complexes with prominent regulators of actin dynamics (e.g., Dynamin, Cortactin and Rac1). One key to understanding SYNJ2's action is the ability to control endocytic trafficking. Frequent packaging of portions of the plasma membrane in vesicles, which constantly feed actin-based protrusions propels cell migration (Ridley, 2011). SYNJ2 of lamellipodia exhibits remarkable dynamism (FIG. 3B), and live-cell imaging implies that SYNJ2 recruitment marks sites of new lamellipodia formation. Because SYNJ2 molecules located at the leading edge depend on Dynamin, Rac1, actin polymerization and cholesterol, but its distribution is distinct from that of Caveolin-1 and Clathrin, the present inventors assume that it represents a Dynamin-dependent variant of the Clathrin-independent carriers (CLICs), which sustain membrane turnover at the leading edge (Howes et al., 2010).

A series of elegant studies implicated SYNJ1 in synaptic vesicle recycling in neurons (Cremona et al., 1999). In mice, deletion of SYNJ1 caused elevation of steady-state PI(4,5)P₂, accumulation of Clathrin-coated vesicles, and a delay in post-endocytic vesicle reavailability (Mani et al., 2007). These observations suggest that dephosphorylation of PI(4,5)P₂, which would allow vesicle coat shedding, underlies the phenotype. In analogy, the present SYNJ2-depleted mammary cells displayed intracellular accumulation of active EGFRs. Receptor ubiquitination status and markers of the endocytic pathway indicated that trafficking is arrested at sorting endosomes, where internalized receptors are normally shunted to either recycling or degradation. Conceivably, the defect is due to an inability to disassemble PI(4,5)P₂-binding proteins associated with the vesicle's coat or with its actin comet tails (Kaksonen et al., 2003). Thus, in similarity to the defects in synaptic transmission observed upon SYNJ1 ablation, loss of SYNJ2 severely impairs cell migration and invasion due to arrested trafficking of surface molecules essential for motility.

The observed elevation of PI(3)P and PI(3,5)P₂ (FIG. 5C), regulators of early and late endosomes, respectively, in SYNJ2-depleted cells proposes additional trafficking mechanisms. A regulatory role for PI(3,5)P₂ has been reinforced by the identification of multiple binders, such as integrins and several Rab proteins (Catimel et al., 2008). PI(3)P is phosphorylated by PIKfyve, a 5-kinase implicated in cycling between endosomes and the trans-Golgi network, the route delivering MT1-MMP to invadopodia (Poincloux et al., 2009). Hence, in addition to dephosphorylation of PI(4,5)P₂, SYNJ2 likely processes PI(3,5)P₂ to fine tune the PI(3)P pool of early endosomes and coordinate both exocytosis of MT1-MMP and recycling of integrins, as well as EGFR.

When introduced into animals, shSYNJ2 MDA-MB-231 cells severely lost metastatic potential, due to reduced ability to reach lymph nodes and blood vessels (FIGS. 13A-H). In an attempt to integrate these results and the in vitro phenotypes, outlined in FIG. 15 are mechanisms underlying the roles of SYNJ2 in cellular motility. Accordingly, a key event entails EGF-induced up-regulation of SYNJ2, and consequent depletion of three phosphoinositides: PI(4,5)P₂, PI(3,4,5)P₃ and PI(3,5)P₂. SYNJ2-mediated PI(4,5)P₂ dephosphorylation is paralleled by degradation of PI(4,5)P₂ by phospholipase C-gamma, and phosphorylation by PI3K, which generates PI(3,4,5)P₃. Collectively, stimulation of the three enzymes by EGF dissociates a group of PI(4,5)P₂ binders from the plasma membrane, and also generates PI(4,5)P₂-devoid endocytic vesicles. Concurrently, SYNJ2 converts PI(3,4,5)P₃ into PI(3,4)P₂, which is essential for invadopodia formation. In line with this model, it has been reported that PI3K is needed for invadopodia formation. Once in place, PI(3,4)P₂ binds TKS5 and nucleates a Dynamin and Cortactin-centered complex that enables Cofilin to generate actin barbed ends within invadopodia. According to the present results, SYNJ2 is involved also in the next invadopodia maturation steps, namely secretion of MMPs and delivery of MT1-MMP and other surface molecules, such as CD44. In a similar way, SYNJ2 controls delivery of EGFRs and integrin to the leading edge, and likely activates Cofillin, a pivotal event dictating formation of lamellipodial protrusions.

In line with SYNJ2's contribution to cell migration in vitro and metastasis in animals, the present survey of breast cancer specimens observed significant up-regulation of SYNJ2 mRNA and protein levels in aggressive subtypes of the disease. In addition, using data from two cohorts, an association between high SYNJ2 mRNA expression and shorter survival of breast cancer patients was observed.

In summary, the present study attributes essential metastasis-initiating events to EGF-induced local activation of PI3K and global up-regulation of SYNJ2, whose sequential action upon PI(4,5)P₂ regulates actin dynamics at the leading edge, as well as generates PI(3,4)P₂, the signpost of invadopodia. Furthermore, the present study identified various compounds that selectively inhibited generation of PI(3,4)P2 by SYNJ2.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### REFERENCES

### Other References are Included in the Text

Amit, I., Citri, A., Shay, T., Lu, Y., Katz, M., Zhang, F., Tarcic, G., Siwak, D., Lahad, J., Jacob-Hirsch, J., et al. (2007). A module of negative feedback regulators defines growth factor signaling. Nat Genet 39, 503-512.
Augoff, K., Das, M., Bialkowska, K., McCue, B., Plow, E. F., and Sossey-Alaoui, K. (2011). miR-31 is a broad regulator of beta1-integrin expression and function in cancer cells. Molecular cancer research: MCR 9, 1500-1508.
Bos, P. D., Zhang, X. H., Nadal, C., Shu, W., Gomis, R. R., Nguyen, D. X., Minn, A. J., van de Vijver, M. J., Gerald, W. L., Foekens, J. A., et al. (2009). Genes that mediate breast cancer metastasis to the brain. Nature 459, 1005-1009.
Catimel, B., Schieber, C., Condron, M., Patsiouras, H., Connolly, L., Catimel, J., Nice, E. C., Burgess, A. W., and Holmes, A. B. (2008). The PI(3,5)P2 and PI(4,5)P2 interactomes. J Proteome Res 7, 5295-5313.
Chuang, Y. Y., Tran, N. L., Rusk, N., Nakada, M., Berens, M. E., and Symons, M. (2004). Role of synaptojanin 2 in glioma cell migration and invasion. Cancer research 64, 8271-8275.
Courtneidge, S. A., Azucena, E. F., Pass, I., Seals, D. F., and Tesfay, L. (2005). The SRC substrate TksS, podosomes (invadopodia), and cancer cell invasion. Cold Spring Harb Symp Quant Biol 70, 167-171.
Cremona, 0., Di Paolo, G., Wenk, M. R., Luthi, A., Kim, W. T., Takei, K., Daniell, L., Nemoto, Y., Shears, S. B., Flavell, R. A., et al. (1999). Essential role of phosphoinositide metabolism in synaptic vesicle recycling. Cell 99, 179-188.
Ehrlich, M., Boll, W., Van Oijen, A., Hariharan, R., Chandran, K., Nibert, M. L., and Kirchhausen, T. (2004). Endocytosis by random initiation and stabilization of clathrin-coated pits. Cell 118, 591-605.
Gewinner, C., Wang, Z. C., Richardson, A., Teruya-Feldstein, J., Etemadmoghadam, D., Bowtell, D., Barretina, J., Lin, W. M., Rameh, L., Salmena, L., et al. (2009). Evidence that inositol polyphosphate 4-phosphatase type II is a tumor suppressor that inhibits PI3K signaling. Cancer cell 16, 115-125.
Goh, L. K., Huang, F., Kim, W., Gygi, S., and Sorkin, A. (2010). Multiple mechanisms collectively regulate clathrin-mediated endocytosis of the epidermal growth factor receptor. The Journal of cell biology 189, 871-883.
Gruenberg, J., and Stenmark, H. (2004). The biogenesis of multivesicular endosomes. Nat Rev Mol Cell Biol 5, 317-323.
Guo, W., and Giancotti, F. G. (2004). Integrin signalling during tumour progression. Nat Rev Mol Cell Biol 5, 816-826.
Howes, M. T., Kirkham, M., Riches, J., Cortese, K., Walser, P. J., Simpson, F., Hill, M. M., Jones, A., Lundmark, R., Lindsay, M. R., et al. (2010). Clathrin-independent carriers form a high capacity endocytic sorting system at the leading edge of migrating cells. The Journal of cell biology 190, 675-691.
Jefferson, A. B., and Majerus, P. W. (1996). Mutation of the conserved domains of two inositol polyphosphate 5-phosphatases. Biochemistry 35, 7890-7894.
Kaksonen, M., Sun, Y., and Drubin, D. G. (2003). A pathway for association of receptors, adaptors, and actin during endocytic internalization. Cell 115, 475-487.
Kruchten, A. E., and McNiven, M. A. (2006). Dynamin as a mover and pincher during cell migration and invasion. Journal of cell science 119, 1683-1690.
Macia, E., Ehrlich, M., Massol, R., Boucrot, E., Brunner, C., and Kirchhausen, T. (2006). Dynasore, a cell-permeable inhibitor of dynamin. Dev Cell 10, 839-850.
Malecz, N., McCabe, P. C., Spaargaren, C., Qiu, R., Chuang, Y., and Symons, M. (2000). Synaptojanin 2, a novel Rac1 effector that regulates clathrin-mediated endocytosis. Curr Biol 10, 1383-1386.
Mani, M., Lee, S. Y., Lucast, L., Cremona, 0., Di Paolo, G., De Camilli, P., and Ryan, T. A. (2007). The dual phosphatase activity of synaptojaninl is required for both efficient synaptic vesicle endocytosis and reavailability at nerve terminals. Neuron 56, 1004-1018.
Minn, A. J., Gupta, G. P., Siegel, P. M., Bos, P. D., Shu, W., Giri, D. D., Viale, A., Olshen, A. B., Gerald, W. L., and Massague, J. (2005). Genes that mediate breast cancer metastasis to lung. Nature 436, 518-524.
Mosesson, Y., Mills, G. B., and Yarden, Y. (2008). Derailed endocytosis: an emerging feature of cancer. Nat Rev Cancer 8, 835-850.
Mouneimne, G., DesMarais, V., Sidani, M., Scemes, E., Wang, W., Song, X., Eddy, R., and Condeelis, J. (2006). Spatial and temporal control of cofilin activity is required for directional sensing during chemotaxis. Curr Biol 16, 2193-2205.
Muller, P. A. J., Caswell, P. T., Doyle, B., Iwanicki, M. P., Tan, E. H., Karim, S., Lukashchuk, N., Gillespie, D. A., Ludwig, R. L., Gosseline, P., et al. (2010). Mutant p53 drives invasion by promoting integrin recycling. Cell 139, 1327-1341.
Murphy, D. A., and Courtneidge, S. A. (2011). The ‘ins’ and ‘outs’ of podosomes and invadopodia: characteristics, formation and function. Nat Rev Mol Cell Biol 12, 413-426.
Oikawa, T., Itoh, T., and Takenawa, T. (2008). Sequential signals toward podosome formation in NIH-src cells. The Journal of cell biology 182, 157-169.
Oser, M., Yamaguchi, H., Mader, C. C., Bravo-Cordero, J. J., Arias, M., Chen, X., Desmarais, V., van Rheenen, J., Koleske, A. J., and Condeelis, J. (2009). Cortactin regulates cofilin and N-WASp activities to control the stages of invadopodium assembly and maturation. The Journal of cell biology 186, 571-587.
Palamidessi, A., Frittoli, E., Garre, M., Faretta, M., Mione, M., Testa, I., Diaspro, A., Lanzetti, L., Scita, G., and Di Fiore, P. P. (2008). Endocytic trafficking of Rac is required for the spatial restriction of signaling in cell migration. Cell 134, 135-147.
Poincloux, R., Lizarraga, F., and Chavrier, P. (2009). Matrix invasion by tumour cells: a focus on MT1-MMP trafficking to invadopodia. Journal of cell science 122, 3015-3024.
Ridley, A. J. (2011). Life at the leading edge. Cell 145, 1012-1022.
Saarikangas, J., Zhao, H., and Lappalainen, P. (2010). Regulation of the actin cytoskeleton-plasma membrane interplay by phosphoinositides. Physiol Rev 90, 259-289.
Sossey-Alaoui, K., Downs-Kelly, E., Das, M., Izem, L., Tubbs, R., and Plow, E. F. (2011). WAVE3, an actin remodeling protein, is regulated by the metastasis suppressor microRNA, miR-31, during the invasion-metastasis cascade. International journal of cancer Journal international du cancer 129, 1331-1343.
Valastyan, S., Chang, A., Benaich, N., Reinhardt, F., and Weinberg, R. A. (2011). Activation of miR-31 function in already-established metastases elicits metastatic regression. Genes & development 25, 646-659.
Valastyan, S., Reinhardt, F., Benaich, N., Calogrias, D., Szasz, A. M., Wang, Z. C., Brock, J. E., Richardson, A. L., and Weinberg, R. A. (2009). A pleiotropically acting microRNA, miR-31, inhibits breast cancer metastasis. Cell 137, 1032-1046.
van Rheenen, J., Song, X., van Roosmalen, W., Cammer, M., Chen, X., Desmarais, V.,
Yip, S. C., Backer, J. M., Eddy, R. J., and Condeelis, J. S. (2007). EGF-induced PIP2 hydrolysis releases and activates cofilin locally in carcinoma cells. The Journal of cell biology 179, 1247-1259.
Wang, Y., and McNiven, M. A. (2012). Invasive matrix degradation at focal adhesions occurs via protease recruitment by a FAK-p130Cas complex. The Journal of cell biology.
Yamaguchi, H., Yoshida, S., Muroi, E., Yoshida, N., Kawamura, M., Kouchi, Z., Nakamura, Y., Sakai, R., and Fukami, K. (2011). Phosphoinositide 3-kinase signaling pathway mediated by p110alpha regulates invadopodia formation. The Journal of cell biology 193, 1275-1288.
Yu, W. H., Woessner, J. F., Jr., McNeish, J. D., and Stamenkovic, I. (2002). CD44 anchors the assembly of matrilysin/MMP-7 with heparin-binding epidermal growth factor precursor and ErbB4 and regulates female reproductive organ remodeling. Genes & development 16, 307-323.
Yuan, T. L., and Cantley, L. C. (2008). PI3K pathway alterations in cancer: variations on a theme. Oncogene 27, 5497-5510.

1. A method of preventing tumor metastasis with the proviso that said tumor is not glioma, the method comprising administering to a subject in need thereof a therapeutically effective amount of an inhibitor of synaptojanin 2 (SYNJ2), thereby preventing tumor metastasis.

2. (canceled)

3. A method of treating cancer the method comprising, administering to a subject in need thereof a therapeutically effective amount of an inhibitor of synaptojanin 2 (SYNJ2) and an inhibitor of a cell surface receptor associated with an onset or progression of cancer, thereby treating cancer.

4. (canceled)

5. The method of claim 3, wherein said cell surface receptor associated with said onset or progression of cancer is a receptor tyrosine kinase.

6. The method of claim 5, wherein said receptor tyrosine kinase is an ErbB receptor.

7. The method of claim 6, wherein said ErbB receptor is Epidermal Growth Factor Receptor (EGFR).

8. A method of identifying a putative inhibitor of tumor metastasis, the method comprising analyzing SYNJ2-mediated processing of PI(3,4,5)P₃ to PI(3,4)P₂ in the presence of a test agent, wherein a decreased processing of PI(3,4,5)P₃ to PI(3,4)P₂ in said presence of said test agent as compared to same in an absence thereof is indicative of a putative inhibitor of tumor metastasis.

9. The method of claim 8, wherein said analyzing SYNJ2-mediated processing of PI(3,4,5)P₃ to PI(3,4)P₂ is performed by a competition assay.

10. The method of claim 9, wherein said competition assay assays displacement of a PI(3,4)P₂ binding domain from a complex comprising said PI(3,4)P₂ binding domain bound to PI(3,4)P₂.

11. The method of claim 9, wherein said competition assay is a fluorescence polarization competitive assay.

12. A method of prognosing cancer in a subject in need thereof, the method comprising determining a level or activity of SYNJ2 in a cancer cell of the subject, wherein an upregulation in said level of activity of said SYNJ2 in said cancer cell of the subject compared to same in a cell of an unaffected control sample, is indicative of a poor prognosis.

13. The method of claim 12, further comprising augmenting said prognosis using a Gold standard method.

14. The method of claim 13, wherein said Gold standard method comprises detection of a marker.

15. The method of claim 14, wherein said marker is selected from the group consisting of HER-2 and estrogen receptor (ER).

16. The method of claim 1, wherein said metastasis is EGF dependent.

17. The method of claim 3, wherein said cancer is breast cancer.

18. The method of claim 1, wherein said inhibitor of SYNJ2 is selected from the group consisting of a small molecule, an antibody, a peptide and a nucleic acid silencing agent.

19. The method of claim 18, wherein said small molecule is selected from the molecules listed in Table 2.

20. An article of manufacture for the treatment of cancer or prevention of cancer metastasis, comprising a packaging material packaging an inhibitor of SYNJ2 and an inhibitor of a cell surface receptor associated with an onset or progression of cancer.

21. The method of claim 3, wherein said inhibitor of said cell surface receptor associated with said onset or progression of cancer is an antibody.

22. The method of claim 3, wherein said inhibitor of said cell surface receptor associated with said onset or progression of cancer is a small molecule inhibitor.

23. The method of claim 12, wherein said cancer is breast cancer.

24. The method of claim 3, wherein said inhibitor of SYNJ2 is selected from the group consisting of a small molecule, an antibody, a peptide and a nucleic acid silencing agent.

25. The method of claim 24, wherein said small molecule is selected from the molecules listed in Table 2.

26. The article of manufacture of claim 20, wherein said inhibitor of said cell surface receptor associated with said onset or progression of cancer is an antibody.

27. The article of manufacture of claim 20, wherein said inhibitor of said cell surface receptor associated with said onset or progression of cancer is a small molecule inhibitor.

28. The method of claim 10, wherein said competition assay is a fluorescence polarization competitive assay.


SEQUENCE LISTING (16 sequences)

SEQ ID NO: 1 - DNA, 8982 bp, Homo sapiens
atggccctgagcaaagggctgcggctgctggggcgcctgggggccgagggggactgtagcgtgctgctggaggcgcgcggccgcgacgactgcctgctgttcgaggccggcacggtggccacgctggctccagaagaaaaggaagtcattaaaggacagtatggcaagctcacggacgcgtacggctgcctgggggagctgaggctgaaatctggtggcacgtctctgagcttcctggtgttggtgacaggctgcacatctgtgggcagaattccagatgctgaaatctacaaaatcactgccactgacttttaccctcttcaggaagaggccaaggaggaggaacgcctcatagctttgaagaaaatcctcagctcgggggtgttctatttctcatggccaaacgatgggtctcgctttgacctgactgtccgcacgcagaagcagggggatgacagctctgaatgggggaactccttcttctggaaccagctgttgcacgtgcccttgaggcagcaccaggtgagctgctgtgactggctgctgaagatcatctgcggggtggtcaccatccgcaccgtgtatgcctcccacaagcaggccaaggcctgcctcgtctctcgcgttagctgtgagcgcacaggcactcgcttccacacccgtggcgtgaacgacgacggccatgtgtccaacttcgtggagacagagcagatgatttacatggacgatggagtgtcatcttttgtccagatcagaggctccgttccgctgttctgggaacagccagggcttcaggttggctcccatcatctgagactccacagaggcctggaagccaatgcccctgctttcgacaggcacatggtgcttctgaaggagcagtacgggcagcaggtggtcgtgaaccttctgggaagcagaggcggagaggaggtgctcaacagagccttcaagaagctgctctgggcttcttgccacgcgggcgacacgcctatgatcaattttgacttccatcagtttgccaaaggtgggaagctagagaaattggagaccctcttgaggccacagttaaagctgcactgggaagacttcgatgtgttcacaaagggggagaacgtcagtccacgttttcagaaaggcactttgcggatgaactgtcttgactgcctggaccgaaccaacactgtgcagagcttcatcgcgctcgaggtcctgcatctgcagctcaagaccctggggctgagttcaaaacccatcgttgaccgctttgtggagtccttcaaagccatgtggtctctgaatggccacagcctgagcaaggtgttcacaggcagcagagccctggaagggaaggccaaggtggggaagctgaaggatggagcccggtccatgtctcgaaccatccagtccaacttcttcgacggggtgaagcaggaggccatcaagctgctgctggttggggacgtctacggcgaggaggtggcagacaaagggggcatgctgctggacagcacggcgctcctggtgactcccaggatcctgaaagctatgactgagcgtcagtccgaattcacaaatttcaagcggatccggattgctatggggacctggaacgtgaacggaggaaagcagttccggagcaacgtgctcaggacggcggagctgacagactggctgctcgactcgccccagctctcgggagctaccgactcccaggatgacagcagcccagctgacatatttgctgtggggtttgaagagatggtggaattgagcgcagggaatattgtcaatgccagtactaccaacaagaagatgtggggtgaacagcttcagaaagccatctcacgctctcatagatacattctgttgacttcggcacagctggtgggcgtctgtctttatatctttgtacgtccataccatgtcccgttcatcagggacgtagccatcgacacagtgaagacgggcatggggggcaaggcggggaacaagggcgccgtcggcatccgcttccagttccacagcaccagcttctgcttcatatgtagtcacctgacggccgggcagtcccaggtgaaggagcggaatgaagactacaaggagatcacccagaaactctgcttcccaatggggagaaatgttttttctcatgattatgtattttggtgtggcgatttcaactaccgcattgatcttacttatgaagaagtcttctattttgttaaacgccaagactggaagaaacttctggaatttgatcaactacagctacagaaatcaagtggaaaaatttttaaggactttcacgaaggagccattaactttggacccacctacaagtatgacgttggctcagccgcctacgatacaagcgacaaatgccgcacccccgcctggacagacagggtgctgtggtggaggaagaaacatccctttgataaaacagctggagaactcaaccttctagacagtgatctagatgttgacaccaaagtcagacacacctggtctcctggtgccctgcagtattatggtcgtgcggagctacaagcgtctgatcacagacctgtgctggcgatcgtggaggtggaagttcaggaagtcgatgtgggtgctcgggagagggttttccaggaagtgtcctccttccagggccccctggatgccactgttgtagtaaaccttcaatcaccgaccttagaagagaaaaacgagtttccagaggacctgcgtactgagctcatgcagaccttggggagttatgggacaattgttcttgtcaggatcaaccaagggcagatgctggtaacttttgcagacagtcactcggctctcagtgtcctggacgtggacggtatgaaggtgaaaggcagagcagtgaagattagaccgaagaccaaggactggctgaaaggtttgcgagaggagatcattcggaaacgagacagcatggcccccgtgtctcccactgccaactcctgtttgctggaggaaaactttgacttcacaagtttggactatgagtcagaaggggatattcttgaagacgatgaagactacttggtggatgaattcaatcagcctggagtctcggacagtgaactcgggggagacgacctctctgatgtccccggccccacagcactggctcctcccagcaagtcacctgctctcaccaaaaagaagcagcatccaacgtacaaagatgacgcggacctggtggagctcaagcgggagctggaagccgtcggggagttccgccaccgttctccgagcaggtctctgtcggtccccaaccggcctcggccacctcaacccccgcagagacccccccctccaaccggtttaatggtgaaaaagtcggcttcagatgcgtccatctcctccggcacccatggacagtattcaattttgcagacggcaagacttctaccaggagcacctcagcaacctcccaaggctcggactggaataagtaaaccttataatgtcaagcagatcaaaaccaccaatgcccaggaggcagaagcagcaatccggtgtctcctggaagccagaggaggtgcctccgaagaagccctaagtgccgtggccccaagggaccttgaagcatcctctgaaccagagcccacaccgggggcagccaaaccagagaccccacaggcgcccccactccttccccgtcggcccccacccagagttcctgccatcaagaagccaaccttgagaaggacaggaaagcccctgtcaccggaagaacagtttgagcaacagactgtccattttacaatcgggcccccggagacaagcgttgaggcccctcctgtcgtgacagcccctcgagtccctcctgttcccaaaccaagaacatttcagcctgggaaagctgcagagaggccaagccacaggaagccagcatcagacgaagcccctcctggggcaggagcctctgtgccaccacctctggaggcgccgcctcttgtgcccaaggtacccccgaggaggaagaagtcagcccccgcagccttccacctgcaggtcctgcagagcaacagccagcttctccagggcctcacttacaatagcagtgacagcccctctgggcacccacctgccgcgggcaccgtcttcccacaaggggactttctcagcacttcatctgctacaagccccgacagcgatggcaccaaagcgatgaagccagaggcagccccacttcttggtgattatcaggaccccttctggaaccttcttcaccaccctaaactgttgaataacacttggctttctaagagctcagaccctttggactcaggaaccaggagccccaaaagagatcccatagacccagtgtcagctggcgcttcagctgccaaggcagagctgccaccagatcatgaacacaaaaccttaggtcactgggtgacaatcagtgaccaagaaaagaggacagcactgcaggtgtttgacccactggcaaaaacatgaatggccctgagcaaagggctgcggctgctggggcgcctgggggccgagggggactgtagcgtgctgctggaggcgcgcggccgcgacgactgcctgctgttcgaggccggcacggtggccacgctggctccagaagaaaaggaagtcattaaaggacagtatggcaagctcacggacgcgtacggctgcctgggggagctgaggctgaaatctggtggcacgtctctgagcttcctggtgttggtgacaggctgcacatctgtgggcagaattccagatgctgaaatctacaaaatcactgccactgacttttaccctcttcaggaagaggccaaggaggaggaacgcctcatagctttgaagaaaatcctcagctcgggggtgttctatttctcatggccaaacgatgggtctcgctttgacctgactgtccgcacgcagaagcagggggatgacagctctgaatgggggaactccttcttctggaaccagctgttgcacgtgcccttgaggcagcaccaggtgagctgctgtgactggctgctgaagatcatctgcggggtggtcaccatccgcaccgtgtatgcctcccacaagcaggccaaggcctgcctcgtctctcgcgttagctgtgagcgcacaggcactcgcttccacacccgtggcgtgaacgacgacggccatgtgtccaacttcgtggagacagagcagatgatttacatggacgatggagtgtcatcttttgtccagatcagaggctccgttccgctgttctgggaacagccagggcttcaggttggctcccatcatctgagactccacagaggcctggaagccaatgcccctgctttcgacaggcacatggtgcttctgaaggagcagtacgggcagcaggtggtcgtgaaccttctgggaagcagaggcggagaggaggtgctcaacagagccttcaagaagctgctctgggcttcttgccacgcgggcgacacgcctatgatcaattttgacttccatcagtttgccaaaggtgggaagctagagaaattggagaccctcttgaggccacagttaaagctgcactgggaagacttcgatgtgttcacaaagggggagaacgtcagtccacgttttcagaaaggcactttgcggatgaactgtcttgactgcctggaccgaaccaacactgtgcagagcttcatcgcgctcgaggtcctgcatctgcagctcaagaccctggggctgagttcaaaacccatcgttgaccgctttgtggagtccttcaaagccatgtggtctctgaatggccacagcctgagcaaggtgttcacaggcagcagagccctggaagggaaggccaaggtggggaagctgaaggatggagcccggtccatgtctcgaaccatccagtccaacttcttcgacggggtgaagcaggaggccatcaagctgctgctggttggggacgtctacggcgaggaggtggcagacaaagggggcatgctgctggacagcacggcgctcctggtgactcccaggatcctgaaagctatgactgagcgtcagtccgaattcacaaatttcaagcggatccggattgctatggggacctggaacgtgaacggaggaaagcagttccggagcaacgtgctcaggacggcggagctgacagactggctgctcgactcgccccagctctcgggagctaccgactcccaggatgacagcagcccagctgacatatttgctgtggggtttgaagagatggtggaattgagcgcagggaatattgtcaatgccagtactaccaacaagaagatgtggggtgaacagcttcagaaagccatctcacgctctcatagatacattctgttgacttcggcacagctggtgggcgtctgtctttatatctttgtacgtccataccatgtcccgttcatcagggacgtagccatcgacacagtgaagacgggcatggggggcaaggcggggaacaagggcgccgtcggcatccgcttccagttccacagcaccagcttctgcttcatatgtagtcacctgacggccgggcagtcccaggtgaaggagcggaatgaagactacaaggagatcacccagaaactctgcttcccaatggggagaaatgttttttctcatgattatgtattttggtgtggcgatttcaactaccgcattgatcttacttatgaagaagtcttctattttgttaaacgccaagactggaagaaacttctggaatttgatcaactacagctacagaaatcaagtggaaaaatttttaaggactttcacgaaggagccattaactttggacccacctacaagtatgacgttggctcagccgcctacgatacaagcgacaaatgccgcacccccgcctggacagacagggtgctgtggtggaggaagaaacatccctttgataaaacagctggagaactcaaccttctagacagtgatctagatgttgacaccaaagtcagacacacctggtctcctggtgccctgcagtattatggtcgtgcggagctacaagcgtctgatcacagacctgtgctggcgatcgtggaggtggaagttcaggaagtcgatgtgggtgctcgggagagggttttccaggaagtgtcctccttccagggccccctggatgccactgttgtagtaaaccttcaatcaccgaccttagaagagaaaaacgagtttccagaggacctgcgtactgagctcatgcagaccttggggagttatgggacaattgttcttgtcaggatcaaccaagggcagatgctggtaacttttgcagacagtcactcggctctcagtgtcctggacgtggacggtatgaaggtgaaaggcagagcagtgaagattagaccgaagaccaaggactggctgaaaggtttgcgagaggagatcattcggaaacgagacagcatggcccccgtgtctcccactgccaactcctgtttgctggaggaaaactttgacttcacaagtttggactatgagtcagaaggggatattcttgaagacgatgaagactacttggtggatgaattcaatcagcctggagtctcggacagtgaactcgggggagacgacctctctgatgtccccggccccacagcactggctcctcccagcaagtcacctgctctcaccaaaaagaagcagcatccaacgtacaaagatgacgcggacctggtggagctcaagcgggagctggaagccgtcggggagttccgccaccgttctccgagcaggtctctgtcggtccccaaccggcctcggccacctcaacccccgcagagacccccccctccaaccggtttaatggtgaaaaagtcggcttcagatgcgtccatctcctccggcacccatggacagtattcaattttgcagacggcaagacttctaccaggagcacctcagcaacctcccaaggctcggactggaataagtaaaccttataatgtcaagcagatcaaaaccaccaatgcccaggaggcagaagcagcaatccggtgtctcctggaagccagaggaggtgcctccgaagaagccctaagtgccgtggccccaagggaccttgaagcatcctctgaaccagagcccacaccgggggcagccaaaccagagaccccacaggcgcccccactccttccccgtcggcccccacccagagttcctgccatcaagaagccaaccttgagaaggacaggaaagcccctgtcaccggaagaacagtttgagcaacagactgtccattttacaatcgggcccccggagacaagcgttgaggcccctcctgtcgtgacagcccctcgagtccctcctgttcccaaaccaagaacatttcagcctgggaaagctgcagagaggccaagccacaggaagccagcatcagacgaagcccctcctggggcaggagcctctgtgccaccacctctggaggcgccgcctcttgtgcccaaggtacccccgaggaggaagaagtcagcccccgcagccttccacctgcaggtcctgcagagcaacagccagcttctccagggcctcacttacaatagcagtgacagcccctctgggcacccacctgccgcgggcaccgtcttcccacaaggggactttctcagcacttcatctgctacaagccccgacagcgatggcaccaaagcgatgaagccagaggcagccccacttcttggtgattatcaggaccccttctggaaccttcttcaccaccctaaactgttgaataacacttggctttctaagagctcagaccctttggactcaggaaccaggagccccaaaagagatcccatagacccagtgtcagctggcgcttcagctgccaaggcagagctgccaccagatcatgaacacaaaaccttaggtcactgggtgacaatcagtgaccaagaaaagaggacagcactgcaggtgtttgacccactggcaaaaacatga

SEQ ID NO: 2 - protein, 1496 aa, Homo sapiens
MALSKGLRLLGRLGAEGDCSVLLEARGRDDCLLFEAGTVATLAPEEKEVIKGQYGKLTDAYGCLGELRLKSGGTSLSFLVLVTGCTSVGRIPDAEIYKITATDFYPLQEEAKEEERLIALKKILSSGVFYFSWPNDGSRFDLTVRTQKQGDDSSEWGNSFFWNQLLHVPLRQHQVSCCDWLLKIICGVVTIRTVYASHKQAKACLVSRVSCERTGTRFHTRGVNDDGHVSNFVETEQMIYMDDGVSSFVQIRGSVPLFWEQPGLQVGSHHLRLHRGLEANAPAFDRHMVLLKEQYGQQVVVNLLGSRGGEEVLNRAFKKLLWASCHAGDTPMINFDFHQFAKGGKLEKLETLLRPQLKLHWEDFDVFTKGENVSPRFQKGTLRMNCLDCLDRTNTVQSFIALEVLHLQLKTLGLSSKPIVDRFVESFKAMWSLNGHSLSKVFTGSRALEGKAKVGKLKDGARSMSRTIQSNFFDGVKQEAIKLLLVGDVYGEEVADKGGMLLDSTALLVTPRILKAMTERQSEFTNFKRIRIAMGTWNVNGGKQFRSNVLRTAELTDWLLDSPQLSGATDSQDDSSPADIFAVGFEEMVELSAGNIVNASTTNKKMWGEQLQKAISRSHRYILLTSAQLVGVCLYIFVRPYHVPFIRDVAIDTVKTGMGGKAGNKGAVGIRFQFHSTSFCFICSHLTAGQSQVKERNEDYKEITQKLCFPMGRNVFSHDYVFWCGDFNYRIDLTYEEVFYFVKRQDWKKLLEFDQLQLQKSSGKIFKDFHEGAINFGPTYKYDVGSAAYDTSDKCRTPAWTDRVLWWRKKHPFDKTAGELNLLDSDLDVDTKVRHTWSPGALQYYGRAELQASDHRPVLAIVEVEVQEVDVGARERVFQEVSSFQGPLDATVVVNLQSPTLEEKNEFPEDLRTELMQTLGSYGTIVLVRINQGQMLVTFADSHSALSVLDVDGMKVKGRAVKIRPKTKDWLKGLREEIIRKRDSMAPVSPTANSCLLEENFDFTSLDYESEGDILEDDEDYLVDEFNQPGVSDSELGGDDLSDVPGPTALAPPSKSPALTKKKQHPTYKDDADLVELKRELEAVGEFRHRSPSRSLSVPNRPRPPQPPQRPPPPTGLMVKKSASDASISSGTHGQYSILQTARLLPGAPQQPPKARTGISKPYNVKQIKTTNAQEAEAAIRCLLEARGGASEEALSAVAPRDLEASSEPEPTPGAAKPETPQAPPLLPRRPPPRVPAIKKPTLRRTGKPLSPEEQFEQQTVHFTIGPPETSVEAPPVVTAPRVPPVPKPRTFQPGKAAERPSHRKPASDEAPPGAGASVPPPLEAPPLVPKVPPRRKKSAPAAFHLQVLQSNSQLLQGLTYNSSDSPSGHPPAAGTVFPQGDFLSTSSATSPDSDGTKAMKPEAAPLLGDYQDPFWNLLHHPKLLNNTWLSKSSDPLDSGTRSPKRDPIDPVSAGASAAKAELPPDHEHKTLGHWVTISDQEKRTALQVFDPLAKT

SEQ ID NO: 3 - DNA, 21 bp, Artificial sequence
  Examples of a small interfering RNA molecule sequence, targeting coding region 1612-1633 (SJ2-1)
aacgtgaacggaggaaagcag

SEQ ID NO: 4 - DNA, 21 bp, Artificial sequence
  Examples of a small interfering RNA molecule sequence, targeting the 3? untranslated region (SJ2-2)
ctcttgctgatacgcgatatt

SEQ ID NO: 5 - RNA, 19 nt, Artificial sequence
  Example of an siRNA sequence that successfully downregulates SYNJ2 mRNA levels
gaagaaacaucccuuugau

SEQ ID NO: 6 - RNA, 19 nt, Artificial sequence
  Example of an siRNA sequence that successfully downregulates SYNJ2 mRNA levels
ggacagcacugcagguguu

SEQ ID NO: 7 - DNA, 59 bp, Artificial sequence
  Example of an shRNA sequence that successfully downregulates SYNJ2 mRNA levels
ccggcctacgatacaagcgacaaatctcgaagatttgtcgcttgtatcgtaggtttttg

SEQ ID NO: 8 - DNA, 58 bp, Artificial sequence
  Example of an shRNA sequence that successfully downregulates SYNJ2 mRNA levels
ccggcgagaggagatcattcggaaactcgagtttccgaatgatctcctctcgtttttg

SEQ ID NO: 9 - DNA, 58 bp, Artificial sequence
  Example of an shRNA sequence that successfully downregulates SYNJ2 mRNA levels
ccggccggaagaacagtttgagcaactcgagttgctcaaactgttcttccggtttttg

SEQ ID NO: 10 - DNA, 20 bp, Artificial sequence
  Example of an antisense oligonucleotide targeting coding region 1494-1475
ccctttgtctgccacctcct

SEQ ID NO: 11 - DNA, 19 bp, Artificial sequence
  Example of an antisense oligonucleotide targeting the 3′ UTR sequence
acccatcttgctctctccc

SEQ ID NO: 12 - DNA, 20 bp, Artificial sequence
  Example of an antisense oligonucleotide targeting coding region 2428-2409
tcttcctccaccacagcacc

SEQ ID NO: 13 - DNA, 738 bp, Artificial Sequence
  Recombinant TAPP1 PH domain derived from Homo sapiens
atggattacaaggacgacgacgataagggcggatccccttactttactcctaaaccacctcaagatagtacggttatcaaagctggatattgtgtaaaacaaggagcagtgatgaaaaactggaagagaagatattttcaattggatgaaaacacaataggctacttcaaatctgaactggaaaaggaacctcttcgcgtaataccacttaaagaggttcataaagtccaggaatgtaagcaaagcgacataatgatgagggacaacctctttgaaattgtaacaacgtctcgaactttctatgtgcaggctgatagccctgaagagatgcacagttggattaaagcagtctctggcgccattgtagcacagcggggtcccggcagatctgcgtcttctgagcatccccccggtccttcagaatccaaacacgctttccgtcctaccaacgcagccaccgccacctcacattccacagcctctcgcagcaactctttggtctcaacctttaccatggagaagcgaggattttacgagtctcttgccaaggtcaagccagggaacttcaaggtccagactgtctctccaagagaaccagcttccaaagtgactgaacaagctctgttaagacctcaaagtaaaaatggccctcaggaaaaagattgtgacctagtagacttggacgatgcgagccttccggtcagtgacgtgctcgagcaccaccaccaccaccactga

SEQ ID NO: 14 - protein, 245 aa, Artificial Sequence
  Recombinant TAPP1 PH domain derived from Homo sapiens
MDYKDDDDKGGSPYFTPKPPQDSTVIKAGYCVKQGAVMKNWKRRYFQLDENTIGYFKSELEKEPLRVIPLKEVHKVQECKQSDIMMRDNLFEIVTTSRTFYVQADSPEEMHSWIKAVSGAIVAQRGPGRSASSEHPPGPSESKHAFRPTNAATATSHSTASRSNSLVSTFTMEKRGFYESLAKVKPGNFKVQTVSPREPASKVTEQALLRPQSKNGPQEKDCDLVDLDDASLPVSDVLEHHHHHH

SEQ ID NO: 15 - protein, 225 aa, Homo sapiens
PYFTPKPPQDSTVIKAGYCVKQGAVMKNWKRRYFQLDENTIGYFKSELEKEPLRVIPLKEVHKVQECKQSDIMMRDNLFEIVTTSRTFYVQADSPEEMHSWIKAVSGAIVAQRGPGRSASSEHPPGPSESKHAFRPTNAATATSHSTASRSNSLVSTFTMEKRGFYESLAKVKPGNFKVQTVSPREPASKVTEQALLRPQSKNGPQEKDCDLVDLDDASLPVSDV

SEQ ID NO: 16 - DNA, 675 bp, Homo sapiens
ccttactttactcctaaaccacctcaagatagtacggttatcaaagctggatattgtgtaaaacaaggagcagtgatgaaaaactggaagagaagatattttcaattggatgaaaacacaataggctacttcaaatctgaactggaaaaggaacctcttcgcgtaataccacttaaagaggttcataaagtccaggaatgtaagcaaagcgacataatgatgagggacaacctctttgaaattgtaacaacgtctcgaactttctatgtgcaggctgatagccctgaagagatgcacagttggattaaagcagtctctggcgccattgtagcacagcggggtcccggcagatctgcgtcttctgagcatccccccggtccttcagaatccaaacacgctttccgtcctaccaacgcagccaccgccacctcacattccacagcctctcgcagcaactctttggtctcaacctttaccatggagaagcgaggattttacgagtctcttgccaaggtcaagccagggaacttcaaggtccagactgtctctccaagagaaccagcttccaaagtgactgaacaagctctgttaagacctcaaagtaaaaatggccctcaggaaaaagattgtgacctagtagacttggacgatgcgagccttccggtcagtgacgtg